SEQUENCE LISTING (105 sequences)

SEQ ID NO: 1 - DNA, 33 bp, Artificial Sequence
  Oligonucleotide
tgcacgtcatctacagtaggtcggtgcgtaggt

SEQ ID NO: 2 - DNA, 34 bp, Artificial Sequence
  Oligonucleotide
ctcactggagttcaagggacgatgagtggcgatg

SEQ ID NO: 3 - DNA, 34 bp, Artificial Sequence
  Oligonucleotide
catcgccactcatcgtcccttgaactccagtgag

SEQ ID NO: 4 - DNA, 18 bp, Artificial Sequence
  Oligonucleotide
ctgtggccagaagcgggg

SEQ ID NO: 5 - DNA, 25 bp, Artificial Sequence
  Oligonucleotide
tgttccttttgatcgtggtgattta

SEQ ID NO: 6 - DNA, 21 bp, Artificial Sequence
  Oligonucleotide
aacaaaactgctcgccagaac

SEQ ID NO: 7 - DNA, 20 bp, Artificial Sequence
  Oligonucleotide
catccccgttccagtgagtt

SEQ ID NO: 8 - DNA, 23 bp, Artificial Sequence
  Oligonucleotide
tttaactgttgaggtttagggct

SEQ ID NO: 9 - DNA, 25 bp, Artificial Sequence
  Oligonucleotide
aggggttttagttaaatgtcctttg

SEQ ID NO: 10 - DNA, 23 bp, Artificial Sequence
  Oligonucleotide
ccaaagctaagacccccgaaacc

SEQ ID NO: 11 - DNA, 19 bp, Artificial Sequence
  Oligonucleotide
cgtacacaccgcccgtcac

SEQ ID NO: 12 - DNA, 28 bp, Artificial Sequence
  Oligonucleotide
ttgcaaagttatttctagttaattcatt

SEQ ID NO: 13 - DNA, 22 bp, Artificial Sequence
  Oligonucleotide
ggtgatagattggtccaattgg

SEQ ID NO: 14 - DNA, 21 bp, Artificial Sequence
  Oligonucleotide
tgtatgaatggctccacgagg

SEQ ID NO: 15 - DNA, 20 bp, Artificial Sequence
  Oligonucleotide
aagctcaacacccactacct

SEQ ID NO: 16 - DNA, 18 bp, Artificial Sequence
  Oligonucleotide
tgtcactgggcaggcggt

SEQ ID NO: 17 - DNA, 25 bp, Artificial Sequence
  Oligonucleotide
aaaccctattgttgatatggactct

SEQ ID NO: 18 - DNA, 23 bp, Artificial Sequence
  Oligonucleotide
atggcattcctaatgcttaccga

SEQ ID NO: 19 - DNA, 23 bp, Artificial Sequence
  Oligonucleotide
ggataacagcgcaatcctattct

SEQ ID NO: 20 - DNA, 19 bp, Artificial Sequence
  Oligonucleotide
aggagtaggaggttggcca

SEQ ID NO: 21 - DNA, 22 bp, Artificial Sequence
  Oligonucleotide
aatgatggctagggtgacttca

SEQ ID NO: 22 - DNA, 24 bp, Artificial Sequence
  Oligonucleotide
acctccctgttcttatgaattcga

SEQ ID NO: 23 - DNA, 18 bp, Artificial Sequence
  Oligonucleotide
gccctgatcggcgcactg

SEQ ID NO: 24 - DNA, 25 bp, Artificial Sequence
  Oligonucleotide
atatgttgttcctaggaagattgta

SEQ ID NO: 25 - DNA, 25 bp, Artificial Sequence
  Oligonucleotide
attagtacgggaagggtataaccaa

SEQ ID NO: 26 - DNA, 28 bp, Artificial Sequence
  Oligonucleotide
gctatagcaataaaactaggaatagccc

SEQ ID NO: 27 - DNA, 22 bp, Artificial Sequence
  Oligonucleotide
cccatccctgagaatccaaaat

SEQ ID NO: 28 - DNA, 20 bp, Artificial Sequence
  Oligonucleotide
tggttcattgtccggagagt

SEQ ID NO: 29 - DNA, 20 bp, Artificial Sequence
  Oligonucleotide
ggtcgtggtgctggagttta

SEQ ID NO: 30 - DNA, 24 bp, Artificial Sequence
  Oligonucleotide
ctcctacctatctccccttttata

SEQ ID NO: 31 - DNA, 25 bp, Artificial Sequence
  Oligonucleotide
ctaactactaccgcattcctactac

SEQ ID NO: 32 - DNA, 20 bp, Artificial Sequence
  Oligonucleotide
agcgtggtaagggcgatgag

SEQ ID NO: 33 - DNA, 22 bp, Artificial Sequence
  Oligonucleotide
ggtgaggtaaaatggctgagtg

SEQ ID NO: 34 - DNA, 20 bp, Artificial Sequence
  Oligonucleotide
gcaggaacaggttgaacagt

SEQ ID NO: 35 - DNA, 24 bp, Artificial Sequence
  Oligonucleotide
cccctgtctttagatttacagtcc

SEQ ID NO: 36 - DNA, 21 bp, Artificial Sequence
  Oligonucleotide
caggagtaggagagagggagg

SEQ ID NO: 37 - DNA, 22 bp, Artificial Sequence
  Oligonucleotide
ccgaagcctggtaggataagaa

SEQ ID NO: 38 - DNA, 22 bp, Artificial Sequence
  Oligonucleotide
tttcttttcaccgtaggtggcc

SEQ ID NO: 39 - DNA, 25 bp, Artificial Sequence
  Oligonucleotide
tggcatcactatactactaacagac

SEQ ID NO: 40 - DNA, 20 bp, Artificial Sequence
  Oligonucleotide
aatcctagggctcagagcac

SEQ ID NO: 41 - DNA, 24 bp, Artificial Sequence
  Oligonucleotide
actattaggacttttcgcttcgaa

SEQ ID NO: 42 - DNA, 25 bp, Artificial Sequence
  Oligonucleotide
tttcatgatcacgccctcataatca

SEQ ID NO: 43 - DNA, 21 bp, Artificial Sequence
  Oligonucleotide
cccgatgcatacaccacatga

SEQ ID NO: 44 - DNA, 21 bp, Artificial Sequence
  Oligonucleotide
ggggaagtagcgtcttgtaga

SEQ ID NO: 45 - DNA, 20 bp, Artificial Sequence
  Oligonucleotide
gaatgggggcttcaatcggg

SEQ ID NO: 46 - DNA, 19 bp, Artificial Sequence
  Oligonucleotide
taccccctctagagcccac

SEQ ID NO: 47 - DNA, 21 bp, Artificial Sequence
  Oligonucleotide
attggccaccaatggtactga

SEQ ID NO: 48 - DNA, 22 bp, Artificial Sequence
  Oligonucleotide
tgctatagggtaaatacgggcc

SEQ ID NO: 49 - DNA, 21 bp, Artificial Sequence
  Oligonucleotide
tgttgggtggtgattagtcgg

SEQ ID NO: 50 - DNA, 28 bp, Artificial Sequence
  Oligonucleotide
cccatactagttattatcgaaaccatca

SEQ ID NO: 51 - DNA, 20 bp, Artificial Sequence
  Oligonucleotide
gcttcattcattgcccccac

SEQ ID NO: 52 - DNA, 20 bp, Artificial Sequence
  Oligonucleotide
gtgtaggtgtgccttgtggt

SEQ ID NO: 53 - DNA, 20 bp, Artificial Sequence
  Oligonucleotide
aggcctagtatgaggagcgt

SEQ ID NO: 54 - DNA, 25 bp, Artificial Sequence
  Oligonucleotide
ccgaaaccaaataattcaagcactg

SEQ ID NO: 55 - DNA, 20 bp, Artificial Sequence
  Oligonucleotide
agccctcctaatgacctccg

SEQ ID NO: 56 - DNA, 21 bp, Artificial Sequence
  Oligonucleotide
gctggagtggtaaaaggctca

SEQ ID NO: 57 - DNA, 21 bp, Artificial Sequence
  Oligonucleotide
acaaaatgccagtatcaggcg

SEQ ID NO: 58 - DNA, 22 bp, Artificial Sequence
  Oligonucleotide
cttttacccctaccatgagccc

SEQ ID NO: 59 - DNA, 22 bp, Artificial Sequence
  Oligonucleotide
tatctgcttcatccgccaacta

SEQ ID NO: 60 - DNA, 28 bp, Artificial Sequence
  Oligonucleotide
atagctactaagaagaattttatggaga

SEQ ID NO: 61 - DNA, 22 bp, Artificial Sequence
  Oligonucleotide
ggcacaatattggctaagaggg

SEQ ID NO: 62 - DNA, 20 bp, Artificial Sequence
  Oligonucleotide
cgccacttatccagtgaacc

SEQ ID NO: 63 - DNA, 23 bp, Artificial Sequence
  Oligonucleotide
tactctcataaccctcaacaccc

SEQ ID NO: 64 - DNA, 18 bp, Artificial Sequence
  Oligonucleotide
gttagggggtcggaggaa

SEQ ID NO: 65 - DNA, 25 bp, Artificial Sequence
  Oligonucleotide
aaaagctattgtgtaagctagtcat

SEQ ID NO: 66 - DNA, 25 bp, Artificial Sequence
  Oligonucleotide
cttacatcctcattactattctgcc

SEQ ID NO: 67 - DNA, 22 bp, Artificial Sequence
  Oligonucleotide
tctcactgcccaagaactatca

SEQ ID NO: 68 - DNA, 22 bp, Artificial Sequence
  Oligonucleotide
attatgagaatgactgcgccgg

SEQ ID NO: 69 - DNA, 19 bp, Artificial Sequence
  Oligonucleotide
cccggtaatgatgtcgggg

SEQ ID NO: 70 - DNA, 25 bp, Artificial Sequence
  Oligonucleotide
ctcttccccacaacaatattcatgt

SEQ ID NO: 71 - DNA, 21 bp, Artificial Sequence
  Oligonucleotide
acacctatcccccattctcct

SEQ ID NO: 72 - DNA, 25 bp, Artificial Sequence
  Oligonucleotide
ataataaaggtggatgcgacaatgg

SEQ ID NO: 73 - DNA, 22 bp, Artificial Sequence
  Oligonucleotide
taaggcgaggatgaaaccgata

SEQ ID NO: 74 - DNA, 23 bp, Artificial Sequence
  Oligonucleotide
ctccacttcaagtcaactaggac

SEQ ID NO: 75 - DNA, 20 bp, Artificial Sequence
  Oligonucleotide
atgccaacacagcagccatt

SEQ ID NO: 76 - DNA, 27 bp, Artificial Sequence
  Oligonucleotide
acgatttttttgatgtcattttgtgta

SEQ ID NO: 77 - DNA, 18 bp, Artificial Sequence
  Oligonucleotide
gtaagggtggggaagcga

SEQ ID NO: 78 - DNA, 23 bp, Artificial Sequence
  Oligonucleotide
actcctcctagacctaacctgac

SEQ ID NO: 79 - DNA, 20 bp, Artificial Sequence
  Oligonucleotide
aagcgcctatagcactcgaa

SEQ ID NO: 80 - DNA, 21 bp, Artificial Sequence
  Oligonucleotide
ttttggctcgtaagaaggcct

SEQ ID NO: 81 - DNA, 21 bp, Artificial Sequence
  Oligonucleotide
tagtggggttagcgatggagg

SEQ ID NO: 82 - DNA, 22 bp, Artificial Sequence
  Oligonucleotide
cccaatacgcaaaactaacccc

SEQ ID NO: 83 - DNA, 20 bp, Artificial Sequence
  Oligonucleotide
actctttcacccacagcacc

SEQ ID NO: 84 - DNA, 25 bp, Artificial Sequence
  Oligonucleotide
gtgttcttgtagttgaaatacaacg

SEQ ID NO: 85 - DNA, 20 bp, Artificial Sequence
  Oligonucleotide
tgatatttggcctcacggga

SEQ ID NO: 86 - DNA, 24 bp, Artificial Sequence
  Oligonucleotide
tataccctagccaaccccttaaac

SEQ ID NO: 87 - DNA, 20 bp, Artificial Sequence
  Oligonucleotide
gcattatcctcctgcttgca

SEQ ID NO: 88 - DNA, 20 bp, Artificial Sequence
  Oligonucleotide
tagggggttgtttgatcccg

SEQ ID NO: 89 - DNA, 21 bp, Artificial Sequence
  Oligonucleotide
gcttactggttgtcctccgat

SEQ ID NO: 90 - DNA, 25 bp, Artificial Sequence
  Oligonucleotide
tagtacataaaaacccaatccacat

SEQ ID NO: 91 - DNA, 21 bp, Artificial Sequence
  Oligonucleotide
gcagacctcctcattctaacc

SEQ ID NO: 92 - DNA, 27 bp, Artificial Sequence
  Oligonucleotide
tttatggtaccgtacaatattcatggt

SEQ ID NO: 93 - DNA, 23 bp, Artificial Sequence
  Oligonucleotide
tgtcttatttaaggggaacgtgt

SEQ ID NO: 94 - DNA, 21 bp, Artificial Sequence
  Oligonucleotide
cacatctctgccaaaccccaa

SEQ ID NO: 95 - DNA, 22 bp, Artificial Sequence
  Oligonucleotide
atctggttcctacttcagggtc

SEQ ID NO: 96 - DNA, 20 bp, Artificial Sequence
  Oligonucleotide
gttttgggccgtcagaggat

SEQ ID NO: 97 - DNA, 20 bp, Artificial Sequence
  Oligonucleotide
aaggaaaaccggacttcgca

SEQ ID NO: 98 - DNA, 22 bp, Artificial Sequence
  Oligonucleotide
tgtttgaagtcttggtttggtg

SEQ ID NO: 99 - DNA, 25 bp, Artificial Sequence
  Oligonucleotide
gaagataatgggattcagaactcag

SEQ ID NO: 100 - DNA, 20 bp, Artificial Sequence
  Oligonucleotide
gtgcagccttcatggtttcg

SEQ ID NO: 101 - DNA, 20 bp, Artificial Sequence
  Oligonucleotide
ccacagagagcatcgcaaga

SEQ ID NO: 102 - DNA, 21 bp, Artificial Sequence
  Oligonucleotide
ttgtctgtgatgtagccatca

SEQ ID NO: 103 - DNA, 21 bp, Artificial Sequence
  Oligonucleotide
tgtcaacaaccactttcacgg

SEQ ID NO: 104 - DNA, 21 bp, Artificial Sequence
  Oligonucleotide
gaatagcactcctccacaggg

SEQ ID NO: 105 - DNA, 24 bp, Artificial Sequence
  Oligonucleotid
tctacctgaattctggaacagccg

(19) United States
(12) Patent Application Publication
(10) Pub. No.: US 2022/0403470 A1
(43) Pub. Date: Dec. 22, 2022

(54) HUMAN MITOCHONDRIAL DNA SEQUENCING BY TARGETED AMPLIFICATION OF MULTIPLEX PROBES (MTDNA-STAMP)

(71) Applicant: CORNELL UNIVERSITY, Ithaca, NY (US)

(72) Inventors: Zhenglong GU, Ithaca, NY (US); Xiaoxian GUO, Ithaca, NY (US); Yiqin WANG, Ithaca, NY (US); Ruoyu ZHANG, White Plains (US)

(73) Assignee: CORNELL UNIVERSITY, Ithaca, NY (US)

(21) Appl. No.: 17/760,652

(22) PCT Filed: Sep. 16, 2020
(86) PCT No.: PCT/US2020/051012

§ 371 Date: Mar. 15, 2022

Related U.S. Application Data
(60) Provisional application No. 62/900,882, filed on Sep. 16, 2019.

(51) Int. Cl. C12Q 1/6883

(52) U.S. Cl. CPC C12Q 1/6883; C12Q 2600/16; C12Q 2600/156; C12Q 1/6869

(57) ABSTRACT

The present disclosure is directed to probe sets for sequencing a mitochondrial genomic DNA, methods of sequencing a mitochondrial DNA using the probe sets, and methods of designing probe sets for sequencing a mitochondrial genomic DNA.

### CROSS REFERENCE TO RELATED APPLICATION

This application claims the benefit of priority from U.S. Provisional Application No. 62/900,882, filed Sep. 16, 2019, the entire contents of which are incorporated herein by reference.

### STATEMENT REGARDING FEDERALLY SPONSORED RESEARCH OR DEVELOPMENT

This invention was made with government support under Grant Nos. R01AI085286, awarded by the National Institutes of Health. The government has certain rights in the invention.

### INCORPORATION BY REFERENCE OF SEQUENCE LISTING

The Sequence Listing in an ASCII text file, named as 37747WO_7738-02_SequenceListing of 20 KB, created on Sep. 14, 2020, and submitted to the United States Patent and Trademark Office via EFS-Web, is incorporated herein by reference.

### BACKGROUND

Mitochondrial diseases are a group of disorders caused by dysfunctional mitochondria, the organelles that generate energy for the cell. Some mitochondrial diseases are caused by mutations in the mitochondrial DNA that affect mitochondrial function. Therefore, there is a need for an easy, low cost, and highly sensitive assay to detect mutations in the mitochondrial DNA.

The human mitochondrial genome (mtDNA) is a circular genome encapsulated in the inner membrane of mitochondria. It encodes 22 tRNA and 2 rRNA genes used for mitochondrial protein synthesis as well as 13 evolutionarily conserved proteins in four of the five mitochondrial oxidation phosphorylation (OXPHOS) protein complexes. The human mitochondrial DNA (mtDNA) has been completely sequenced and is approximately 16,569 base pairs in length. The strands of mtDNA are characterized as “heavy strand” or “light strand” based on their buoyant densities during separation in cesium chloride gradients, which was found to be related to the relative amount of purine (A and G) nucleotide content of the strand.

Deep sequencing studies have shown that mtDNA mutations are much more prevalent in human tissues than previously thought. Given the multicopy nature of mtDNA in a single cell, mtDNA mutations can arise and co-exist with the wild-type allele in a state called heteroplasmy. mtDNA heteroplasmies can increase in fraction through clonal expansion in cells and tissues, without affecting mitochondrial function until their abundance reaches a certain threshold. At an intermediate fraction, a single disease-causing mtDNA mutation may lead to mitochondrial morphological changes and decreased transcription of mtDNA, recapitulating the mild mitochondrial dysfunction in diseases like diabetes and autism. At a relatively high fraction, it may induce global changes of gene expression involved in signal transduction, epigenomic regulation, and pathways implicated in neurodegenerative diseases. Accordingly, the varying fraction and abundance of mtDNA mutations, as well as their tissue sources, may give rise to distinct downstream phenotypes, which poses a challenge for mtDNA studies.

In addition to mtDNA sequence variations, the total number of mtDNA molecules in a cell, known as mtDNA copy number (i.e. mtDNA content), can also impact mitochondrial function. Altered mtDNA content in peripheral tissues is frequently reported in patients with neuropsychiatric disorders, and has been shown to be affected by stressful life events. Recently, several large-scale prospective studies found correlations between low mtDNA content in blood and age-related chronic diseases, such as cardiovascular diseases, illustrating that mtDNA content can serve as a biomarker for age-related decline of mitochondrial function, and a predictor for adverse health outcomes in humans.

Lately, large-scale population studies on human mtDNA have been facilitated by widely available datasets from genome-wide sequencing projects. Off-target reads from whole-exome sequencing (WES) studies can be used to assess mtDNA sequence variations and medium-fraction heteroplasmies. Likewise, whole-genome sequencing (WGS) with deep and uniform coverage on mtDNA can be used to measure mtDNA copy number and low-fraction heteroplasmies in tissues. However, WES and WGS are not cost-effective for investigators to study mtDNA with a large sample size. Even if the genomic datasets have already been produced, analyses of mtDNA are often restricted by their original study design, which rarely allow investigators to study important characteristics of mtDNA, such as the temporal and tissue dynamics of mtDNA heteroplasmies and content.

mtDNA-targeted sequencing is an alternative to genome-wide methods. The main strategy is to isolate and enrich mtDNA from the total genomic background, and thus focus the sequencing capacity on mtDNA reads. These methods normally start with PCR amplification using specific primers and DNA polymerases to amplify mtDNA. Alternatively, mtDNA sequencing libraries can be enriched from total genomic sequencing libraries by using hybridization capture baits derived from mtDNA. Sequencing libraries containing short mtDNA fragments and adaptors are subsequently generated from the PCR products by using commercially available sequencing kits. However, as these library preparation protocols are optimized for processing large, linear genomic DNA, their use for the short 16.6 kb circular mtDNA dramatically increase the overall cost of mtDNA sequencing. To overcome this limitation, Nunez et al. developed a method to add sequencing adaptors directly to the short DNA fragments with T4 DNA ligase, after PCR amplification of mtDNA (PLoS One, 11, e0160958). Although this method can be applied to human mtDNA at a low cost, it has drawbacks that are similar to other methods, since mtDNA enrichment and library construction involve multiple steps and reaction plates, which incurs extra labor and increases the possibility of sample contamination during DNA purification and transferring.

Moreover, most of these mtDNA-enrichment strategies depend on high numbers of PCR cycles to increase mtDNA content, which inevitably introduce errors and artifacts during DNA amplification. These errors can lead to false discovery of mtDNA heteroplasmies, as most of them are of low fraction at a tissue level. A previous study showed that even at a sequencing coverage as high as 20000-fold (X) on mtDNA, the majority of the mtDNA sites were polymorphic with a variant allele fraction (VAF) of ≥0.1%, most of which could result from PCR or sequencing errors.

Other methods that, conversely, reduce the level of the nuclear genome by using exonuclease V to digest the linear nuclear DNA, or by isolating mitochondria with differential centrifugation or magnetic beads for immunoprecipitation require a large amount of DNA input, which is not suited for large-scale population studies with limited DNA sources or frozen tissue biospecimens. Importantly, by processing only mtDNA, the mtDNA-targeted sequencing methods lose valuable information on mtDNA levels in relation to nuclear DNA in the sample, making them unable to quantify mtDNA content in the same assay. Therefore, a cost-effective, accurate and flexible mtDNA sequencing method is urgently needed, especially for studying mtDNA variations in large populations.

### SUMMARY OF THE DISCLOSURE

An aspect of the disclosure us directed to a probe set comprising a first probe subset comprising a plurality of probe pairs and a second probe subset comprising a plurality of probe pairs, wherein each probe pair within each probe subset comprises a ligation probe and an extension probe, wherein each probe pair in the first probe subset comprises probes that anneal to the heavy strand of a mitochondrial genomic DNA and each probe pair in the second probe subset comprises probes that anneal to the light strand of a mitochondrial genomic DNA, wherein each probe pair defines a target region of the mitochondrial genomic DNA that is not identical to any other target region defined by any other probe pair, wherein the target regions defined by the first probe subset and the target regions defined by the second probe subset in combination cover the entirety of the mitochondrial genomic DNA, wherein each ligation probe comprises a first primer annealing sequence and a 5′-phosphorylated ligation arm that is substantially complementary to a first end of the target region on the mitochondrial genomic DNA defined by the probe pair, wherein each extension probe comprises an extension arm that is substantially complementary to a second end of the target region on the mitochondrial genomic DNA defined by the probe pair, and a second primer annealing sequence, and wherein the ligation arm does not anneal to an identical or overlapping sequence on the mitochondrial genomic DNA with the extension arm.

In some embodiments, the probe pairs in the probe subsets are designed such that neighboring target regions in the heavy strand defined by the probe pairs in the first probe subset overlap with neighboring complementary target regions in the light strand defined by the probe pairs in the second probe subset.

In some embodiments, a target region in the heavy strand defined by a probe pair from the first probe subset is followed by an overlapping target region in the light strand defined by a probe pair from the second probe subset.

In some embodiments, each probe pair anneals to a target region that is between 200-600 nucleotides, 300-500 nucleotides, or 399-449 nucleotides in length.

In some embodiments, all the ligation probes comprise a common nucleotide sequence for the first primer annealing sequence, wherein all the extension probes comprise a common nucleotide sequence for the second primer annealing sequence, and wherein the nucleotide sequences of the first primer annealing sequence and the second primer annealing sequence are different.

In some embodiments, (i) each ligation probe further comprises a molecular tag sequence, wherein the molecular tag sequence is unique for each ligation probe; (ii) each extension probe further comprises a molecular tag sequence, wherein the molecular tag sequence is unique for each extension probe; or (iii) each ligation probe further comprises a first molecular tag sequence and each extension probe further comprises a second molecular tag sequence, wherein the first molecular tag sequence is unique for each ligation probe, wherein the second molecular tag sequence is different unique for each ligation probe, and wherein the first molecular tag sequence and the second molecular tag sequence are different from each other.

In some embodiments, each molecular tag sequence is between 10 and 25 nucleotides in length.

Another aspect of the disclosure is directed to a method for sequencing a mitochondrial genomic DNA comprising contacting a sample comprising a denatured mitochondrial genomic DNA with the probe set of the instant disclosure (as defined above and in the detailed description) under conditions to permit the probe set to hybridize to the mitochondrial genomic DNA wherein the probe set comprises a first probe subset comprising a plurality of probe pairs and a second probe subset comprising a plurality of probe pairs, wherein each probe pair within each probe subset comprises a ligation probe and an extension probe, wherein each probe pair in the first probe subset comprises probes that anneal to the heavy strand of a mitochondrial genomic DNA and each probe pair in the second probe subset comprises probes that anneal to the light strand of a mitochondrial genomic DNA, wherein each probe pair defines a target region of the mitochondrial genomic DNA that is not identical to any other target region defined by any other probe pair, wherein the target regions defined by the first probe subset and the target regions defined by the second probe subset in combination cover the entirety of the mitochondrial genomic DNA, wherein each ligation probe comprises a first primer annealing sequence and a 5′-phosphorylated ligation arm that is substantially complementary to a first end of the target region on the mitochondrial genomic DNA defined by the probe pair, wherein each extension probe comprises an extension arm that is substantially complementary to a second end of the target region on the mitochondrial genomic DNA defined by the probe pair, and a second primer annealing sequence, and wherein the ligation arm does not anneal to an identical or overlapping sequence on the mitochondrial genomic DNA with the extension arm; performing an enzymatic gap filling reaction to connect the ligation probe and the extension probe in each pair of probes, thereby producing a ligation product; amplifying the ligation product; and sequencing the amplified products.

In some embodiments, the amplifying step is achieved using a first primer that anneals to the first primer annealing sequence and a second primer that anneals to the complementary strand of the second primer annealing sequence.

In some embodiments, the sequencing is performed using next-generation sequencing.

In some embodiments, the probe pairs in the probe subsets are designed such that neighboring target regions in the heavy strand defined by the probe pairs in the first probe subset overlap with neighboring complementary target regions in the light strand defined by the probe pairs in the second probe subset.

In some embodiments, a target region in the heavy strand defined by a probe pair from the first probe subset is followed by an overlapping target region in the light strand defined by a probe pair from the second probe subset.

In some embodiments, each probe pair anneals to a target region that is between 200-600 nucleotides, 300-500 nucleotides, or 399-449 nucleotides in length.

In some embodiments, all the ligation probes comprise a common nucleotide sequence for the first primer annealing region, wherein all the extension probes comprise a common nucleotide sequence for the second primer annealing region, and wherein the nucleotide sequence of the first primer annealing region and the nucleotide sequence of the second primer annealing region are different.

In some embodiments, (i) each ligation probe further comprises a molecular tag sequence, wherein the molecular tag sequence is different unique for each ligation probe; (ii) each extension probe further comprises a molecular tag region, wherein the molecular tag sequence is different unique for each extension probe; or (iii) each ligation probe further comprises a first molecular tag sequence and each extension probe further comprises a second molecular tag sequence, wherein the first molecular tag sequence is different unique for each ligation probe, wherein the second molecular tag sequence is different unique for each ligation probe, and wherein the first molecular tag sequence and the second molecular tag sequence are different from each other.

In some embodiments, each molecular tag sequence is between 10 and 25 nucleotides in length.

In some embodiments, the method further comprises removing from sequencing reads sequences of the primer annealing regions, thereby producing trimmed reads; aligning the trimmed reads based on the molecular tag regions, wherein aligned reads with identical molecular tag regions represent PCR duplicates from one probe pair and aligned reads with different molecular tag regions represent an overlapping region from different probe pairs; and determining whether a mutation exists in the aligned trimmed reads; and when a mutation is detected, classifying the mutation as a true variant when the mutation is found in all members of aligned reads with identical molecular tag regions, and classifying the mutation as an error (for example, a PCR error or a sequencing error) when the mutation is not found in all members of aligned reads with identical molecular tag regions.

In some embodiments, the sample is from a subject having or suspected of having a mitochondrial disease selected from the group consisting of MELAS (Mitochondrial encephalopathy, lactic acidosis, and stroke-like episodes Syndrome), NARP (Neuropathy, ataxia, and retinitis pigmentosa), Leigh's Syndrome, MERRF (myoclonic epilepsy with ragged red fibers) Syndrome, Leber's hereditary optic neuropathy (LHON), Kern-Sayre Syndrome, Mitochondrial neurogastrointestinal encephalopathy syndrome (MNGIE), and Aplers Disease.

In some embodiments, the sample is from a Huntington's Disease patient.

Another aspect of the disclosure is directed to a method for designing a probe set for sequencing a mitochondrial genomic DNA comprising designing a probe set comprising a first probe subset comprising a plurality of probe pairs and a second probe subset comprising a plurality of probe pairs, wherein each probe pair within each probe subset comprises a ligation probe and an extension probe, wherein each probe pair in the first probe subset comprises probes that anneal to the heavy strand of a mitochondrial genomic DNA and each probe pair in the second probe subset comprises probes that anneal to the light strand of a mitochondrial genomic DNA, wherein each probe pair defines a target region of the mitochondrial genomic DNA that is not identical to any other target region defined by any other probe pair, wherein the target regions defined by the first probe subset and target regions defined by the second probe subset in combination cover the entirety of the mitochondrial genomic DNA, wherein each ligation probe comprises a first primer annealing region and a 5′-phosphorylated ligation arm that is substantially complementary to a first end of the target region on the mitochondrial genomic DNA defined by the probe pair, wherein each extension probe comprises an extension arm that is substantially complementary to a second end of the target region on the mitochondrial genomic DNA defined by the probe pair, a molecular tag region, and a second primer annealing region, and wherein the ligation arm does not anneal to an identical or overlapping sequence on the mitochondrial genomic DNA with the extension arm.

In some embodiments, the probe pairs in the probe subsets are designed such that the target regions in the heavy strand defined by the probe pairs in the first probe subset overlap with complementary target regions in the light strand defined by the probe pairs in the second probe subset.

In some embodiments, a target region in the heavy strand defined by a probe pair from the first probe subset is followed by an overlapping target region in the light strand defined by a probe pair from the second probe subset.

In some embodiments, each probe pair anneals to a target region that is between 200-600 nucleotides, 300-500 nucleotides, or 399-449 nucleotides in length.

In some embodiments, all the ligation probes comprise a common nucleotide sequence for the first primer annealing region, wherein all the extension probes comprise a common nucleotide sequence for the second primer annealing region, and wherein the nucleotide sequence of the first primer annealing region and the nucleotide sequence of the second primer annealing region are different.

In some embodiments, (i) each ligation probe further comprises a molecular tag sequence, wherein the molecular tag sequence is different unique for each ligation probe; (ii) each extension probe further comprises a molecular tag region, wherein the molecular tag sequence is different unique for each extension probe; or (iii) each ligation probe further comprises a first molecular tag sequence and each extension probe further comprises a molecular tag sequence, wherein the first molecular tag sequence is different unique for each ligation probe, wherein the second molecular tag sequence is different unique for each ligation probe, and wherein the first molecular tag sequence and the second molecular tag sequence are different from each other.

In some embodiments, each molecular tag sequence is between 10 and 25 nucleotides in length.

Yet another aspect of the disclosure is directed to a method of determining the mitochondrial mutation load in a subject comprising contacting a sample comprising a denatured mitochondrial genomic DNA with a probe set wherein the probe set comprises: a first probe subset comprising a plurality of probe pairs and a second probe subset comprising a plurality of probe pairs, wherein each probe pair within each probe subset comprises a ligation probe and an extension probe, wherein each probe pair in the first probe subset comprises probes that anneal to the heavy strand of a mitochondrial genomic DNA and each probe pair in the second probe subset comprises probes that anneal to the light strand of a mitochondrial genomic DNA, wherein each probe pair defines a target region of the mitochondrial genomic DNA that is not identical to any other target region defined by any other probe pair, wherein the target regions defined by the first probe subset and the target regions defined by the second probe subset in combination cover the entirety of the mitochondrial genomic DNA, wherein each ligation probe comprises a first primer annealing sequence and a 5′-phosphorylated ligation arm that is substantially complementary to a first end of the target region on the mitochondrial genomic DNA defined by the probe pair, wherein each extension probe comprises an extension arm that is substantially complementary to a second end of the target region on the mitochondrial genomic DNA defined by the probe pair, and a second primer annealing sequence, and wherein the ligation arm does not anneal to an identical or overlapping sequence on the mitochondrial genomic DNA with the extension arm; performing an enzymatic gap filling reaction to connect the ligation probe and the extension probe in each pair of probes, thereby producing a ligation product; amplifying the ligation product; sequencing the amplified product, removing from sequencing reads sequences of the primer annealing regions, thereby producing trimmed reads; aligning the trimmed reads based on the molecular tag regions, wherein aligned reads with identical molecular tag regions represent PCR duplicates from one probe pair and aligned reads with different molecular tag regions represent an overlapping region from different probe pairs; determining whether a mutation exists in the aligned trimmed reads, wherein when a mutation is detected, classifying the mutation as a true variant when the mutation is found in all members of aligned reads with identical molecular tag regions, and classifying the mutation as an error (for example, a PCR error or a sequencing error) when the mutation is not found in all members of aligned reads with identical molecular tag regions; and thereby determining the mitochondrial mutation load in a subject.

In some embodiments, the sequencing is performed using next-generation sequencing.

In some embodiments, the probe pairs in the probe subsets are designed such that neighboring target regions in the heavy strand defined by the probe pairs in the first probe subset overlap with neighboring complementary target regions in the light strand defined by the probe pairs in the second probe subset.

In some embodiments, a target region in the heavy strand defined by a probe pair from the first probe subset is followed by an overlapping target region in the light strand defined by a probe pair from the second probe subset.

In some embodiments, each probe pair anneals to a target region that is between 200-600 nucleotides, 300-500 nucleotides, or 399-449 nucleotides in length.

In some embodiments, all the ligation probes comprise a common nucleotide sequence for the first primer annealing region, wherein all the extension probes comprise a common nucleotide sequence for the second primer annealing region, and wherein the nucleotide sequence of the first primer annealing region and the nucleotide sequence of the second primer annealing region are different.

In some embodiments, (i) each ligation probe further comprises a molecular tag sequence, wherein the molecular tag sequence is different unique for each ligation probe; (ii) each extension probe further comprises a molecular tag region, wherein the molecular tag sequence is different unique for each extension probe; or (iii) each ligation probe further comprises a first molecular tag sequence and each extension probe further comprises a second molecular tag sequence, wherein the first molecular tag sequence is different unique for each ligation probe, wherein the second molecular tag sequence is different unique for each ligation probe, and wherein the first molecular tag sequence and the second molecular tag sequence are different from each other.

In some embodiments, each molecular tag sequence is between 10 and 25 nucleotides in length.

In some embodiments, the subject is a mammal suspected of having a mitochondrial disease.

In some embodiments, the mammal is a human.

In some embodiments, the mitochondrial disease is selected from the group consisting of MELAS (Mitochondrial encephalopathy, lactic acidosis, and stroke-like episodes Syndrome), NARP (Neuropathy, ataxia, and retinitis pigmentosa), Leigh's Syndrome, MERRF (myoclonic epilepsy with ragged red fibers) Syndrome, Leber's hereditary optic neuropathy (LHON), Kern-Sayre Syndrome, Mitochondrial neurogastrointestinal encephalopathy syndrome (MNGIE), Aplers Disease, Huntington's Disease, Alzheimer Disease and cancer.

Another aspect of the disclosure is directed to a method for determining the relative mitochondrial genomic DNA (mtDNA) content comprising denaturing the mtDNA and the nuclear DNA(nDNA) in the sample; capturing a target region of the denatured mtDNA in the sample using the probe set described herein; capturing a target region of the denatured nDNA using at least one nDNA-targeting probe pair, wherein each nDNA-targeting probe pair comprises an nDNA-targeting ligation probe and an nDNA-targeting extension probe; determining the amount of mtDNA and the amount of nDNA; and determining the ratio of the amount of mtDNA versus the amount of nDNA.

In some embodiments, each nDNA-targeting ligation probe comprises a first primer annealing sequence and a 5′-phosphorylated ligation arm that is substantially complementary to a sequence at a first end of a target region on the nuclear genomic DNA defined by the probe pair; each nDNA-targeting extension probe comprises a second primer annealing sequence and an extension arm that is substantially complementary to a sequence at a second end of the target region on the nuclear genomic DNA defined by the probe pair.

In some embodiments, the method further comprises amplifying the captured mtDNA and nDNA.

In some embodiments, the capturing comprises performing an enzymatic gap filling reaction.

In some embodiments, determining the amount of mtDNA and the amount of nDNA is achieved by next generation sequencing or by quantitative Polymerase Chain Reaction (PCR).

Another aspect of the disclosure is directed to a method of determining heteroplasmy in a subject comprising contacting a sample comprising a denatured mitochondrial genomic DNA with a probe set wherein the probe set comprises a first probe subset comprising a plurality of probe pairs and a second probe subset comprising a plurality of probe pairs, wherein each probe pair within each probe subset comprises a ligation probe and an extension probe, wherein each probe pair in the first probe subset comprises probes that anneal to the heavy strand of a mitochondrial genomic DNA and each probe pair in the second probe subset comprises probes that anneal to the light strand of a mitochondrial genomic DNA, wherein each probe pair defines a target region of the mitochondrial genomic DNA that is not identical to any other target region defined by any other probe pair, wherein the target regions defined by the first probe subset and the target regions defined by the second probe subset in combination cover the entirety of the mitochondrial genomic DNA, wherein each ligation probe comprises a first primer annealing sequence and a 5′-phosphorylated ligation arm that is substantially complementary to a first end of the target region on the mitochondrial genomic DNA defined by the probe pair, wherein each extension probe comprises an extension arm that is substantially complementary to a second end of the target region on the mitochondrial genomic DNA defined by the probe pair, and a second primer annealing sequence, and wherein the ligation arm does not anneal to an identical or overlapping sequence on the mitochondrial genomic DNA with the extension arm; performing an enzymatic gap filling reaction to connect the ligation probe and the extension probe in each pair of probes, thereby producing a ligation product; amplifying the ligation product; sequencing the amplified products; removing from sequencing reads sequences of the primer annealing regions, thereby producing trimmed reads; aligning the trimmed reads based on the molecular tag regions, wherein aligned reads with identical molecular tag regions represent PCR duplicates from one probe pair and aligned reads with different molecular tag regions represent an overlapping region from different probe pairs; determining whether heteroplasmy exists in the aligned trimmed reads, wherein when a mutation is detected, classifying the mutation as a heteroplasmy variant when the mutation is found in an overlapping region from different probe pairs; and thereby determining the heteroplasmy in a subject.

In some embodiments, the sequencing is performed using next-generation sequencing.

In some embodiments, the probe pairs in the probe subsets are designed such that neighboring target regions in the heavy strand defined by the probe pairs in the first probe subset overlap with neighboring complementary target regions in the light strand defined by the probe pairs in the second probe subset.

In some embodiments, a target region in the heavy strand defined by a probe pair from the first probe subset is followed by an overlapping target region in the light strand defined by a probe pair from the second probe subset.

### BRIEF DESCRIPTION OF THE DRAWINGS

FIGS. 1A-1B. Design and workflow of STAMP. (A) Schematic diagrams of STAMP for mtDNA sequencing and relative mtDNA content assessment with EL probes. The locations of the 46 mtDNA EL probes are shown with pairs of arrows next to the mitochondrial genome. The locations of the 5 nDNA EL probes are shown with horizontal red lines across chromosomes 1, 8, 14 15 and 19. (B) Schematic diagrams for mtDNA capturing, gap-filling reaction, library construction, read processing, and consensus read calling in STAMP.

FIG. 2. Effective capture of mtDNA with EL probes. The relative depth of coverage of consensus reads on mtDNA for each of the 46 regions captured by EL probes (from A1 to D10). The purple dotted line and red dashed line indicate 50% and 20% of the mean sequence coverage, respectively.

FIGS. 3A-3F. Accurate detection of mtDNA variants in sample mixtures. The numbers of nucleotide mismatches between consensus reads and the reference mtDNA sequence in (A) sample 1 and (C) sample 2. The proportions of variant alleles per base in the consensus reads after filtering out low-quality bases (BAQ<30) and consensus reads with excessive mismatches (NM>5) in (B) sample 1 and (D) sample 2. #PE-reads: the number of paired-end reads used to construct the consensus read. The VAFs of the mtDNA variants detected in the mixtures of sample 1 and sample 2 were depicted in (E) for variants at the 58 polymorphic sites and in (F) for variants at the 5 heteroplasmic sites. Each dotted line in (E) and (F) refers to the VAF changes of one variant in relation to the sample proportion indicated by the values on the x axis. Both x and y axes in (E) are shown on a log scale.

FIGS. 4A-4H. Reliable sequencing of mtDNA in a population study. Results of STAMP sequencing performed on 182 lymphoblast samples of REGISTRY are shown. (A) Median depth of coverage of consensus reads on mtDNA used for calling mtDNA variants. (B and C) Proportions of mtDNA sites with depths of consensus read coverage greater than (B) 0.2 and (C) 0.5 times the mean value, respectively. (D) Proportions of variant alleles per base in the consensus reads used for calling mtDNA variants. (E) Correlations of VAFs of 45 mtDNA heteroplasmies identified in the STAMP replicates performed on 8 samples. (F) Correlations of the relative mtDNA content measured by using STAMP and qPCR. (G) The box plots of mtDNA heteroplasmies detected in lymphoblasts of individuals aged above and under the sample median of 48 years old. (H) The box plots of the relative mtDNA content (STAMP-CN) detected in lymphoblasts of individuals aged above and under the sample median of 48 years old.

FIGS. 5A-5D. Mapping rate and duplication rate of paired-end reads in STAMP. The results were estimated based on paired-end (PE) reads and consensus reads from 182 lymphoblast samples of REGISTRY. The distributions of the percentage of paired-end reads mapped to the EL-probe-targeted regions in (A) mtDNA and (B) nDNA were depicted using histograms. The distributions of the average numbers of consensus reads constructed from increasing numbers of paired-end reads were depicted in (C) for mtDNA-probe-targeted regions and in (D) for nDNA-probe-targeted regions. Error bars in (C) and (D) represent the interquartile range.

FIGS. 6A-6F. Power of detecting mtDNA heteroplasmies by using STAMP. The average numbers of consensus reads with and without duplication in (A), (C) and (E) were estimated based on a Poisson distribution with the numbers of consensus reads and paired-end reads. The corresponding error rates of STAMP were computed based on the proportions of variant alleles per base in the consensus reads constructed with and without duplication shown in FIG. 4D. The statistical power to discriminate real heteroplasmies of varying VAFs from errors, at 16569 sites of mtDNA, was estimated using one-tailed power calculation for one sample proportion. The statistical power was computed with the error rates and the numbers of consensus reads indicated in the legends of panels (B), (D) and (F). The statistical power with 50% and 20% of the average number of consensus reads was also depicted for the low-coverage regions in mtDNA. The related results are shown in (A) and (B) for detecting high/medium-fraction heteroplasmies, in (C) and (D) for detecting medium/low-fraction heteroplasmies, and in (E) and (F) for detecting very-low-fraction heteroplasmies.

FIG. 7. Four modules of STAMP toolkit.

FIG. 8. Study flow chart. This study flow chart summarizes the lymphoblast and blood samples of REGISTRY used for mtDNA analyses.

FIGS. 9A-9B. mtDNA variant incidence in lymphoblasts of HD patients and control individuals. The results are shown in (A) for predicted pathogenic heteroplasmies and in (B) for all mtDNA heteroplasmies. The values on the x axes refer to the minimum VAFs of the heteroplasmies used in the analyses, from a low fraction at 1% to a high fraction at 30%. The bars represent the average numbers of heteroplasmies ±SEM. The P values for mtDNA heteroplasmies from the logistic regression analyses of the disease status are shown above the bars. The effects of mtDNA heteroplasmies, as odds ratios for HD, are illustrated with the green lines indicated by the values on the green y axes on a logarithmic scale.

FIGS. 10A-10B. mtDNA variant dosages and pathogenicity in lymphoblasts of HD patients and control individuals. (A) Bar plots of the average variant dosages of predicted pathogenic heteroplasmies. The P values for mtDNA variant dosages from the logistic regression analyses of disease status are indicated above the bars representing the corresponding HD stages. In the linear regression analyses of disease stages, HD stages were treated as a continuous dependent variable with integer values from 1 to 5. NA: not applicable. (B) The average pathogenicity of nonsynonymous heteroplasmies in increasing VAF categories indicated in the legend. The Pearson's correlation between the heteroplasmic VAF and the pathogenicity score, as well as the corresponding P value, are shown in each panel of (B). The CADD scores are shown with the inverse normal transformed values, which increase with the chance of a heteroplasmy being pathogenic. The red lines in B represent the fitted regression lines for the VAF categories and the pathogenicity scores. NA: not applicable. Error bars in A and B represent SEM.

FIGS. 11A-11D. Associations of pathogenic mtDNA variant dosages with HD clinical phenotypes and genetic burden. The pathogenic mtDNA variant dosages were computed using either heteroplasmies with medium or high pathogenicity or heteroplasmies with only high pathogenicity. The significance levels of the associations of pathogenic mtDNA variant dosages with HD clinical phenotypes are shown in (A) for UHDRS total functional capacity score, in (B) for total motor score, and in (C) for symbol digit modalities test score, all of which were assessed with adjustment for CAG repeat length. The significance levels of the associations with HD genetic burden are shown in (D) for normalized CAG-age product, which were assessed without adding CAG repeat length and age as covariates. The mean±SEM of the phenotypes in the lymphoblasts with low (<0.05), medium-to-high (0.05-0.3), and high pathogenic mtDNA variant dosages (>0.3) are illustrated in each panel.

FIGS. 12A-12C. mtDNA variant incidence and fraction changes detected in longitudinal blood samples of HD patients. (A) Bar plots of the incidence of mtDNA heteroplasmies detected in the baseline and follow-up blood samples. The incidence of heteroplasmies in different VAF categories was depicted using colors indicated in the legend. The P value from paired t-test of the overall heteroplasmy incidence is shown. (B) Venn Diagram of mtDNA heteroplasmies detected in samples from the same individuals. The light blue cycle represents heteroplasmies identified in the baseline sample. The red cycle represents heteroplasmies identified in the follow-up samples. The overlapping region shows the share of 508 mtDNA heteroplasmies with VAF≥0.2% in both samples. (C) Histogram and box plots of the distribution of the VAF changes of the 508 shared mtDNA heteroplasmies during the follow-up.

FIGS. 13A-13C. Changes of mtDNA variant fractions and pathogenicity in blood during HD progression. (A) Box plots of the VAF changes of pre-existing mtDNA heteroplasmies in blood samples of HD patients with and without a progression of disease stage during the follow-up. The P values from t-test and Cohen's d are shown for the difference between the patient groups, which were computed using either all heteroplasmies, heteroplasmies with medium or high pathogenicity, or heteroplasmies with only high pathogenicity. Each red dot in A indicates one heteroplasmy with its VAF change during the follow-up indicated by the value on the y axis. (B and C) The correlations between the VAF changes of pre-existing nonsynonymous heteroplasmies and their CADD pathogenicity scores among (B) stable-stage patients and among (C) progressed-stage patients. The CADD scores are shown with the inverse normal transformed values, which increase with the chance of a heteroplasmy being pathogenic. The dashed lines represent the fitted regression lines.

FIGS. 14A-14C. Base changes of mtDNA heteroplasmies detected in lymphoblasts and blood samples. The proportions of different types of base changes are shown for the heteroplasmies detected in lymphoblasts of (A) HD patients and (B) control individuals, and in (C) blood samples of HD patients.

### DETAILED DESCRIPTION

### Definitions

As used herein, the term “about” refers to an approximately +/−10% variation from a given value.

The term “amplification” or “amplify” as used herein includes methods for copying a target nucleic acid, thereby increasing the number of copies of a selected nucleic acid sequence. Amplification may be exponential or linear. A target nucleic acid may be either DNA or RNA. The regions or sequences of a target nucleic acid amplified in this manner form an “amplicon” or “amplification product”. While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols (1990), Innis et al., Eds., Academic Press, San Diego, Calif., pp 13-20; Wharam, et al., Nucleic Acids Res. (2001), June 1; 29(11):E54-E54; Hafner, et al., Biotechniques (2001), 4:852-6, 858, 860.

The term “capture” or “capturing” refers to making a copy of a target region of a nucleic acid defined by two probes. The number of “captured” copies of a target region of a nucleic acid is the same as the number of copies of the target region and proportional to the number of copies/amount of nucleic acid. In some embodiments, the nucleic acid is mitochondrial genomic DNA. In these instances, as there are multiple mitochondria in one cell, there are multiple copies of mtDNA (and in combination a target region as well). Therefore, the number of copies of a captured target region is indicative of/proportional to the amount of mtDNA, and thus the number of mitochondria. In some embodiments, the captured nucleic acid is nuclear genomic DNA. In some embodiments, “capturing” is achieved by enzymatic gap filling.

The term “DNA,” as used herein, refers to a nucleic acid molecule of one or more nucleotides in length, wherein the nucleotide(s) are nucleotides. By “nucleotide” it is meant a naturally-occurring nucleotide, as well modified versions thereof. The term “DNA” includes double-stranded DNA, single-stranded DNA, isolated DNA such as cDNA, as well as modified DNA that differs from naturally-occurring DNA by the addition, deletion, substitution and/or alteration of one or more nucleotides as described herein.

The term “gene,” as used herein, refers to a segment of nucleic acid that encodes an individual protein or RNA and can include both exons and introns together with associated regulatory regions such as promoters, operators, terminators, 5′ untranslated regions, 3′ untranslated regions, and the like.

The term “insertion” (or “insertion mutation”), as used herein, refers to the addition of one or more nucleotides into a nucleic acid sequence (e.g., into a wild type or normal nucleic acid sequence). Insertions mutations can differ in the number of nucleotides inserted, or the nature or identity of nucleotides inserted.

The term “mitochondrial disease” refers to a group of disorders caused by dysfunctional mitochondria, as well as disorders that dysfunctional mitochondria contribute to and/or exacerbate the disease progression. In some embodiments, the term “mitochondrial disease” includes classic mitochondrial dysfunction diseases such as MELAS (Mitochondrial encephalopathy, lactic acidosis, and stroke-like episodes Syndrome), NARP (Neuropathy, ataxia, and retinitis pigmentosa), Leigh's Syndrome, MERRF (myoclonic epilepsy with ragged red fibers) Syndrome, Leber's hereditary optic neuropathy (LHON), Kern-Sayre Syndrome, Mitochondrial neurogastrointestinal encephalopathy syndrome (MNGIE), and Aplers Disease, and other diseases such as Huntington's Disease (HD), Alzheimer Disease (AD) and cancer.

A mutation is meant to encompass at least a nucleotide variation in a sequence relative to a wild type or normal sequence. A mutation may include a substitution, a deletion, an inversion or an insertion. With respect to an encoded polypeptide, a mutation may be “silent” and result in no change in the encoded polypeptide sequence, or a mutation may result in a change in the encoded polypeptide sequence. For example, a mutation may result in a substitution in the encoded polypeptide sequence. A mutation may result in a frameshift with respect to the encoded polypeptide sequence.

The term “percent (%) sequence identity,” as used herein with respect to a reference sequence is defined as the percentage of nucleotides in a candidate sequence that are identical with the nucleotides in the reference polynucleotide sequence over the window of comparison after optimal alignment of the sequences and introducing gaps, if necessary, to achieve the maximum percent sequence identity.

As used herein, the term “primer” refers to an oligonucleotide, whether occurring naturally as in a purified restriction digest or produced synthetically, which is capable of acting as a point of initiation of nucleic acid sequence synthesis when placed under conditions in which synthesis of a primer extension product which is complementary to a nucleic acid strand is induced, i.e. in the presence of different nucleotide triphosphates and a polymerase in an appropriate buffer (“buffer” includes pH, ionic strength, cofactors etc.) and at a suitable temperature. One or more of the nucleotides of a primer can be modified for instance by addition of a methyl group, a biotin or digoxigenin moiety, a fluorescent tag or by using radioactive nucleotides. A primer sequence need not reflect the exact sequence of a template. For example, a non-complementary nucleotide fragment may be attached to the 5′ end of a primer, with the remainder of the primer sequence being substantially complementary to the complementary strand of a template. The term “primer” as used herein includes all forms of primers that may be synthesized including peptide nucleic acid primers, locked nucleic acid primers, phosphorothioate modified primers, labeled primers, and the like. Primers can be at least about 10, 15, 20, 25, 30, 35, 40, 45, 50, or more nucleotides in length; typically, a primer has a length of 10, 11, 12, 13, 14, 15, 16, 17, 18, 19, 20, 21, 22, 23, 24, 25, 26, 27, 28, 29, or 30 nucleotides. An optimal length for a particular primer application may be readily determined in the manner described in H. Erlich, PCR Technology, Principles and Application for DNA Amplification (1989). Primers can be labeled with a detectable molecule or substance, such as a fluorescent molecule, a radioactive molecule or any other labels known in the art. Labels are known in the art that generally provide (either directly or indirectly) a signal. The term “labeled” is intended to encompass direct labeling of the probe and primers by coupling (i.e., physically linking) a detectable substance as well as indirect labeling by reactivity with another reagent that is directly labeled. Examples of detectable substances include but are not limited to radioactive agents or a fluorophore (e.g. fluorescein isothiocyanate (FITC), phycoerythrin (PE), cyanine (Cy3), VIC fluorescent dye, FAM (6-carboxyfluorescein) or Indocyanine (Cy5)).

A “probe” refers to a nucleic acid that interacts with a target nucleic acid via hybridization. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases and modified sugar moieties. In some embodiments, a probe comprises between 15 and 120 nucleotides. In some embodiments, a probe comprises about 15, 20, 25, 30, 35, 40, 45, 50, 55, 60, 65, 70, 75, 80, 85, 90, 95, 100, 105, 110 or 120 nucleotides. In some embodiments, a probe may be fully complementary to a target nucleic acid sequence or partially complementary. A probe may include a primer sequence that can initiate a nucleic acid polymerization reaction (e.g. a PCR reaction). A probe may also function as a primer for a PCR reaction or an enzymatic gap filling reaction. Probes can be labeled or unlabeled, or modified in any of a number of ways well known in the art.

The following terms are used herein to describe the sequence relationships between two or more polynucleotide molecules: “reference sequence,” “window of comparison,” “sequence identity,” “percent (%) sequence identity,” and “substantial identity.” A “reference sequence” is a defined sequence used as a basis for a sequence comparison; a reference sequence may be a subset of a larger sequence, for example, as a segment of a full-length cDNA or gene, or may comprise a complete cDNA or gene sequence. Generally, a reference polynucleotide sequence is at least 20 nucleotides in length, and often at least 50 nucleotides in length.

The term “selectively hybridize” or “specifically hybridize” or “anneal,” as used herein, refers to the ability of a particular nucleic acid sequence to bind specifically to a target nucleic acid sequence. Selective hybridization generally takes place under hybridization and wash conditions that minimize appreciable amounts of detectable binding to non-specific nucleic acids. High stringency conditions can be used to achieve selective hybridization and are known in the art and discussed herein. Typically, hybridization and washing conditions are performed at high stringency according to conventional hybridization procedures with washing conditions utilizing a solution comprising 1-3×SSC, 0.1-1% SDS at 50-70° C., optionally with a change of wash solution after about 5-30 minutes. For instance, in the present disclosure, a nucleic acid sequence is considered to selectively hybridize to a target sequence if the nucleic acid sequence specifically anneals to the target sequence under PCR reaction conditions, e.g., in a reaction mixture comprising dNTPs, DNA polymerase and a PCR buffer comprising Mg²⁺ at a temperature typically in the range of 55-60° C. Nucleic acid sequences (e.g., primers, probes, probe regions (e.g., extension or ligation arms)) having significant sequence identity to the complement of a target sequence is expected to selectively hybridize or anneal to the target sequence. Nucleic acid sequences with at least 80% sequence identity, and at least 90%, 95%, 98% or 99% sequence identity as compared to a complement of a reference sequence over a window of comparison are considered to have significant or substantial sequence identity with the reference sequence. Similarly, the phrase “substantially complementary” refers to a nucleic acid sequence with at least 80% sequence identity, and at least 90%, 95%, 98% or 99% sequence identity as compared to a complement of a reference sequence over a window of comparison.

The term “sequence identity” means that two polynucleotide sequences are identical (i.e., on a nucleotide-by-nucleotide basis) over the window of comparison.

The term “wild-type” refers to a gene or a gene product that has the characteristics of that gene or gene product when isolated from a naturally occurring source. A wild-type gene is that which is most frequently observed in a population and is thus arbitrarily designated as the “normal” or “wild-type” form of die gene. “Wild-type” may also refer to the sequence at a specific nucleotide position or positions, or the sequence at a particular codon position or positions, or the sequence at a particular amino acid position or positions. As used herein, “mutant” “modified” or “polymorphic” refers to a gene or gene product which displays modifications in sequence and or functional properties (i.e., altered characteristics) when compared to the wild-type gene or gene product. The term “mutant” “modified” or “polymorphic” also refers to the sequence at a specific nucleotide position or positions, or the sequence at a particular codon position or positions, or the sequence at a particular amino acid position Or positions.

The term “subject” refers to a mammal having or suspected of having a mitochondrial genomic DNA-related disease (a mitochondrial disorder”). In some embodiments, the subject is a human. In some embodiments, the subject is a domesticated animal such as a cat, a dog, a cow, a sheep, a goat, a donkey and a horse.

A “window of comparison”, as used herein, refers to a conceptual segment of the reference sequence of at least 15 contiguous nucleotide positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A.) (1988) 85:2444), using computerized implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e., resulting in the highest percentage of identity over the comparison window) is then selected.

The present disclosure is directed to probe sets for sequencing a mitochondrial genomic DNA, methods of sequencing a mitochondrial DNA using the probe sets, and methods of designing probe sets for sequencing a mitochondrial genomic DNA.

### Probe Sets

An aspect of the disclosure is directed to a probe set for sequencing a mitochondrial genomic DNA. In some embodiments the probe set comprises a first probe subset comprising a plurality of probe pairs and a second probe subset comprising a plurality of probe pairs. As used in this disclosure, the phrase “plurality of probe pairs” refers to at least 5, least 10, at least 12, at least 15, at least 20, at least 25, or at least 30 probe pairs in each probe subset. In a specific embodiment, the phrase “plurality of probe pairs” refers to 23, 24 or 25 probe pairs in each probe subset.

In some embodiments, each probe pair within each probe subset comprises a ligation probe and an extension probe wherein the ligation probe of the probe pair has a different nucleic acid sequence than the extension probe of the same probe pair. In some embodiments, each probe pair in the first probe subset comprises probes (i.e., ligation probe and extension probe pairs) that specifically hybridize to sequences in the heavy strand of a mitochondrial genomic DNA, and each probe pair in the second probe subset comprises probes (i.e., ligation probe and extension probe pairs) that specifically hybridize to sequences in the light strand of a mitochondrial genomic DNA. In some embodiments, the ligation probe and the extension probe of a probe pair specifically hybridize to sequences that are at least 200 nucleotides, but no more than 600 nucleotides, apart on the same strand of the mitochondrial genomic DNA. The sequence between the ligation probe and the extension probe of a probe pair is said to be “captured” or “defined” by the probe pair. The sequence between the ligation probe and the extension probe of a probe pair is also called the “target region” of the probe pair. In some embodiments, the probes in a probe pair capture or define a target region that is between 200-600 nucleotides, between 300-500 nucleotides, or between 399-449 nucleotides in length. In some embodiments, each probe pair captures (or “defines”) a target region that is about 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 nucleotides long.

In some embodiments, each ligation probe comprises a first primer annealing sequence and a 5′-phosphorylated ligation arm that is substantially complementary to a sequence at a 5′ border (a first end) of a target region on the mitochondrial genomic DNA defined by the probe pair. In some embodiments, the ligation arm comprises between 15 and 45 nucleotides. In some embodiments, the ligation arm is about 15, 18, 20, 25, 28, 30, 35, 38, 40, or 45 nucleotides long. In some embodiments, the ligation arm is at least 15, 18, 20, 25, 28, 30, or 35 nucleotides long, but is no longer than 80, 70, 60, or 50 nucleotides.

In some embodiments, each extension probe comprises a second primer annealing sequence and an extension arm that is substantially complementary to a sequence at a 3′ border (a second end) of the target region on the mitochondrial genomic DNA defined by the probe pair. In some embodiments, the extension arm comprises between 15 and 45 nucleotides. In some embodiments, the extension arm is about 15, 18, 20, 25, 28, 30, 35, 38, 40, or 45 nucleotides long. In some embodiments, the extension arm is at least 15, 18, 20, 25, 28, 30, or 35 nucleotides long, but is no longer than 80, 70, 60, or 50 nucleotides.

In a specific embodiment, the target region is about 300-500 nucleotides long (i.e., the ligation probe and the extension probe of a probe pair specifically hybridize to sequences that are about 300-500 nucleotides apart), and the ligation arm of the ligation probe that specifically hybridizes to the 5′ border (first end) of the target region is between 15-35 nucleotides long and the extension arm of the extension probe that specifically hybridizes to the 3′ border (second end) of the target region is between 15-35 nucleotides long.

In some embodiments, each probe pair (comprised of a ligation probe and an extension probe) defines a target region of the mitochondrial genomic DNA that is not identical to any other target region defined by any other probe pair. In some embodiments, the target regions defined by the first probe subset and the target regions defined by the second probe subset in combination cover the entirety of the mitochondrial genomic DNA.

In some embodiments, the ligation arm does not anneal (specifically hybridize) to an identical or overlapping sequence on the mitochondrial genomic DNA with the extension arm.

In a specific embodiment, the ligation arm of the ligation probe and the extension arm of the extension probe of a probe pair are selected from the pairs recited in Table 4 (i.e., selected from the mt-DNA-specific pairs shown by SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, SEQ ID NOs: 34 and 35, SEQ ID NOs: 36 and 37, SEQ ID NOs: 38 and 39, SEQ ID NOs: 40 and 41, SEQ ID NOs: 42 and 43, SEQ ID NOs: 44 and 45, SEQ ID NOs: 46 and 47, SEQ ID NOs: 48 and 49, SEQ ID NOs: 50 and 51, SEQ ID NOs: 52 and 53, SEQ ID NOs: 54 and 55, SEQ ID NOs: 56 and 57, SEQ ID NOs: 58 and 59, SEQ ID NOs: 60 and 61, SEQ ID NOs: 62 and 63, SEQ ID NOs: 64 and 65, SEQ ID NOs: 66 and 67, SEQ ID NOs: 68 and 69, SEQ ID NOs: 70 and 71, SEQ ID NOs: 72 and 73, SEQ ID NOs: 74 and 75, SEQ ID NOs: 76 and 77, SEQ ID NOs: 78 and 79, SEQ ID NOs: 80 and 81, SEQ ID NOs: 82 and 83, SEQ ID NOs: 84 and 85, SEQ ID NOs: 86 and 87, SEQ ID NOs: 88 and 89, SEQ ID NOs: 90 and 91, SEQ ID NOs: 92 and 93, and SEQ ID NOs: 94 and 95—Each of these pairs define a different target region in the mitochondrial genome. In total, the target regions defined by these pairs cover the entire mitochondrial genomic DNA).

In some embodiments, the probe pairs in the probe subsets are designed such that the target regions in the heavy strand defined by the probe pairs in the first probe subset overlap with complementary target regions in the light strand defined by the probe pairs in the second probe subset. In some embodiments, a target region in the heavy strand defined by a probe pair from the first probe subset is followed by an overlapping target region in the light strand defined by a probe pair from the second probe subset (a “neighboring target region”). In some embodiments, the overlap between two neighboring target regions is at least about 30, 40, 50, 60, 70, 80, 90, 100, 110, 120, 130, 140, or 150 nucleotides long, but no more than about 300, 275, 250, 200 or 180 nucleotides. In some embodiments, the overlap between two neighboring target regions is between 30 and 150 nucleotides long. In some embodiments, the overlap between two neighboring target regions is between 50 and 120 nucleotides long. In some embodiments, the overlap between two neighboring target regions is between 80 and 100 nucleotides long.

In some embodiments, all the ligation probes in a probe subset comprise a common (same) nucleotide sequence for the first primer annealing sequence, all the extension probes in the same probe subset comprise a common nucleotide sequence for the second primer annealing sequence, and the nucleotide sequences of the first primer annealing sequence and the second primer annealing sequence are different.

In some embodiments, each ligation probe further comprises a molecular tag (aka. a “barcode”) sequence, wherein the molecular tag sequence has a different nucleotide sequence for each ligation probe (i.e., each molecular tag is unique). In some embodiments, each extension probe further comprises a molecular tag region, wherein the molecular tag sequence has a different nucleotide sequence for each extension probe. In some embodiments, each ligation probe further comprises a first molecular tag sequence and each extension probe further comprises a second molecular tag sequence, wherein the first molecular tag sequence has a different nucleotide sequence for each ligation probe, wherein the second molecular tag sequence has a different nucleotide sequence for each extension probe, and wherein the first molecular tag sequence and the second molecular tag sequence have different nucleotide sequences from any other molecular tag sequence in the probe set. In some embodiments, each molecular tag sequence is different from any other molecular tag sequence. In some embodiments, each molecular tag sequence is at least 5, 8, 10, 15 nucleotides or at least 20 nucleotides in length, but each molecular tag sequence is not more than 40, 35, 30, or 25 nucleotides in length. In some embodiments, each molecular tag sequence is between 10 and 25 nucleotides in length. In some embodiments, each molecular tag sequence is 10, 11, 12, 13, 14, 15, 16, 17, 18, 19, 20, 21, 22, 23, or 24 nucleotides in length.

### Methods for Sequencing a Mitochondrial Genomic DNA

Another aspect of the disclosure is directed to a method for sequencing a mitochondrial genomic DNA. In some embodiments the method comprises contacting a sample comprising a denatured mitochondrial genomic DNA with the probe set described above; performing an enzymatic gap filling reaction to connect the ligation probe and the extension probe in each pair of probes, thereby producing a ligation product; amplifying the ligation product; and sequencing the amplified products.

In some embodiments, the amplifying is achieved using a first primer that anneals to the first primer annealing sequence and a second primer that anneals to the complementary strand of the second primer annealing sequence. In some embodiments, the sequencing is performed using next-generation sequencing. As used herein “next-generation sequencing” refers to oligonucleotide sequencing technologies that have the capacity to sequence oligonucleotides at speeds above those possible with conventional sequencing methods (e.g., Sanger sequencing), due to performing and reading out thousands to millions of sequencing reactions in parallel. Non-limiting examples of next-generation sequencing methods/platforms include Massively Parallel Signature Sequencing (Lynx Therapeutics); 454 pyro-sequencing (454 Life Sciences/Roche Diagnostics); solid-phase, reversible dye-terminator sequencing (Solexa/Illumina): SOLiD technology (Applied Biosystems); Ion semiconductor sequencing (ION Torrent); DNA nanoball sequencing (Complete Genomics); and technologies available from Pacific Biosciences, Intelligen Bio-systems, Oxford Nanopore Technologies, and Helicos Biosciences. Next-generation sequencing technologies and the constraints and design parameters are well known in the art (see, e.g., Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; Zhang et al., “The impact of next-generation sequencing on genomics”, J Genet Genomics, 2011, 38(3):95-109; (Nyren, P. et al. Anal Biochem 208: 17175 (1993); Bentley, D. R., Curr Opin Genet Dev 16:545-52 (2006); Strausberg, R. L., et al., Drug Disc Today 13:569-77 (2008); U.S. Pat. Nos. 7,282,337; 7,279,563; 7,226,720; 7,220,549; 7,169,560; 6,818,395; 6,911,345; US Pub. Nos. 2006/0252077; 2007/0070349; and 20070070349; which are incorporated by reference herein in their entireties).

In some embodiments, the method is performed on a plurality of samples comprising mitochondrial genomic DNA from different subjects. In some embodiments, the method is performed in a multiplexed manner. In some embodiments, multiplexing comprises labeling each captured mitochondrial genomic DNA sample (target region) from each subject with at least one additional molecular tag (“barcode”) at the amplifying stage, wherein the additional molecular tag is different from any molecular tag of the ligation probes and extension probes. In some embodiments, the additional molecular tag sequence is at least 5, 8, 10, 15 nucleotides or at least 20 nucleotides in length, but not more than 40, 35, 30, or 25 nucleotides in length. In some embodiments, the additional molecular tag sequence is between 10 and 25 nucleotides in length. In some embodiments, the additional molecular tag sequence is 10, 11, 12, 13, 14, 15, 16, 17, 18, 19, 20, 21, 22, 23, or 24 nucleotides in length.

In some embodiments, the additional molecular tag is added during the amplification stage. In some embodiments, the additional molecular barcode has the same nucleotide sequence for all target regions captured from one subject, thereby identifying the target regions captured from the subject's mitochondrial genomic DNA.

In some embodiments, the additional molecular tag is added by one or both of the amplification primers (i.e., the amplification primer comprises the molecular tag sequence 3′ to the region that specifically hybridizes to the target sequence). In some embodiments, a unique molecular tag is assigned to a subject and represents the mitochondrial DNA from that specific subject. In some embodiments, samples that are labeled with subject-specific unique molecular tags are mixed together and sequenced as a pool. The sequencing results from a pool of mitochondrial chromosomal DNA can be differentiated by subject based on the molecular tags.

Methods for Determining Relative mtDNA Content

Another aspect of the disclosure is directed to a method for determining relative mtDNA content in a sample. As used herein, the phrase “relative mtDNA content” refers to the ratio of the amount of mitochondrial genomic DNA relative to the amount of cellular genomic DNA, and is a measure of the abundance of mitochondria per cell (i.e., the more mitochondria present in a cell, the higher the relative mtDNA content).

In some embodiments, the method comprises denaturing mitochondrial genomic DNA (mtDNA) and nuclear DNA(nDNA) in a sample; capturing a target region of the denatured mtDNA in the sample using any of the probe set described above; capturing a target region of the denatured nDNA using at least one nDNA-targeting probe pair, wherein each nDNA-targeting probe pair comprises an nDNA-targeting ligation probe and an nDNA-targeting extension probe; determining the amount of mtDNA and the amount of nDNA; and determining the ratio of the amount of mtDNA versus the amount of nDNA.

In some embodiments, the relative mtDNA content in a sample is determined by performing real-time quantitative Polymerase Chain Reaction (PCR) on captured mtDNA. In some embodiments, the mtDNA amount is normalized to the amount of a nuclear genomic DNA control in the sample.

In some embodiments, the relative mtDNA content in a sample is determined by sequencing a sample comprising a mitochondrial genomic DNA (mtDNA) and nuclear genomic DNA (nDNA) and determining the ratio of mtDNA sequencing read counts and nDNA sequencing read counts.

In some embodiments, mtDNA is sequenced using the probe set described herein, and the nDNA is sequenced using at least one nDNA-targeting probe pair.

As used herein, an “nDNA-targeting probe pair” refers to a pair of probes that specifically hybridize to a nuclear chromosome on the same strand, and do not hybridize to a mitochondrial chromosome region. In some embodiments, the nDNA-targeting probe pair comprises an n-DNA targeting ligation probe and an n-DNA targeting extension probe. In some embodiments, an nDNA-targeting probe pair comprises probes that specifically hybridize to a sequence in a nuclear (genomic, non-mitochondrial) DNA. In some embodiments, each probe pair within each nDNA-targeting probe subset comprises a ligation probe and an extension probe wherein the ligation probe of the nDNA-targeting probe pair has a different nucleic acid sequence than the extension probe of the same nDNA-targeting probe pair.

In some embodiments, the ligation probe and the extension probe of an nDNA-targeting probe pair specifically hybridize to sequences that are at least 200 nucleotides, but no more than 600 nucleotides, apart on the same strand of the nuclear genomic DNA. The sequence between the ligation probe and the extension probe of a probe pair is said to be “captured” or “defined” by the probe pair. The sequence between the ligation probe and the extension probe of a probe pair is also called the “target region” of the probe pair. In some embodiments, the nDNA-targeting probes in a probe pair capture or define a target region that is between 200-600 nucleotides, between 300-500 nucleotides, or between 399-449 nucleotides in length. In some embodiments, each probe pair captures (or “defines”) a target region that is about 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 nucleotides long.

In some embodiments, each nDNA-targeting ligation probe comprises a first primer annealing sequence and a 5′-phosphorylated ligation arm that is substantially complementary to a sequence at a 5′ border (a first end) of a target region on the nuclear genomic DNA defined by the probe pair. In some embodiments, the ligation arm comprises between 15 and 45 nucleotides. In some embodiments, the ligation arm is about 15, 18, 20, 25, 28, 30, 35, 38, 40, or 45 nucleotides long. In some embodiments, the ligation arm is at least 15, 18, 20, 25, 28, 30, or 35 nucleotides long, but is no longer than 80, 70, 60, or 50 nucleotides.

In some embodiments, each nDNA-targeting extension probe comprises a second primer annealing sequence and an extension arm that is substantially complementary to a sequence at a 3′ border (a second end) of the target region on the nuclear genomic DNA defined by the probe pair. In some embodiments, the extension arm comprises between 15 and 45 nucleotides. In some embodiments, the extension arm is about 15, 18, 20, 25, 28, 30, 35, 38, 40, or 45 nucleotides long. In some embodiments, the extension arm is at least 15, 18, 20, 25, 28, 30, or 35 nucleotides long, but is no longer than 80, 70, 60, or 50 nucleotides.

In some embodiments, the ligation arm of an nDNA-targeting probe does not anneal to an identical or overlapping sequence on the genomic DNA with the extension arm of an nDNA-targeting probe. In some embodiments at least 3, at least 5, at least 8, or at least 10 nDNA-targeting probe pairs are used in the method for determining relative mt-DNA content.

In a specific embodiment, the ligation arms and extension arm sequences of nDNA-targeting probe pairs are selected from pairs shown in Table 4, by SEQ ID NOs: 96 and 97, SEQ ID NOs: 98 and 99, SEQ ID NOs: 100 and 101, SEQ ID NOs: 102 and 103, and SEQ ID NOs: 104 and 105.

In some embodiments, an nDNA-targeting probe pair comprises a ligation probe and an extension probe, and both the ligation probe and the extension probe anneal to the same strand of a nuclear genomic DNA. In some embodiments, each nDNA probe pair defines a target region of the nuclear genomic DNA that is not identical to any other target region defined by any other nDNA probe pair. In some embodiments, each nDNA-targeting probe is designed against a single copy target region of the nuclear DNA.

In some embodiments, all the ligation probes (including all nDNA and mtDNA ligation probes) comprise a common nucleotide sequence for the first primer annealing sequence, all the extension probes (including all nDNA and mtDNA extension probes) comprise a common nucleotide sequence for the second primer annealing sequence, and the nucleotide sequences of the first primer annealing sequence and the second primer annealing sequence are different. In some embodiments, each nDNA ligation probe further comprises a molecular tag sequence, wherein the molecular tag sequence is unique for each ligation probe (including all nDNA and mtDNA ligation probes). In some embodiments, each nDNA extension probe further comprises a molecular tag region, wherein the molecular tag sequence is unique for each extension probe (including all nDNA and mtDNA extension probes). In some embodiments, each ligation probe further comprises a first molecular tag sequence and each extension probe further comprises a second molecular tag sequence, wherein the first molecular tag sequence is unique for each ligation probe (including all nDNA and mtDNA ligation probes), wherein the second molecular tag sequence is unique for each extension probe (including all nDNA and mtDNA extension probes), and wherein the first molecular tag sequence and the second molecular tag sequence are different from each other. In some embodiments, each molecular tag sequence is different from any other molecular tag sequence. In some embodiments, each molecular tag sequence is at least 5, 8, 10, 15 nucleotides or at least 20 nucleotides in length, but each molecular tag sequence is not more than 40, 35, 30, or 25 nucleotides in length. In some embodiments, each molecular tag sequence is between 10 and 25 nucleotides in length. In some embodiments, each molecular tag sequence is 10, 11, 12, 13, 14, 15, 16, 17, 18, 19, 20, 21, 22, 23, or 24 nucleotides in length.

### Methods for Detecting Mutations in a Mitochondrial Genomic DNA

Yet another aspect of the disclosure is directed to detecting mutations in a mitochondrial genomic DNA.

In some embodiments, the method comprises sequencing a mitochondrial DNA as described above, and further processing the sequencing data to determine whether any mutation exists in the mitochondrial genomic DNA.

In some embodiments, the further sequencing comprises removing from sequencing reads sequences of the primer annealing regions, thereby producing trimmed reads, aligning the trimmed reads based on the molecular tag regions, wherein aligned reads with identical molecular tag regions represent PCR duplicates from one probe pair and aligned reads with different molecular tag regions represent an overlapping region from different probe pairs; and determining whether a mutation exists in the aligned trimmed reads. When a mutation is detected in the aligned reads, the mutation is classified as a true variant when the mutation is found in all members of aligned reads with identical molecular tag regions, and the mutation is classified as an error (e.g., a PCR error (a mutation introduced during the PCR amplification) or a sequencing error (a mutation introduced during sequencing, a misreading of the base)) when the mutation is not found in all members of aligned reads with identical molecular tag regions.

### Methods for Designing a Probe Set

An aspect of the disclosure is directed to a method of designing a probe set for sequencing a mitochondrial genomic DNA. In some embodiments, the method comprises designing a probe set that comprises a first probe subset comprising a plurality of probe pairs and a second probe subset comprising a plurality of probe pairs. As used in this disclosure, the phrase “plurality of probe pairs” refers to at least 5, least 10, at least 12, at least 15, at least 20, at least 25, or at least 30 probe pairs in each probe subset. In a specific embodiment, the phrase “plurality of probe pairs” refers to 23, 24 or 25 probe pairs in each probe subset.

In some embodiments, each probe pair within each probe subset comprises a ligation probe and an extension probe wherein the ligation probe of the probe pair has a different nucleic acid sequence than the extension probe of the same probe pair. In some embodiments, each probe pair in the first probe subset comprises probes (i.e., ligation probe and extension probe pairs) that specifically hybridize to sequences in the heavy strand of a mitochondrial genomic DNA, and each probe pair in the second probe subset comprises probes (i.e., ligation probe and extension probe pairs) that specifically hybridize to sequences in the light strand of a mitochondrial genomic DNA. In some embodiments, the ligation probe and the extension probe of a probe pair specifically hybridize to sequences that are at least 200 nucleotides, but no more than 600 nucleotides, apart on the same strand of the mitochondrial genomic DNA. The sequence between the ligation probe and the extension probe of a probe pair is said to be “captured” or “defined” by the probe pair. The sequence between the ligation probe and the extension probe of a probe pair is also called the “target region” of the probe pair. In some embodiments, the probes in a probe pair capture or define a target region that is between 200-600 nucleotides, between 300-500 nucleotides, or between 399-449 nucleotides in length. In some embodiments, each probe pair captures (or “defines”) a target region that is about 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 nucleotides long.

In some embodiments, each ligation probe comprises a first primer annealing sequence and a 5′-phosphorylated ligation arm that is substantially complementary to a sequence at a 5′ border (a first end) of a target region on the mitochondrial genomic DNA defined by the probe pair. In some embodiments, the ligation arm comprises between 15 and 45 nucleotides. In some embodiments, the ligation arm is about 15, 18, 20, 25, 28, 30, 35, 38, 40, or 45 nucleotides long. In some embodiments, the ligation arm is at least 15, 18, 20, 25, 28, 30, or 35 nucleotides long, but is no longer than 80, 70, 60, or 50 nucleotides.

In some embodiments, each extension probe comprises a second primer annealing sequence and an extension arm that is substantially complementary to a sequence at a 3′ border (a second end) of the target region on the mitochondrial genomic DNA defined by the probe pair. In some embodiments, the extension arm comprises between 15 and 45 nucleotides. In some embodiments, the extension arm is about 15, 18, 20, 25, 28, 30, 35, 38, 40, or 45 nucleotides long. In some embodiments, the extension arm is at least 15, 18, 20, 25, 28, 30, or 35 nucleotides long, but is no longer than 80, 70, 60, or 50 nucleotides.

In a specific embodiment, the target region is about 300-500 nucleotides long (i.e., the ligation probe and the extension probe of a probe pair specifically hybridize to sequences that are about 300-500 nucleotides apart), and the ligation arm of the ligation probe that specifically hybridizes to the 5′ border (first end) of the target region is between 15-35 nucleotides long and the extension arm of the extension probe that specifically hybridizes to the 3′ border (second end) of the target region is between 15-35 nucleotides long.

In some embodiments, each ligation and extension probe pair define a target region of the mitochondrial genomic DNA that is not identical to any other target region defined by any other probe pair. In some embodiments, the target regions defined by the first probe subset and the target regions defined by the second probe subset in combination cover the entirety of the mitochondrial genomic DNA.

In some embodiments, the ligation arm does not anneal (specifically hybridize) to an identical or overlapping sequence on the mitochondrial genomic DNA with the extension arm.

In a specific embodiment, the ligation arm of the ligation probe and the extension arm of the extension probe of a probe pair are selected from the pairs recited in Table 4 (i.e., selected from the mt-DNA-specific pairs shown by SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, SEQ ID NOs: 34 and 35, SEQ ID NOs: 36 and 37, SEQ ID NOs: 38 and 39, SEQ ID NOs: 40 and 41, SEQ ID NOs: 42 and 43, SEQ ID NOs: 44 and 45, SEQ ID NOs: 46 and 47, SEQ ID NOs: 48 and 49, SEQ ID NOs: 50 and 51, SEQ ID NOs: 52 and 53, SEQ ID NOs: 54 and 55, SEQ ID NOs: 56 and 57, SEQ ID NOs: 58 and 59, SEQ ID NOs: 60 and 61, SEQ ID NOs: 62 and 63, SEQ ID NOs: 64 and 65, SEQ ID NOs: 66 and 67, SEQ ID NOs: 68 and 69, SEQ ID NOs: 70 and 71, SEQ ID NOs: 72 and 73, SEQ ID NOs: 74 and 75, SEQ ID NOs: 76 and 77, SEQ ID NOs: 78 and 79, SEQ ID NOs: 80 and 81, SEQ ID NOs: 82 and 83, SEQ ID NOs: 84 and 85, SEQ ID NOs: 86 and 87, SEQ ID NOs: 88 and 89, SEQ ID NOs: 90 and 91, SEQ ID NOs: 92 and 93, and SEQ ID NOs: 94 and 95—Each of these pairs define a different target region in the mitochondrial genome. In total, the target regions defined by these pairs cover the entire mitochondrial genomic DNA).

In some embodiments, the probe pairs in the probe subsets are designed such that the target regions in the heavy strand defined by the probe pairs in the first probe subset overlap with complementary target regions in the light strand defined by the probe pairs in the second probe subset. In some embodiments, a target region in the heavy strand defined by a probe pair from the first probe subset is followed by an overlapping target region in the light strand defined by a probe pair from the second probe subset (a “neighboring target region”). In some embodiments, the overlap between two neighboring target regions is at least about 30, 40, 50, 60, 70, 80, 90, 100, 110, 120, 130, 140, or 150 nucleotides long, but no more than about 300, 275, 250, 200 or 180 nucleotides. In some embodiments, the overlap between two neighboring target regions is between 30 and 150 nucleotides long. In some embodiments, the overlap between two neighboring target regions is between 50 and 120 nucleotides long. In some embodiments, the overlap between two neighboring target regions is between 80 and 100 nucleotides long.

In some embodiments, all the ligation probes in a probe set comprise a common nucleotide sequence for the first primer annealing sequence, all the extension probes in the same probe set comprise a common nucleotide sequence for the second primer annealing sequence, and the nucleotide sequences of the first primer annealing sequence and the second primer annealing sequence are different.

In some embodiments, each ligation probe further comprises a molecular tag (aka. a “barcode”) sequence, wherein the molecular tag sequence has a different nucleotide sequence for each ligation probe (i.e., each molecular tag is unique). In some embodiments, each extension probe further comprises a molecular tag region, wherein the molecular tag sequence has a different nucleotide sequence for each extension probe. In some embodiments, each ligation probe further comprises a first molecular tag sequence and each extension probe further comprises a second molecular tag sequence, wherein the first molecular tag sequence has a different nucleotide sequence for each ligation probe, wherein the second molecular tag sequence has a different nucleotide sequence for each extension probe, and wherein the first molecular tag sequence and the second molecular tag sequence have different nucleotide sequences from any other molecular tag sequence in the probe set. In some embodiments, each molecular tag sequence is different from any other molecular tag sequence. In some embodiments, each molecular tag sequence is at least 5, 8, 10, 15 nucleotides or at least 20 nucleotides in length, but each molecular tag sequence is not more than 40, 35, 30, or 25 nucleotides in length. In some embodiments, each molecular tag sequence is between 10 and 25 nucleotides in length. In some embodiments, each molecular tag sequence is 10, 11, 12, 13, 14, 15, 16, 17, 18, 19, 20, 21, 22, 23, or 24 nucleotides in length.

### Methods of Determining Mitochondrial Mutation Load or Degree of Heteroplasmy in a Subject

Another aspect of the disclosure is directed to a method for determining mitochondrial mutation load or degree of heteroplasmy in a subject.

“Mitochondrial mutation load” refers to the totality of mutations accumulated in a subject's mitochondrial genomic DNA. Increased mitochondrial mutation load can lead to mitochondrial diseases (including, but not limited to, MELAS (Mitochondrial encephalopathy, lactic acidosis, and stroke-like episodes Syndrome), NARP (Neuropathy, ataxia, and retinitis pigmentosa), Leigh's Syndrome, MERRF (myoclonic epilepsy with ragged red fibers) Syndrome, Leber's hereditary optic neuropathy (LHON), Kern-Sayre Syndrome, Mitochondrial neurogastrointestinal encephalopathy syndrome (MNGIE), Aplers Disease) or exacerbate diseases where mitochondrial biology plays a role (including, but not limited to, Huntington's Disease, Alzheimer Disease and cancer).

In some embodiments, the subject is suffering from a disease and determining the mitochondrial mutation load in the subject can facilitate an understanding of the underlying cause or severity, or determining the subtype of the disease. In some embodiments, the mutational load is predictive of, or indicative of, disease severity and prognosis. In some embodiments, the subject is suffering from a mitochondrial disease selected from the group consisting of MELAS (Mitochondrial encephalopathy, lactic acidosis, and stroke-like episodes Syndrome), NARP (Neuropathy, ataxia, and retinitis pigmentosa), Leigh's Syndrome, MERRF (myoclonic epilepsy with ragged red fibers) Syndrome, Leber's hereditary optic neuropathy (LHON), Kern-Sayre Syndrome, Mitochondrial neurogastrointestinal encephalopathy syndrome (MNGIE), and Aplers Disease. In a specific embodiment, the subject is suffering from Huntington's Disease, Alzheimer's Disease or cancer.

In some embodiments, the subject is suspected to be suffering from a disease and determining the mitochondrial mutation load in the subject can predict the onset or severity of the disease. In a specific instance, the instant methods can be used to diagnose a mitochondrial disease.

In some embodiments, the method comprises contacting a sample comprising a denatured mitochondrial genomic DNA with the probe set described above; performing an enzymatic gap filling reaction to connect the ligation probe and the extension probe in each pair of probes, thereby producing a ligation product; amplifying the ligation product; and sequencing the amplified products.

In some embodiments, the amplifying is achieved using a first primer that anneals to the first primer annealing sequence and a second primer that anneals to the complementary strand of the second primer annealing sequence. In some embodiments, the sequencing is performed using next-generation sequencing.

In some embodiments, the method is performed on a plurality of samples comprising mitochondrial genomic DNA from different subjects. In some embodiments, the method is performed in a multiplexed manner. In some embodiments, multiplexing comprises labeling each captured mitochondrial genomic DNA sample (target region) from each subject with at least one additional molecular tag (“barcode”) at the amplifying stage, wherein the additional molecular tag is different from any molecular tag of the ligation probes and extension probes. In some embodiments, the additional molecular tag sequence is at least 5, 8, 10, 15 nucleotides or at least 20 nucleotides in length, but not more than 40, 35, 30, or 25 nucleotides in length. In some embodiments, the additional molecular tag sequence is between 10 and 25 nucleotides in length. In some embodiments, the additional molecular tag sequence is 10, 11, 12, 13, 14, 15, 16, 17, 18, 19, 20, 21, 22, 23, or 24 nucleotides in length.

In some embodiments, the additional molecular tag is added during the amplification stage. In some embodiments, the additional molecular barcode has the same nucleotide sequence for all target regions captured from one subject, thereby identifying the target regions captured from the subject's mitochondrial genomic DNA.

In some embodiments, the additional molecular tag is added by one or both of the amplification primers (i.e., the amplification primer comprises the molecular tag sequence 3′ to the region that specifically hybridizes to the target sequence). In some embodiments, a unique molecular tag is assigned to a subject and represents the mitochondrial DNA from that specific subject. In some embodiments, samples that are labeled with subject-specific unique molecular tags are mixed together and sequenced as a pool. The sequencing results from a pool of mitochondrial chromosomal DNA can be differentiated by subject based on the molecular tags.

In some embodiments, once a mutation is detected by sequencing, it is classified as a real mutation or an artifact (an error). In some embodiments, a mutation is classified as a true variant when the mutation is found in all members of aligned reads with identical molecular tag regions (a molecular tag region identifies a specific region of the mitochondrial genome, thus, all reads that have the same molecular tag (barcode) is a sequence of the same region), and classifying the mutation as an error when the mutation is not found in all members of aligned reads with identical molecular tag regions. In some embodiments, the error is a sequencing error (misreading of a base), or a PCR artifact (a wrong base introduced due to DNA duplication error during the amplification stage).

Another aspect of the disclosure is directed to a method of determining heteroplasmy (“mithochondrial heteroplasmy”) in a subject. The term “heteroplasmy” or “mtDNA heteroplasmy” refers to mtDNA mutations that arise and co-exist with the wild-type allele in the same cell. The phrase “degree of heteroplasmy” refers to the amount of heteroplasmy in a given cell. As there are multiple copies of mtDNA in a given cell, low degree of heteroplasmy (e.g., less than 50% of mutant mtDNA) may not show any phenotypes. However, increasing degree of heteroplasmy (e.g., above 60%, 70%, 80%, 90%, 95%, 99%, of mtDNA in a cell is mutated compared to wild type, rendering the mitochondria dysfunctional) may result in a disease state, or may exacerbate an already-existing condition.

In some embodiments, the method comprises contacting a sample comprising a denatured mitochondrial genomic DNA with a probe set as described herein; performing an enzymatic gap filling reaction to connect the ligation probe and the extension probe in each pair of probes, thereby producing a ligation product; amplifying the ligation product; sequencing the amplified products; removing from sequencing reads sequences of the primer annealing regions, thereby producing trimmed reads; aligning the trimmed reads based on the molecular tag regions, wherein aligned reads with identical molecular tag regions represent PCR duplicates from one probe pair and aligned reads with different molecular tag regions represent an overlapping region from different probe pairs; determining whether heteroplasmy exists in the aligned trimmed reads, wherein when a mutation is detected, classifying the mutation as a heteroplasmy variant when the mutation is found in an overlapping region from different probe pairs; and thereby determining the heteroplasmy in a subject.

In some embodiments, the sequencing is performed using next-generation sequencing.

In some embodiments, the probe pairs in the probe subsets are designed such that neighboring target regions in the heavy strand defined by the probe pairs in the first probe subset overlap with neighboring complementary target regions in the light strand defined by the probe pairs in the second probe subset.

In some embodiments, a target region in the heavy strand defined by a probe pair from the first probe subset is followed by an overlapping target region in the light strand defined by a probe pair from the second probe subset.

In some embodiments, heteroplasmy is detected when a mutation is consistently detected in both the heavy chain and light mitochondrial genomic DNA (mtDNA). A mutation is considered “consistently detected” when the same mutation is observed/detected from overlapping neighboring target sites that are on different chains of mtDNA. In some embodiments, heteroplasmy is calculated by the ratio of mutation-containing subset and wild-type subset of mtDNA. In some embodiments, the amount of mutation-containing subset and wild-type subset of mtDNA is measured by sequencing read counts.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

To sequence whole mitochondrial genome, 46 pairs of probes were designed to capture mtDNA from human total genomic DNA. Each pair of probe consists of a ligation probe and an extension probe. The ligation probe has 5′-phosphorylated ligation arm complementary to the DNA target sequence and 20-nt common primer annealing region at 3′ terminus. The extension probe has a 15-nt unique molecular tag flanked by 3′ target-specific extension arm and another 20-nt constant PCR primer annealing region. The ligation and extension arms were designed such that they would hybridize immediately upstream and downstream of capturing targets cover regions ranged from 399 to 449 mer long in mtDNA. Adjacent pairs of probes were designed to target on heavy and light strand of mtDNA alternatively. After hybridization of probes with mtDNA targets, an enzymatic gap-filling and ligation reaction were used to seal the gap between the probes. A pair of PCR primers appended with sample-specific barcode and Illumina adapters which directed at the common PCR primer annealing regions was used to amplify the capture product. To alleviate the problems of amplification bias and artifacts, the molecular tag consisting of 15 random nucleotides were used to track independent capture events. Sequence reads that have different molecular tags represent different original captured target molecules, while reads that have the same tags are highly likely PCR duplicates arise from the same captured target. For a family of duplicates share the same molecular tag, random polymerase errors may be present in only one or a few members of the family. These artifacts represent sequencing mistakes or PCR introduced errors occurring late in amplification. They can be distinguished from true variants which appear in all members of a family By removing the PCR artifacts from the true variants, the duplicated reads are consolidated into a single representative read and use to calculate the relative abundance of target in the original capture product. This method accomplishes mitochondrial DNA selection, library preparation and molecular tagging in a simple workflow. It has robust repeatability and reproducibility; it enables highly sensitive detection of low-frequency variation, it also allows rapid and high-throughput analysis of mitochondrial DNA in large scale.

### Study Samples

Two HapMap lymphoblast cell lines (sample 1: NA12751, and sample 2: NA18523) were purchased from Coriell Institute. Upon receiving them, the lymphoblast cell lines were revived and cultured, at 37° C. with 5% CO₂, in RPMI 1640 medium containing 15% fetal bovine serum (VWR Life Science Seradigm, Inc.) and 1× Antibiotic-Antimycotic (Thermo Fisher Scientific, Inc.). Total genomic DNA of these two samples was obtained using Wizard Genomic DNA Purification Kit (Promega, Inc.) as per the manufacturer's instructions. The concentration of purified DNA was quantified by using a Qubit dsDNA HS assay kit (Thermo Fisher Scientific, Inc.). The five DNA sample mixtures were created by combining total genomic DNA of these two HapMap samples at relative ratios of 1:199, 1:99, 5:95, 20:80, and 50:50 (NA12751 versus NA18523). The lymphoblast cell line samples from 200 healthy control individuals were collected in REGISTRY (PLoS Curr., 2, RRN1184) and DNA were extracted as per REGISTRY protocol.

mtDNA Sequencing with STAMP

The experimental and computational protocols of STAMP used in the current study for sequencing mtDNA are listed as follows.

Step 1. mtDNA Capture with Extension-Ligation (EL) Probes

The oligos of EL probe pairs for each of the 46 mtDNA target regions and 5 nDNA target regions were column-synthesized, at 25 nanomole scale with standard desalting purification (Integrated DNA Technologies, Inc.). In order to improve uniformity of sequencing coverage on mtDNA, aliquots of the 51 EL probe pairs were pooled. Hybridization reactions were performed on 50 ng genomic DNA with 4 ul EL probe mix and 1× Ampligase buffer in a 10 μl volume. Thermal conditions included 10 min at 95° C. for denaturation, followed by a decrease of 1° C. per min to 55° C. and 20 h at 55° C. for hybridization. 6 μl gap-filling mix including 0.1 mM dNTPs, 0.6M Betaine, 0.1M (NH₄)₂SO₄, 0.5 units of Tsp DNA polymerase and 0.5 units of Ampligase in 1× Ampligase buffer was then added to the reaction mixture, which was incubated at 55° C. for another 20 h for gap filling.

### Step 2. PCR Amplification of Capture Products

The inventors used a dual indexing strategy to pool sample libraries for parallel sequencing. Each indexing primer comprised P5 or P7 Illumina adapter sequences, an 8-nt index sequence, a 13- or 14-nt pad sequence, and a universal sequence designed at the 3′ terminus of extension or ligation probe. (27) PCR amplification was performed on 1.5 μl of capture product in a 50 μl PCR reaction with 1× Phusion HF buffer, 0.5 μM of p5i5 and p7i7 indexing primers, 0.2 mM dNTP, and 1 unit of Phusion Hot-Start II DNA polymerase (Thermo Scientific, Inc.). PCR thermal conditions were 30 sec at 98° C. for initial denaturation, followed by 25 cycles of 10 sec at 98° C., 15 sec at 65° C., and 15 sec at 72° C. The size and integrity of PCR products were visually verified by agarose gel electrophoresis.

### Step 3. Library Purification

The obtained PCR products were purified and filtered by using Ampure XP magnetic beads with double size selection (Beckman Coulter, Inc.). In brief, 0.25 volume of beads was first used to bind DNA of >700 bp in the PCR products, after which the supernatant was transferred to a fresh tube. An extra 0.4 volume of beads was added to bind DNA of >500 bp in the supernatant. After the beads were washed and dried, DNA bound to these beads which contained PCR products of size in the range of 550 bp to 650 bp were eluted with 10 mM Tris-HCl, pH8.5, and were quantified with QUBIT® 2.0 Fluorometer (Life Technologies, Inc.). Equal amounts of purified PCR products from different samples were pooled and used as libraries for parallel sequencing.

### Step 4. Massively Parallel Sequencing

The sample libraries were sequenced with customized sequencing primers and 2×250 paired-end reads on Illumina sequencing flow cells. The Read 1 primer contained the 13-nt pad sequence and the 20-nt universal sequence (TGCACGTCATCTACAGTAGGTCGGTGCGTAGGT) (SEQ ID NO: 1) of the ligation probe. The Read 2 primer contained the 14-nt pad sequence and the 20-nt universal sequence (CTCACTGGAGTTCAAGGGACGATGAGTGGCGATG) (SEQ ID NO: 2) of the extension probe. The Index primer was the reverse complement of the Read 2 primer sequence (CATCGCCACTCATCGTCCCTTGAACTCCAGTGAG) (SEQ ID NO: 3), which along with the complementary adapter sequences on the flow cell was used to read the dual sample indices. Cluster generation, image processing, and sequencing for samples of the current study were processed on MiSeq or HiSeq 2500 in the rapid run mode. Phi-X DNA library was spiked in at 5% to increase the complexity of the STAMP sequencing libraries.

### Step 5. Sequencing Data Processing

A Python pipeline was developed to process and align paired-end reads generated from STAMP. In brief, paired-end reads were first demultiplexed into files of individual samples based on the i5 and i7 index sequences. For each individual sample, paired-end reads were sorted into 51 clusters of capture products according to the arm region sequences identified at the locations of EL probes. The arm region sequences and the molecular barcode were trimmed from the paired-end reads, which were recorded in the read alignment files as annotations. To minimize complications from NUMTS in mtDNA read alignment, paired-end reads were first aligned to the reference human genome containing both nuclear DNA (genome assembly GRCh 38) and mtDNA (Revised Cambridge Reference Sequence, rCRS) sequences downloaded from bwa mem, version 0.7.17. Paired-end reads, annotated as having one of the 46 mtDNA EL probes, were marked as potential NUMTs in the alignment file if they could also be aligned to nuclear DNA with MAPQ≥10. Paired-end reads were aligned in a second round to a modified version of rCRS which had the final 120 bp copied to the start to accommodate alignment of D-loop-region reads with the D10 probes. Paired-end reads that could not be aligned to the target region specified by their arm region sequences were removed. The remaining reads were locally realigned by using freebayes (version 1.1.0) and their base qualities were subsequently recalibrated by using samtools (version 1.6).

For paired-end reads with the same molecular barcode, the base information called at corresponding sites of the alignments was merged by using a Bayesian approach to generate a consensus read representing the captured mtDNA product. The same method was also used to merge base information within the overlapping region of the paired-end reads. The sequences of consensus reads were compared to a collection of known NUMTS sequences in the reference genome obtained from BLASTN search of the 46 mtDNA segments captured with EL probes, as well as their variant sequences harboring common polymorphisms (minor allele fraction >1%) identified in the 1000 Genomes project. A consensus read was marked as potential NUMTS if it showed a lower pairwise edit distance to NUMTS sequences than to the sample's major mtDNA sequence, or if it was constructed from paired-end reads already annotated as NUMTs according to BWA alignment. Finally, consensus reads were converted to single-end reads, along with their base quality information, and stored in a bam file for each individual sample.

mtDNA Variant Detection

mtDNA variants were determined by using consensus reads with MAPQ≥20 and BAQ≥30. Consensus reads marked as NUMTS or showing an excess of mismatches (>5 in the coding region and >8 in the D-loop region; >11 for sample mixtures) compared to the individual's major mtDNA sequence were also excluded from analysis. To reduce false positive calls of heteroplasmies, variants were subject to a list of quality filters, including (1) ≥100× depth of coverage with ≥70% of the bases having BAQ≥30; (2) not in low-complexity regions (nt 302-316, nt 512-526, nt 16814-16193) or low-quality sites (nt 545, 16224, 16244, 16249, 16255, and 16263); (3) ≥5 minor alleles detected; (3) a log likelihood quality score of the variant ≥5; (4) comparable VAFs (Fisher's exact test P≥10⁴ and fold change ≤5) computed using consensus reads constructed with or without duplicate paired-end reads; (5) the detected number of minor alleles significantly larger than the expected number of errors, which was estimated at a rate of 0.02% in STAMP (Exact Poisson test, P<0.01/16569).

Functionalities for raw read processing, paired-end read alignment, consensus read calling, and variant detection have been implemented in the STAMP tool kit.

mtDNA Content Evaluation with Quantitative PCR

The relative mtDNA content of 126 lymphoblast samples, with enough genomic DNA after STAMP sequencing, was measured by using a quantitative PCR-based assay. The PCR reactions were performed as per manufacturer's instructions (The Detroit R&D, Inc.). In brief, 15 ng total genomic DNA was amplified with mtDNA or nDNA target primers and SYBR green PCR master mix in a 20 μL PCR reaction. Thermal conditions included 10 min at 95° C., followed by 40 cycles of 15 sec at 95° C. and 60 sec at 60° C. For each sample, both mtDNA and nDNA targets were amplified twice in a total of 4 PCRs. Results from duplicates were averaged to compute mean Ct values for mtDNA and nDNA targets. The differences between them (ΔC_{T}) were then normalized to that of a positive control sample measured on the same 96-well plate, by using ΔΔC_{T} method, to obtain qPCR-CN. qPCR-CN from 10 samples that failed in any of the 4 PCRs, and/or had a difference in C_{T} values of over 3 cycles between experimental duplicates, were excluded from analysis.

### Study Subjects

Two HapMap lymphoblast cell lines (sample 1: NA12751, and sample 2: NA18523) were purchased from Coriell Institute. Upon receiving them, the lymphoblast cell lines were revived and cultured, at 37° C. with 5% CO₂, in RPMI 1640 medium containing 15% fetal bovine serum (VWR Life Science Seradigm, Inc.) and 1× Antibiotic-Antimycotic (Thermo Fisher Scientific, Inc.). Total genomic DNA of these two samples was obtained using Wizard Genomic DNA Purification Kit (Promega, Inc.) as per the manufacturer's instructions. The concentration of purified DNA was quantified by using a Qubit dsDNA HS assay kit (Thermo Fisher Scientific, Inc.). The five DNA sample mixtures were created by combining total genomic DNA of these two HapMap samples at relative ratios of 1:199, 1:99, 5:95, 20:80, and 50:50 (NA12751 versus NA18523). The lymphoblast cell line samples from 200 healthy individuals were collected in REGISTRY, a multi-center, prospective observational study of HD in Europe (Orth, M. et al., PLoS Curr. 2, RRN1184 (2011)).

### Example 2: Design of STAMP

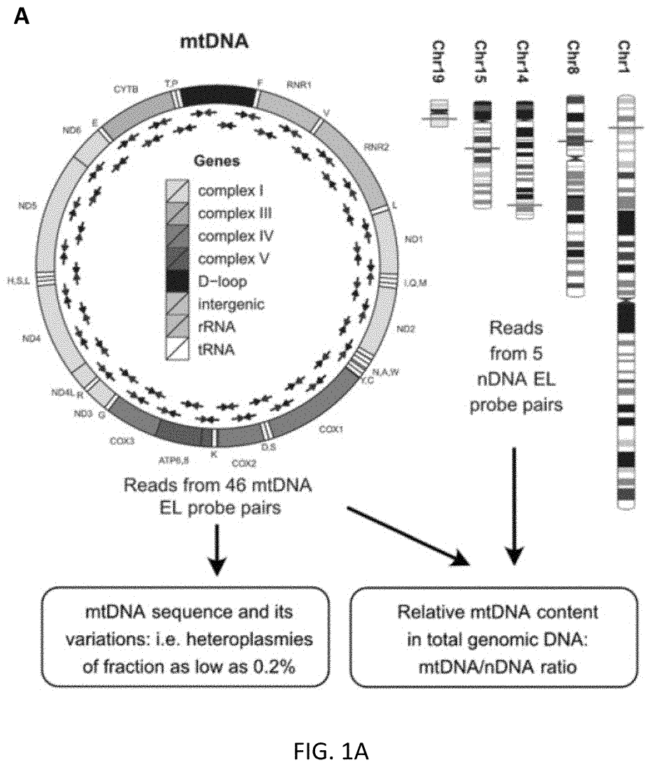

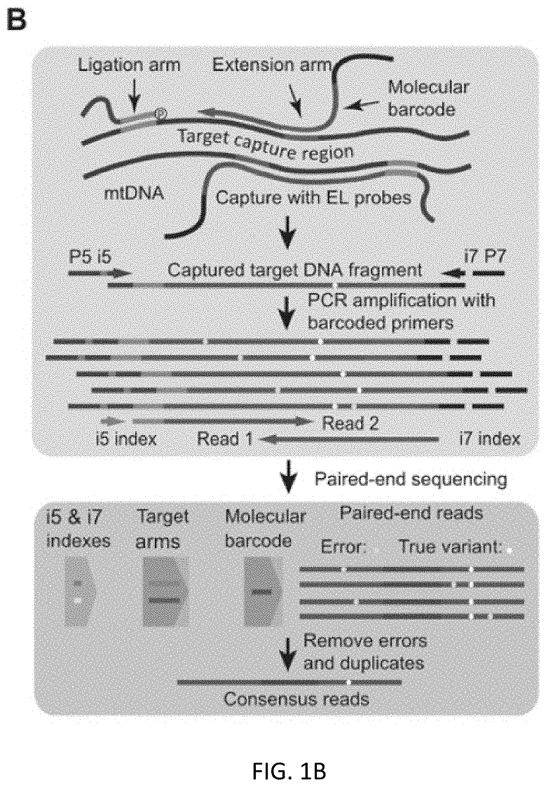

The inventors designed single-stranded oligonucleotide probes to capture human mtDNA with an extension-ligation (EL) reaction (FIG. 1A). The extension probe has three parts, a 3′ extension arm with sequence complementary to the mtDNA target, a 12-nt unique molecular tag used for tracking the capturing event, and a 20-nt common PCR primer annealing region used for PCR amplification of the captured target. The ligation probe has a 5′-phosphorylated ligation arm with sequence complementary to the mtDNA target, along with another 20-nt common PCR primer annealing region at its 3′ end (FIG. 1A).

To identify the required number of EL probe pairs and their mtDNA target locations, the inventors first performed BLASTN search of human mtDNA sequences against the latest human reference genome (assembly GRCh38). The inventors required that the resulting mtDNA segments be distinguishable from high-similarity segments derived from the human nuclear genome. Given the maximum sequencing read length of available Illumina sequencing platforms, the inventors also required that the lengths of mtDNA targets should be around 400 bp, so that they could be fully sequenced by using 2×250 or 2×300 paired-end reads while the overlapping between the paired end reads are minimal.

As a result, the inventors found that the entire 16.6 kb human mitochondrial genome could be captured by using as low as 46 pairs of EL probes. The inventors then placed the pairs of EL probes on the heavy and light strands of mtDNA alternatingly, to minimize the physical interference of adjacent probes in the multiplex reaction (FIG. 1A). The locations and lengths of the extension and ligation arms in each of the EL probe pairs were further adjusted to ensure similar melting temperatures, around 55° C., and similar GC-content, around 50%, and to avoid overlap with common mtDNA polymorphisms (population frequency >1%) at the 3′ ends of the extension and ligation arms. In the end, the inventors obtained 46 pairs of EL probes with a mtDNA target size ranging from 400 to 450 bp to capture human mtDNA.

### Example 3: Effective Capture of mtDNA with EL Probes

In a pilot experiment, the inventors synthesized the set of 46 pairs of EL probes (Integrated DNA Technologies, Inc.). The inventors performed enzymatic gap-filling and ligation reactions on 50 ng genomic DNA extracted from a lymphoblast cell line sample from the HapMap project, with 115 femtomoles of EL probe mixture. The PCR amplification of the captured targets, using the 20-nt common PCR primers, requires the presence of PCR primer annealing regions at both ends due to successful polymerization of nucleic acids between the hybridized EL probes, as well as ligation of the polymerized nucleic acids with the 5′ end of the ligation arm. Therefore, captured products which lacked one of the common primer sequences, due to failed hybridization of either probe with its target sequences, or no ligation at the 5′ end of the ligation arm, could not be amplified.

Without adding genomic DNA templates or necessary enzymes, no clear bands were observed in the gel electrophoresis of PCR products from the gap-filling reactions. In contrast, gel electrophoresis of PCR products from the effective gap-filling reaction on genomic DNA exhibited a smear, which had a size distribution centered at about 550 bp-600 bp, reflecting the expected sizes of PCR products comprising the target DNA, the molecular barcode, the probe arms, the common primers, and the sequencing adapters.

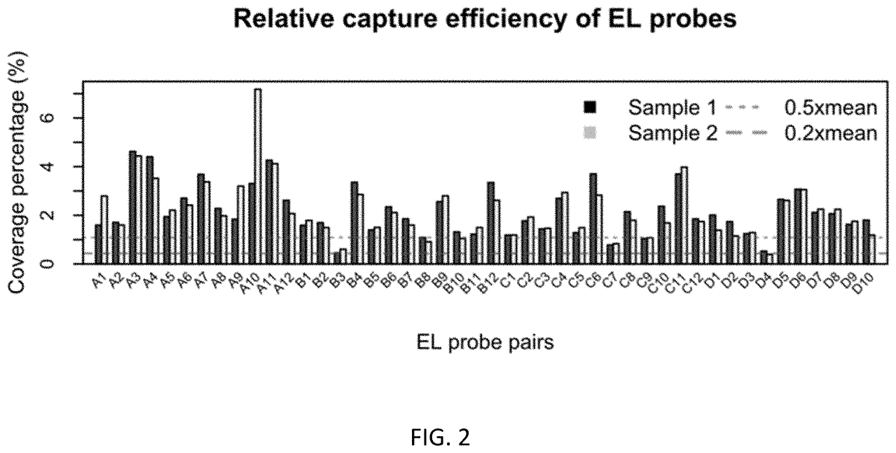

Next, the inventors purified PCR products with Ampure XP magnetic beads (Beckman Coulter, Inc.) and sequenced them with 2×250 bp paired-end reads on MiSeq (Illumina, Inc.). After mapping the paired-end reads to the reference human genome, the inventors found that all of the 46 mtDNA target regions were covered with reads at an average sequencing depth of 3512×, confirming that the set of 46 pairs of EL probes was able to capture the full length of human mtDNA (FIG. 2). The inventors further replicated these results in a second lymphoblast sample from the HapMap project (FIG. 2). There was also a high correlation of coverage of regions captured by the same EL probe pair between these two samples (Pearson's r=0.8, P=3.3×10⁻¹¹).

Moreover, capturing mtDNA sequences from genomic DNA could be potentially biased by the presence of nuclear DNA regions with high sequence similarity to mtDNA (i.e. nuclear mitochondrial segments, NUMTS). To reveal its influence on mtDNA capture in STAMP, the inventors performed gap-filling and ligation reactions with the set of 46 EL probes on genomic DNA from 143B.TK⁻ mtDNA-less (ρ0) cell line, and compared its PCR products with those obtained from the parental cell line 143B.TK⁻ which contains mtDNA. The gel electrophoresis of corresponding PCR products showed that the characteristic smear was only detectable in 143B.TK⁻ cells, but was absent in 143B.TK⁻ρ0 cells, indicating that the resulting PCR products were largely derived from captured mtDNA target regions rather than off-target NUMTs.

### Example 4: Utilization of Molecular Barcodes to Identify PCR Duplicates and Improve Sequencing Accuracy

Most mtDNA heteroplasmies are at low factions at the tissue level, which require a high depth of coverage of reads to reveal the presence of the variant allele and assess their fraction in relation to the wide-type allele. However, an ultra-deep read coverage (i.e., >2000×) on the small 16.6 Kb human mtDNA may create errors in removing read duplicates if they are solely determined based on read coordinates and sequences. PCR amplification of mtDNA before sequencing may also introduce biases in estimating VAF of a heteroplasmy. To resolve these issues, in STAMP, a 12- or 15-random-nucleotide molecular barcode was incorporated via the EL probe pairs to each of the capture products before PCR amplification, creating an identity for each capturing event. Therefore, paired-end reads from the same mtDNA fragment captured in STAMP, including duplicates, can be determined according to the attached barcode information (FIG. 1B).

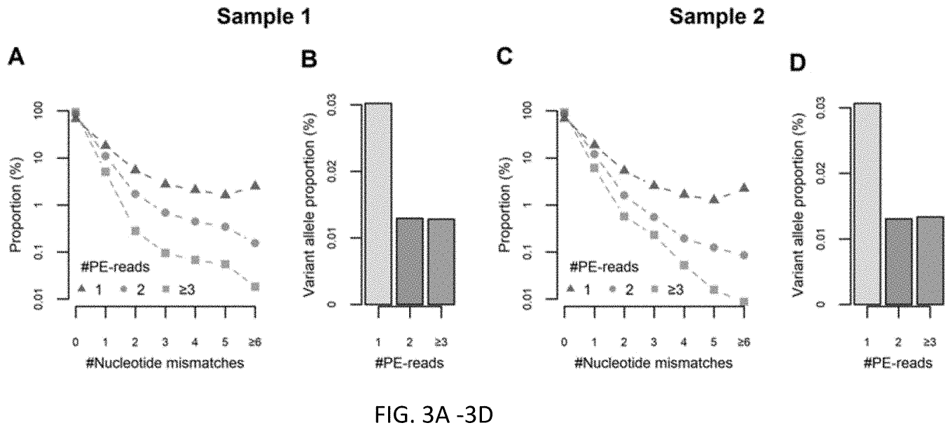

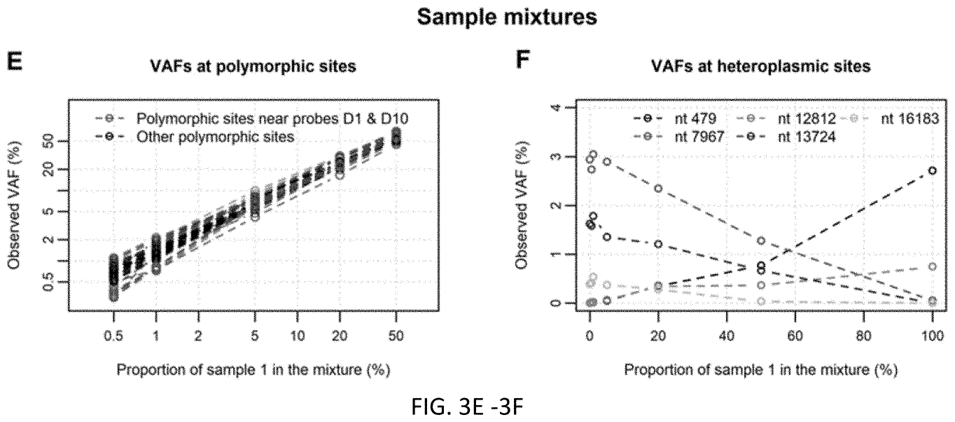

Moreover, nucleotide mismatches at corresponding sites of paired-end reads with the same molecular barcode would suggest either PCR artifacts or sequencing errors (FIG. 1B). The inventors employed a Bayesian approach to merge the base information of these paired-end reads, generating a consensus read representing the captured DNA fragment. The inventors found that the number of nucleotide mismatches between the sequences of the consensus read and the reference mtDNA significantly decreased after merging base information of paired-end reads (Kolmogorov-Smirnov test, P<2.2×10⁻¹⁶, FIG. 3A). Consensus reads with an excess of mismatches (NM>5) in comparison to the reference mtDNA were almost undetectable if they were constructed with duplicate paired-end reads, with a frequency 30-fold less than those of consensus reads without duplicate paired-end reads (Chi-squared test, P<2.2×10⁻¹⁶).

After filtering out consensus reads with an excess of mismatches (NM>5) and bases with low quality scores (BAQ<30), the inventors found that the proportion of variant alleles, which encompassed PCR and sequencing errors as well as low-level heteroplasmies, was about 0.013% and 0.03% per base among consensus reads with and without duplicate paired-end reads, respectively (FIG. 3B). Both values are considerably lower than the reported proportions of ˜0.1% per base in commonly-used mtDNA-targeted sequencing methods. The proportion of variant alleles, at 0.013% per base, is also close to the error rate of Tsp DNA polymerase used in the gap-filling reaction of STAMP, which can be further improved using DNA polymerase with a higher fidelity. The inventors obtained similar reductions in the distribution of nucleotide mismatches, and variant allele proportions, in the consensus reads of the second sample (FIGS. 3C and 3D).

### Example 5: Accurate Detection of mtDNA Heteroplasmies and their Fractions in Total Genomic DNA

To evaluate the sensitivity and specificity of STAMP for detecting mtDNA heteroplasmies and their fractions in total genomic DNA, the inventors applied STAMP to a series of sample mixtures created by combining total genomic DNA from the two lymphoblast samples used in the pilot experiment at varying ratios, ranging from 1:199 to 1:1. mtDNA sequences of these two lymphoblast samples differ at 59 single nucleotide sites. One site (nt 16189) was in a low-complexity poly-C region of mtDNA and was excluded from the analysis of heteroplasmies. The average mean depth of coverage of consensus reads on mtDNA among the 5 sample mixtures was 3938× (median depth: 3284×), comparable to that of the two original samples at 3988× (median depth: 3392×).

The inventors found that all 58 polymorphic sites exhibited changes in their VAFs in accordance with the ratios of DNA used to generate these sample mixtures (r>0.997, P<0.00017; FIG. 3E). Of note, 2 polymorphic sites were located within the annealing regions of the EL probes: nt16519 at the 2^{nd} position of the extension arm of probe D10, and nt13650 at the 10^{th} position of the extension arm of probe D1. These two polymorphisms did not abolish the hybridization of EL probes to the target regions (for reasons discussed in the next section), but the variance in the VAFs of the 18 heteroplasmies close to the annealing regions of probes D1 and D10 was increased compared to that of the other heteroplasmies, especially in low-fraction sample mixtures (2-sided F-test P<1.4×10⁻⁵, FIG. 3E). This suggests that a nucleotide mismatch between the arm regions of EL probes and their annealing regions in mtDNA can alter DNA capture efficiency, affecting the estimation of VAFs of nearby heteroplasmies in linkage with the nucleotide mismatch.

However, the pilot experiment using the sample mixtures represents an extreme scenario where all the 58 polymorphisms are in complete linkage with each other, and their alleles are separable into two haplogroups of mtDNA. In real human samples, the incidence of medium- and high-fraction heteroplasmies is usually low, and new heteroplasmies tend to arise in different mitochondria. Therefore, the variant and wild-type alleles of a heteroplasmy tend to share the same flanking mtDNA sequences. Both alleles would have the same rate of capture by EL probe pairs in the same reaction.

Next, the inventors examined the influence of applying rigid quality control filters on detecting low-fraction mtDNA variants in the 3 sample mixtures, created with genomic DNA ratios of 1:199, 1:99 and 5:95. The inventors found that 5 out of the 174 (3×58) variants were unable to survive the quality filtering procedures described in Example 1. Of these, three showed a low percentage of high-quality reads and two were located at sites that did not have a number of sufficient reads containing the variant alleles. Moreover, all the 174 variants had VAF greater than 50% of the ratios of the genomic DNA in the sample mixtures. Therefore, STAMP's sensitivity of identifying low-fraction heteroplasmies (VAF=0.5%-5%) is over 97%, with a cutoff for VAF at 0.25% and an average coverage of consensus reads at about 4000×.

By using these quality control filters, the inventors found 17 other mtDNA heteroplasmies at VAF≥0.25%. All of them were located at the 5 heteroplasmic sites already detected in one of the two original mtDNA samples, at a VAF from 0.4% to 2.9%. These 5 heteroplasmic sites also displayed VAF changes proportional to the ratios of the DNA in the sample mixtures (r>0.9, P<0.0046; FIG. 3F). Therefore, the false positive rate of STAMP in detecting heteroplasmies of VAF≥0.25% is under 10⁻⁴ (1/16569) per site of mtDNA.

### Example 6: Application of STAMP in a Population Study of mtDNA Heteroplasmies

To demonstrate the effectiveness and robustness of using STAMP for assessing mtDNA heteroplasmies in larger numbers of samples, the inventors used STAMP to sequence mtDNA in 200 lymphoblast samples collected in REGISTRY. These 200 lymphoblast samples constituted the healthy control group of the research project on Huntington's disease, and were sequenced on HiSeq2500 along with other samples relating to this project.

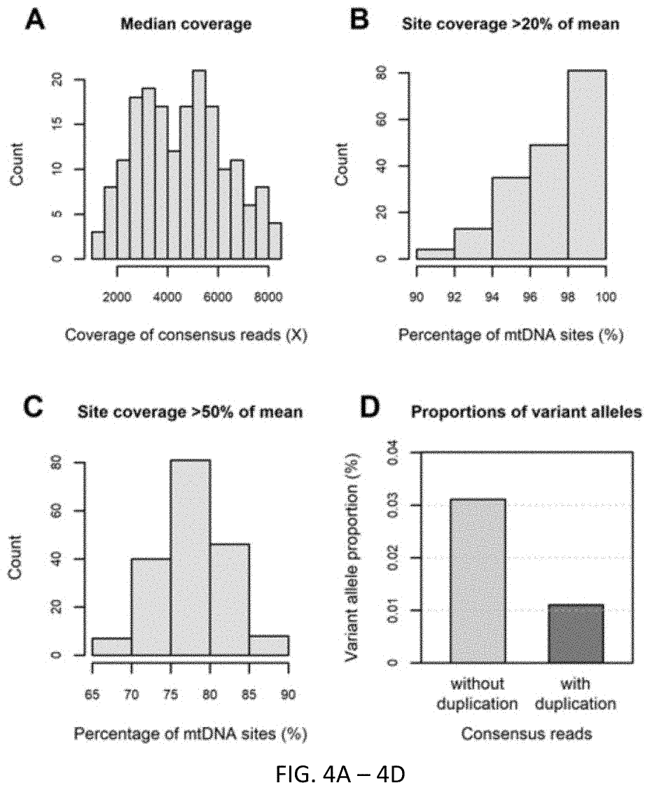

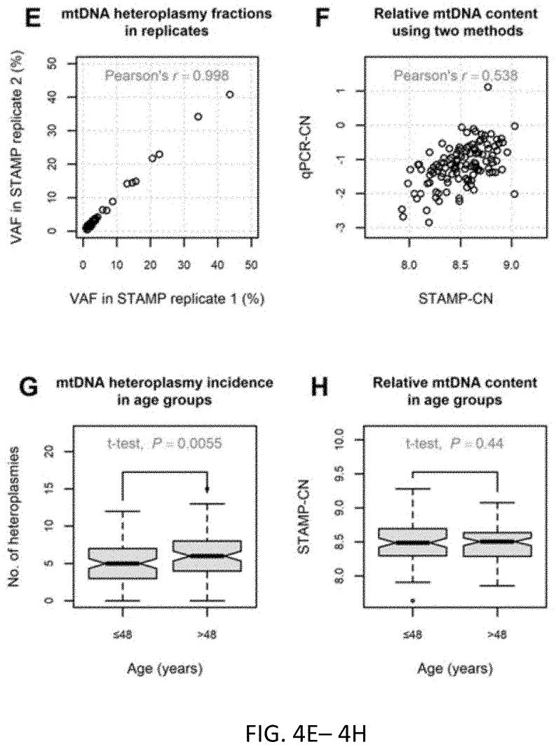

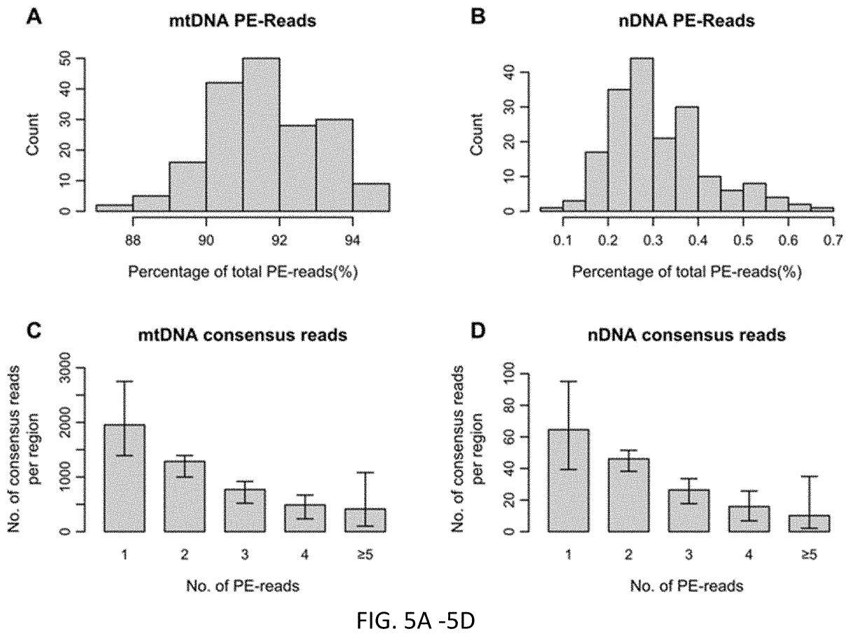

The inventors were able to build sequencing libraries for 192 (92%) out of 208 samples, including the experimental replicates from 8 lymphoblast samples. Among the 192 samples with STAMP libraries, 190 (99%) libraries from 182 lymphoblast samples were sequenced to >1000× depth of median coverage of consensus reads on mtDNA. The average median and mean depths of coverage of consensus reads on mtDNA were 4580× and 5450×, respectively (FIG. 4A). The percentages of mtDNA sites that were covered with over 20% and 50% of the mean depth of coverage of consensus reads, as indicators for read coverage uniformity, were 98% and 78%, respectively (FIGS. 4B and 4C). 99.4% mtDNA sites were covered with >500 consensus reads and 96.3% were covered with >1000 consensus reads. Similar to the observations from the two lymphoblast samples of the HapMap project, the overall proportions of variant alleles were 0.011% and 0.031% per base of consensus reads constructed with and without duplicate paired-end reads, respectively (FIG. 4D). Taken together, these results confirm that STAMP can effectively sequence mtDNA in larger-scale studies.

Of note, two low-frequency mtDNA polymorphisms (nt2626 and nt15758) were identified at the 3′ end of EL probes A6 and D7 in two samples. These two mtDNA polymorphisms are single base transitions from A to G or T to C which give rise to purine-pyrimidine (A-C, C-A, G-T, and T-G) mismatches between the mtDNA templates and the arm regions of the EL probes. Since purine-pyrimidine mismatches in the 3′ end regions of primers have less detrimental impacts on PCR amplification than purine-purine or pyrimidine-pyrimidine mismatches, these two probes still produced 1248 and 268 consensus reads in the corresponding samples. These reads accounted for 0.3% and 0.4% of total consensus reads obtained, or equivalently 17% and 25% of the average proportions of consensus reads captured by EL probes A6 and D7 from mtDNA, respectively.

Since >90% of known polymorphisms, as well as heteroplasmies, in mtDNA cause single base transitions and are outside the 3′ end regions of EL probes, the current design of EL probes can be applied to capturing the entire length of most mtDNA haplogroups worldwide. However, any population other than Europeans, that were investigated in the current study, may lead to different coverage of consensus reads on mtDNA if there are ethnicity-specific common polymorphisms or substitutions located in the arm regions of EL probes, especially close to the 3′ ends. Therefore, a pilot experiment of STAMP, aiming to identify appropriate molar ratios of EL probes that can ensure read uniformity on mtDNA for a population of interest, may be needed before large-scale applications. The inventors further list all possible common polymorphisms (population frequency >1%) located in the arm regions of EL probes to help improve the design of EL probe sequences for non-European populations.

Among the 8 lymphoblast samples with replicated STAMP measures, the inventors found that the major mtDNA sequences were identical between sequencing replicates. All the 45 heteroplasmies identified with VAF≥1% in one sample had VAF≥0.5% in the replicate, 44 (98%) of which also passed all quality control filters for heteroplasmy calling. Overall, the correlation between VAFs of the heteroplasmies detected in sequencing replicates was estimated to be r=0.998 (P=3×10⁻⁵³, FIG. 4E). The average coefficient of variation in repeated measures of VAFs, at median site coverage of 4330×, was 5% for medium-to-high-fraction heteroplasmies (VAF≥5%; median VAF=15%) and was 17% for low-fraction heteroplasmies (0.5%≤VAF<5%; median VAF=1.4%). These values are close to the corresponding estimates of 4% and 13% computed using the sampling distribution of sample proportions at the VAF medians. These results indicate that STAMP can reliably detect heteroplasmies and quantify their fractions in genomic DNA.

### Example 7: Extension of STAMP to Measure mtDNA Content in the Same Assay

The inventors further explored the possibility of modifying STAMP to enable mtDNA content quantification in the same assay. To this end, the inventors added five pairs of EL probes to capture single-copy regions in nuclear DNA (nDNA), along with the 46 pairs of mtDNA EL probes in STAMP. These five target nDNA regions are located on different autosomal chromosomes (FIG. 1A). Reads from the nDNA regions can be used as a normalization factor to adjust differences in total genomic DNA input and sequencing coverage across samples.

The inventors first evaluated the performance of the nDNA EL probe pairs in capturing their target regions relative to the mtDNA EL probe pairs. The inventors have noted in the previous analyses that the presence of polymorphisms in the arm regions of the EL probes could influence the capture efficiency of the target region. The inventors thus focused on a subset of the 46 EL probe pairs to compute an average number of consensus reads for mtDNA. This subset comprised 18 EL probe pairs (A5-A8, B2, B6, B7, B9, C1-05, C7, C9, C12, D1, D5) that lack common polymorphisms in their arm regions in European populations, and showed relatively low variations in consensus read coverage across samples of the current study.

The inventors found that all the five nDNA probe pairs exhibited positive correlations in their consensus read numbers with that of mtDNA EL probe pairs (R²=0.4-0.79). To improve reliability in estimating nDNA content in the sample, the inventors used the 3 EL probe pairs targeting chromosomes 8, 4, and 19 with an R²>0.74 to compute an average number of consensus reads for nDNA. In addition, the inventors found that the performance of EL probe pairs was not equal when capturing nDNA compared to mtDNA target regions, possibly due to a compact design of EL probes on mtDNA. To adjust for this difference, the inventors computed the relative mtDNA content for STAMP (hereafter referred to as STAMP-CN) as the average consensus read number from mtDNA relative to that from nDNA, by using the equation: log₂(No. of mtDNA consensus reads)—C×log₂(No. of nDNA consensus reads). C in the equation stands for the normalization factor for nDNA consensus reads, estimated using the coefficient β from the regression of log₂(No. of mtDNA consensus reads) against log₂(No. of nDNA consensus reads), which was equal to 0.53.

By comparing STAMP-CN with the relative mtDNA content (hereafter referred to as qPCR-CN) was determined using a commercially available quantitative PCR-based assay performed on the same sample, the inventors found a significant positive correlation (r=0.54, P=10⁻⁵, FIG. 4F) between the values of STAMP-CN and qPCR-CN. This correlation is in good agreement with the result reported in a previous comparative study of mtDNA content measured by sequencing-based methods and qPCR-based methods (Tsang C. J. H. et al., Genome Biol., 16, 1-16). It indicates that STAMP-CN can reflect the relative mtDNA content in total genomic DNA, comparable to those from qPCR-based methods designed for mtDNA content assessment.

### Example 8: Age-Dependent Increase of Heteroplasmy Incidence in Lymphoblast Samples

With both mtDNA heteroplasmies and mtDNA content measured in the same sample, the inventors then evaluated how age impacts these mtDNA characteristics in lymphoblast samples as compared to what the inventors previously observed in mtDNA of blood samples, using the WGS dataset of the UK10K project.

The inventors identified 1007 heteroplasmies of VAF≥1% across the entire length of mtDNA in 182 lymphoblast samples of REGISTRY (FIG. 5A). The average number of heteroplasmies per sample was 5.5 (range:0-15). 180 (99%) lymphoblasts possessed at least one heteroplasmy in mtDNA (FIG. 5B). Similar to the inventors' previous study on lymphoblast mtDNA, the number of mtDNA heteroplasmies identified in lymphoblasts was consistently greater than those of whole blood, at about 1 heteroplasmy of VAF≥1-2%, implying that mtDNA of lymphoblasts may enrich for pre-existing variants in somatic cells that are undetectable at a tissue level, or new mutations created during the establishment of the cell lines.

At the variant level, 760 (88%) of the 862 identified heteroplasmic sites were unique to one of the 182 lymphoblast samples (FIG. 5C). Over half (54%) of the heteroplasmic sites did not overlap with known mtDNA polymorphisms (a population frequency <0.01%), and another 20% were found to overlap only with rare polymorphisms in less than 0.1% of the general population (FIG. 5D). The base changes of heteroplasmies showed a high transition to transversion ratio at 15. This suggests that the dominant mutational force underlying heteroplasmies is nucleotide misincorporation by polymerase gamma or deamination of bases in mtDNA, consistent with mtDNA mutation patterns identified in blood.

The inventors first performed Student's t-test to compare mtDNA heteroplasmies and content between individuals aged above and under the sample median of 48 years old. The inventors found increased mtDNA heteroplasmy incidence and decreased mtDNA content in lymphoblast samples in the older group (mean age: 55 years old) compared to those in the younger group (mean age: 41 years old). But only mtDNA heteroplasmy incidence showed a significant difference between these two age groups (FIG. 4G; Cohen's d=0.42, P=0.0055). The lack of a significant association of mtDNA content with age (FIG. 4H; Cohen's d=−0.12, P=0.44 for STAMP-CN; Cohen's d=−0.07, P=0.71 for qPCR-CN) might be due to insufficient statistical power, since only a mild annual decrease of 0.4 mtDNA copies (0.24% of the population average in 1511 individuals, P=0.0097) in blood was noted in the inventors' previous study (Zhang R. et al., BMC Genomics, 18, 890).

Furthermore, by using a Poisson regression model in the analyses of heteroplasmy incidence, the inventors found an annual rate of increase of 1.2% (95% CI: 0.5%-2.0%; P=0.00063) for heteroplasmies of VAF≥1% in lymphoblast samples (Table 1, model 1). In line with the inventors' previous findings in blood ((Zhang R. et al., BMC Genomics, 18, 890), the increase of heteroplasmy incidence in lymphoblast samples with age was not affected by changes in mtDNA content (Table 1, model 2). Moreover, the inventors found a similar age-dependent increase of heteroplasmy incidence after focusing on unique variants detected in the dataset, meaning that random genetic events in mtDNA, such as replication errors or drift, are largely responsible for the accumulation of mtDNA heteroplasmies during aging (Table 1, model 3). Significant age effects were also obtained using heteroplasmies of higher VAFs (VAF≥2% or VAF≥5%, Table 1).

These results indicate that the age-dependent accumulation of mtDNA heteroplasmies may be conserved in lymphoblast samples, after immortalization of B lymphocytes by Epstein-Barr virus and short cultivation of the cell lines. Therefore, lymphoblast samples may serve as a useful genetic resource for studying age-related mtDNA mutation spectra in the hematopoietic system, and their contributions to mitochondrial dysfunction in diseases associated with aging.

### Example 9

An aspect of the instant disclosure presents a novel human mtDNA targeted sequencing method, STAMP, which enables assessment of mtDNA sequence variations and mtDNA content at a low cost. This method streamlines the experimental workflow with multiplex capture of human mtDNA and nDNA, and generates high-quality sequencer-ready libraries in one tube. This novel methodology eliminates the error-prone steps of transferring reagents and DNA samples, reduces the risk of DNA contamination, and enables mtDNA sequencing in thousands of samples.

Importantly, with high cost-effectiveness and a flexible design, STAMP can be used to study mtDNA variations at different scales and to determine mtDNA heteroplasmies at different fraction levels. Given the 0.01%-0.03% error rates of STAMP, STAMP can be used to detect heteroplasmies of fractions as low as <0.5%, with deeper sequencing coverage. Thus, STAMP can be used in studies of somatic mtDNA mutations in tissue specimens, which is currently unachievable by using other mtDNA-targeted sequencing methods, or whose experimental cost is prohibitive, when a large number of samples need to be sequenced.

Moreover, the inventors provide in the current disclosure the related experimental details and computational solutions to assist the application of STAMP in future human mtDNA studies. Accordingly, the insights gained from these studies will transform the inventors' understanding of the role of mtDNA in aging and age-related diseases of humans.

TABLE 1
Age-dependent increase of mtDNA heteroplasmy incidence in lymphoblast samples.

| | Model 1 | | Model 2 | | Model 3 | |
|---|---|---|---|---|---|---|
| mtDNA | Beta | | Beta | | Beta | |
| heteroplasmy | [95% CI] | P | [95% CI] | P | [95% CI] | P |
| VAF ≥ 1% | 0.012 | 0.00063 | 0.012 | 0.00073 | 0.014 | 0.0012 |
| | [0.005-0.020] | | [0.005-0.019] | | [0.005-0.022] | |
| VAF ≥ 2% | 0.017 | 0.00087 | 0.017 | 0.00091 | 0.020 | 0.00043 |
| | [0.007-0.027] | | [0.007-0.026] | | [0.009-0.031] | |
| VAF ≥ 5% | 0.027 | 0.00025 | 0.027 | 0.00026 | 0.037 | 2.7 × 10⁻⁵ |
| | [0.013-0.042] | | [0.013-0.042] | | [0.020-0.054] | |

The associations between heteroplasmy incidence and age were computed by using Poisson log-linear model with adjustment for sex and sequencing coverage in model 1, and for sex, sequencing coverage, and the relative mtDNA content (STAMP-CN) in model 2 and model 3. In model 3, only heteroplasmies that occurred once in the 182 lymphoblast samples were used to compute the incidence.

### Example 10: Cost-Effectiveness and Flexibility of STAMP Applications

The integration of mtDNA capture and enrichment with multiplex probes in STAMP can effectively reduce the cost of sequencing library construction to under S5. When the inventors call mtDNA variants, the minimum VAF of the heteroplasmies and the statistical power to distinguish them from sequencing and PCR errors are both affected by read depths and error rates of sequencing. Both parameters can be adjusted in STAMP by changing the numbers of consensus reads and paired-end reads, allowing the sequencing costs and scales to be flexible according to the aim of the study.

The number of consensus reads obtained for mtDNA in STAMP reflects the number of mtDNA fragments (NF) captured with EL probes. By fitting a Poisson distribution to the numbers of paired-end reads used in constructing consensus reads in the 182 lymphoblast samples of the current study, the inventors found that NF was close to 6000 per EL probe target region in 1.5 ul of capture product from STAMP sequencing performed on 50 ng of lymphoblast DNA. Yet, NF may vary, depending on the extraction methods, tissue sources and quality of the genomic DNA. Therefore, the inventors recommend conducting a pilot experiment with STAMP on 10-20 samples in one lane of MiSeq to estimate NF empirically. The obtained NF can then be used to calculate the amount of capture product that needs to be amplified and sequenced, to ensure enough consensus reads for detecting mtDNA heteroplasmies.

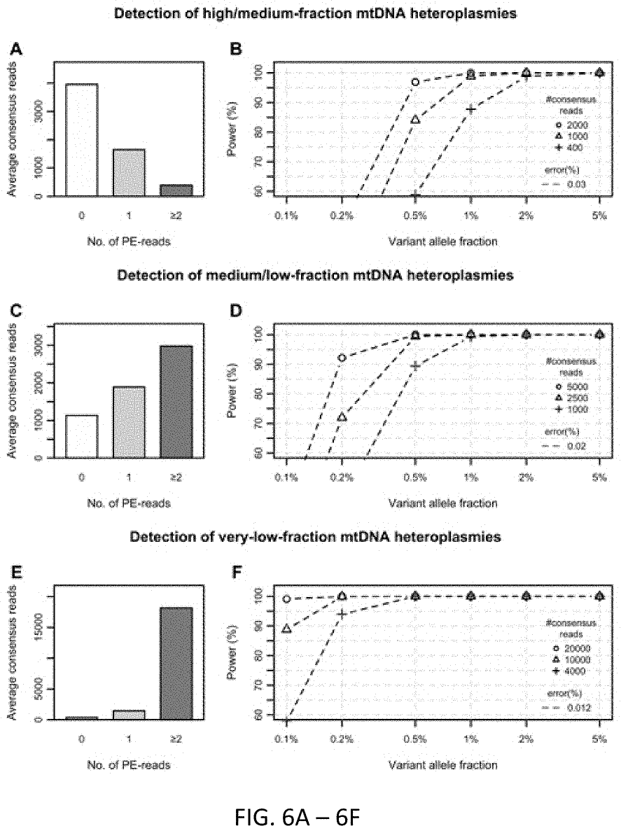

For example, in the current study, 1.5 ul of capture product contained roughly an average of 6000 unique mtDNA fragment for each of the 46 EL probes. Among the 190 lymphoblast samples, the rate of paired-end reads retained for constructing consensus reads for mtDNA and nDNA was 0.9 and 0.003, respectively, after alignment and quality filtering. Given a yield of 125 million 2×250 bp paired-end reads from one lane of a flow cell processed on HiSeq 2500, a batch load of 250 sample libraries on each lane can produce an average of about 10000 (0.9×125×10⁶/46/250) and 300 (0.003×110×10⁶/5/250) paired-end reads per EL probe region in mtDNA and nDNA, respectively, for each sample. Accordingly, each consensus read will be constructed from an average of about 2 paired-end reads. About 60% of consensus reads will have duplication, which improves the error rate of STAMP from 0.03% to 0.02% per base (FIG. 6B). At this error rate, STAMP guarantees >99% power to distinguish heteroplasmies of VAFs at 1% and 0.5% from errors, at an average of 98% and 78% of mtDNA sites, respectively (FIG. 6C).

Similarly, very-low-fraction heteroplasmies can be detected by further increasing the numbers of consensus reads and paired-end reads. For example, 20,000 consensus reads per EL probe region and 80,000 paired-end reads can be achieved by amplifying 5 ul of capture products, and sequencing the resulting libraries in a batch load of 31 samples on one lane of HiSeq 2500. As a result, >92.5% consensus reads will incorporate information from at least 2 paired-end reads, and, on average, 4 paired-end reads which lowers the error rate to 0.012% per base and provides >99% and >94% power for detecting heteroplasmies at VAF of 0.2% for 78% and 98% of mtDNA sites, respectively (FIGS. 6E and 6F).

However, if the aim of the study is to assess medium- or high-fraction heteroplasmies, polymorphisms, or haplogroups, which do not require ultra-deep read depths to detect, increasing the number of either consensus reads or paired-end reads may waste sequencing capacity. Under these circumstances, a batch load of up to 1000 sample libraries per lane on HiSeq 2500 can be applied to achieving an average coverage of consensus reads and paired-end reads at 2000× and 2500×, which can be used to detect heteroplasmies of VAF≥2% (FIGS. 6A and 6B). Moreover, according to the observed ratio of nDNA and mtDNA reads in the current study (FIGS. 5A-5B), an average of over 50 consensus reads can still be attained from the nDNA regions for computing STAMP-CN.

### Example 11: Implementation of the STAMP Tool Kit

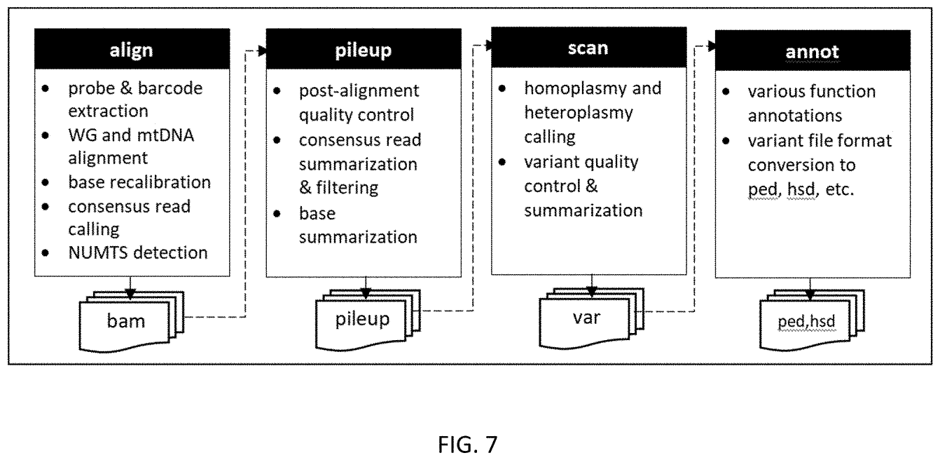

The inventors developed a Python pipeline (the STAMP tool kit) to process sequencing data. Each functionality described in the main text has been implemented in the STAMP tool kit (hereafter referred to as “Stamp”) and is summarized in the flow chart below. Stamp has four modules, “align”, “pileup”, “scan”, and “annot”, as shown in FIG. 7.

Below is the command to list all the stamp modules:

python stamp.py-h

Usage: stamp.py<command>[options]

Command: align generate the consensus read alignments
pileup summarize the consensus read bases
scan variant identification
annot variant annotation

### Example 12: Read Alignment and Consensus Read Calling

In the “align” module, stamp reads the raw fastq files, and extracts the probe arm and molecular barcode sequences from the paired-end reads according to the design of EL probes in STAMP (FIG. 1B). The sequences of the probe arms must be from one of the 46 mtDNA and 5 nDNA probe pairs with a maximum mismatch of 3 bases in either the extension arm or the ligation arm. Because of sequencing errors, a maximum of 3 nucleotide mismatches is allowed between the arm sequence and the matched probe sequence. The molecular barcode must contain at least 9 bases with BAQ≥15. The paired-end reads that pass these quality filters are exported into individual fastq files with the barcode and probe information retained in the read description.

The paired-end reads are then aligned to the complete reference genome containing both nuclear DNA (genome assembly GRCh38) and mtDNA (Revised Cambridge Reference Sequence, rCRS) sequences using “bwa mem”:

bwa mem -L 100, 5-M genome_reference.fa filtered_R1.fastq filtered_R2.fastq “-L 100,5” disables soft clips following the trimmed probe arm sequences in the paired-end reads. These reads are then aligned again to a revised rCRS with the final 120 bp copied to the start to accommodate alignment of reads in the D-loop region:

bwa mem -L 100, 5-M shifted_mtdna.fa filtered_R1.fastq filtered_R2.fastq: The paired-end reads that are unmapped, not in proper pairs, or not aligned to the correct chromosome or location as per the design of EL probe targets (MAPQ<20), are excluded. The paired-end reads from the 46 mtDNA EL probe pairs are marked as “NUMTS” in the alignment file if they are mapped to nDNA in the complete reference genome (MAPQ≥10).

The properly aligned paired-end reads are locally realigned with freebayes(2) and the base qualities are recalibrated with samtools.

bamleftalign -c -f|samtools calmd-Earb: Based on the attached molecular barcode, the recalibrated paired-end reads are grouped into read families. The sequence of the consensus read is determined for each read family using a Bayesian approach. In brief, the posterior probability of having a nucleotide, such as “A”, at a certain position in the consensus read can be represented using the equation below,

$$P ( A \vert all reads ) = \frac{{\prod }_{i = 1}^{n} P ( {read}_{i} \vert A ) \times P ( A )}{{\sum }_{NT} {\prod }_{i = 1}^{n} P ( {read}_{i} \vert NT ) \times P ( NT )}$$

where P(NT) is prior probability and Πᵢ₌₁ⁿP(readᵢ|NT) is the estimated likelihood, under the assumption that all paired-end reads in a read family are independent. To simplify calculation, the inventors use equal prior probability for all nucleotides. The likelihood of a nucleotide in each read can be approximated by using the base quality score as

$$P ( {read}_{i} \vert NT ) = \{ \begin{matrix}1 ‐ {10}^{- \frac{B A Q}{10}} , & NT = “ A ” \\ \frac{1}{3} \times {10}^{- \frac{B A Q}{10}} , & NT \neq “ A ”\end{matrix}$$

The nucleotide with the highest posterior probability (Pmax) is used to construct the consensus read, and assign a quality to this nucleotide by using the phred score of its probability as −10 log 10(1−Pmax). The quality scores of the consensus read are rounded to the nearest integers and are stored in a bam file with ASCII characters from 33 to 126. So, the maximum phred quality score of a nucleotide is 93, which is equivalent to an error rate of <10-9.

Finally, consensus reads are exported as single-end reads, along with their base quality information into a bam file, for each individual sample. Read information such as “NUMTS” and the number of nucleotide mismatches to the rCRS or the major mtDNA sequence of the sample are exported as additional annotations in the alignment file.

### Example 13: High-Quality mtDNA Sequencing by Using STAMP

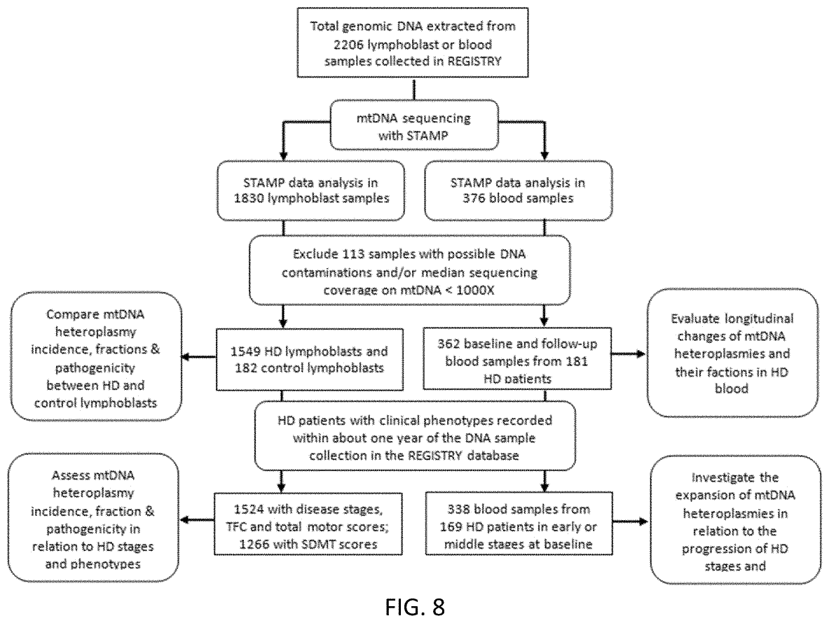

In total, the inventors prepared mtDNA sequencing libraries with STAMP for 2206 REGISTRY samples (FIG. 8). Among them, 2107 (95.5%) with a median mtDNA sequencing coverage of consensus reads greater than 1000× were used for calling heteroplasmies. The average median coverage of consensus reads on mtDNA, after quality control for heteroplasmy calling, was about 3600× in DNA from lymphoblasts and 6100× in DNA from blood samples. According to the statistical power of STAMP in discriminating true low-fraction variants from sequencing errors in mtDNA, the inventors called mtDNA heteroplasmies at variant allele fraction (VAF)≥1% in lymphoblasts and at VAF≥0.5% in blood samples, respectively.

The inventors observed that of the mtDNA heteroplasmies which passed quality control filters in 17 lymphoblasts and 320 blood samples, >95% were detectable at VAF≥0.2% in sequencing replicates performed on the same samples. The mtDNA heteroplasmies shared between sequencing replicates displayed high correlations, and no statistically significant differences in their VAFs.

### Example 14: Elevation of Pathogenic mtDNA Variant Dosages in HD Lymphoblasts

Huntington's disease (HD) is a monogenic disorder caused by the expansion of cytosine-adenine-guanine trinucleotide (CAG) repeats in the HIT gene at chromosome 4p16.3. The mutant HIT gene produces an elongated version of the huntingtin protein with an abnormally long polyglutamine tract, which leads to protein aggregation and related toxicity in cells. Although HIT is expressed in various tissues, the brain, particularly the striatum, is vulnerable to mutant huntingtin (mhtt) associated toxicity. The primary manifestations of HD include involuntary movement, impaired learning ability, and severe depression. The average age of onset of the characteristic motor symptoms is between 40 and 50 years old, followed by a progressive decline of motor, cognitive, and psychiatric functions for an average of 20 years prior to death.

The biological processes that determine the onset and progression of HD are still elusive. Recent studies suggest that mitochondrial dysfunction may be involved in HD pathogenesis. Mitochondria are subcellular organelles of eukaryotes which play vital roles in maintaining energetic and metabolic homeostasis. Evidence for mitochondrial dysfunction in HD was first reported in the post-mortem brain of HD patients, which show low mitochondrial oxidative phosphorylation (OXPHOS) protein activity and energy deficits. Mitochondrial dysfunction was further found in peripheral tissues and cell lines of HD patients, such as blood, lymphoblasts, skeletal muscle and skin fibroblasts.

Several molecular mechanisms have been proposed to connect mutant huntingtin (mhtt) to mitochondrial dysfunction. Studies in HD knock-in mice indicate that toxic fragments derived from mhtt can suppress the expression of PGC-1α, a key regulator of mitochondrial biogenesis and OXPHOS. mhtt has also been found to physically interact with mitochondria, reducing mitochondrial membrane potential. Furthermore, mhtt may stimulate mitochondrial network fragmentation, and it has recently been found to impair mitophagy, an evolutionarily conserved quality control system in eukaryotes to selectively remove dysfunctional mitochondria. Perturbation of mitochondrial tubular networks, morphology, and mitophagy are pathological features common to various neurodegenerative diseases. These mitochondrial defects, along with an imbalance of reactive oxygen species triggered by mhtt in cells, may lead to a vicious cycle that results, over time, in damage in mitochondria and ultimately cell death.

In contrast to other cellular systems, human mitochondria, especially the OXPHOS system, are encoded not only by the nuclear genome (nDNA) but also by the mitochondrial genome (mtDNA). Human mtDNA is a 16.6 kb circular DNA encapsulated in the inner membrane of mitochondria. It encodes for 22 tRNA and 2 rRNA genes used for mitochondrial protein synthesis as well as 13 evolutionarily conserved proteins in four of the five OXPHOS protein complexes. The accumulation of mutations in mtDNA of somatic tissues has been suggested as a possible driver of age-related mitochondrial dysfunction. Transgenic mice with an increased level of mtDNA mutations caused by a mutant version of the mtDNA polymerase γ manifest progeroid phenotypes and early neurodegeneration that resemble human aging. Clonal expansion of pre-existing mutations in mtDNA of somatic tissues has been shown to contribute to accelerated mitochondrial aging and OXPHOS defects in human diseases.

Because there are multiple copies of mtDNA in a single cell, mutations can arise and co-exist with wild-type mtDNA in a state called heteroplasmy, which has been linked to a variety of mitochondrial disorders in humans. A previous study from the inventors' group on lymphoblasts collected in the 1000 Genomes project indicates that about 90% of individuals in the general population carry at least one heteroplasmy in mtDNA, and purifying selection keeps most of the pathogenic heteroplasmies at a low fraction (Ye K. et al., Proc. Natl. Acad. Sci. U.S.A. 111, 10654-9 (2014)). Therefore, the ubiquity of mtDNA heteroplasmies in somatic tissues along with relaxed selective constraints caused by impaired mitochondrial dynamics and quality control under certain conditions, such as the presence of mhtt, may facilitate the increase of the fractions of heteroplasmies in cells, culminating in dysfunctional mitochondria and related energy deficits.

The inventors identified 9729 heteroplasmies at 4871 sites in mtDNA of 1731 lymphoblasts that passed quality control for heteroplasmy calling. 2790 (57%) of the heteroplasmic sites were singletons and another 1779 (37%) were rare, detected in fewer than 5 samples. The average heteroplasmy incidence of 5.6 found in the current study was higher than the incidence of 4 found in the 1085 lymphoblasts from the 1000 Genomes project which the inventors previously observed by using the whole genome sequencing data set with a lower average depth of coverage of 1805× on mtDNA.

The inventors then compared mtDNA heteroplasmies in lymphoblasts between 1549 HD patients and 182 control individuals. Since mtDNA heteroplasmies, especially pathogenic heteroplasmies, are subject to strong purifying selection in lymphoblasts, the inventors also assessed whether there was an overrepresentation of pathogenic heteroplasmies in HD lymphoblasts relative to controls. The inventors determined the pathogenicity of variants in protein-coding and RNA-coding regions of human mtDNA based on a variety of sources including known disease associations, bioinformatic pathogenicity predictions, and variant frequency in the general population.

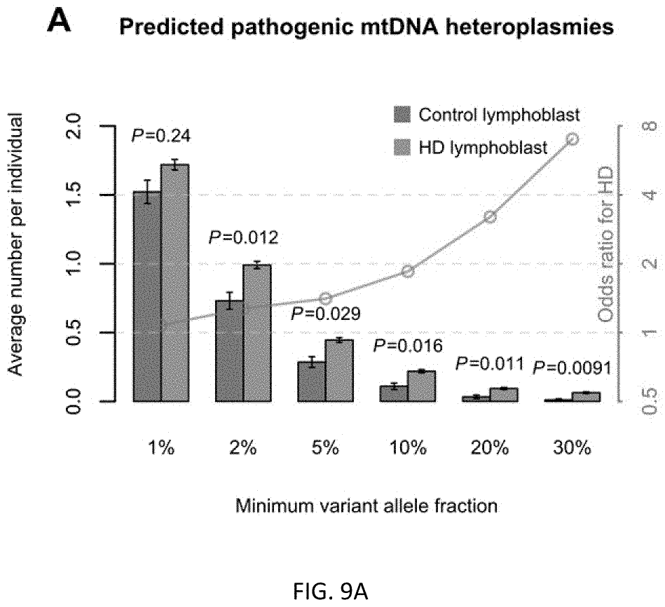

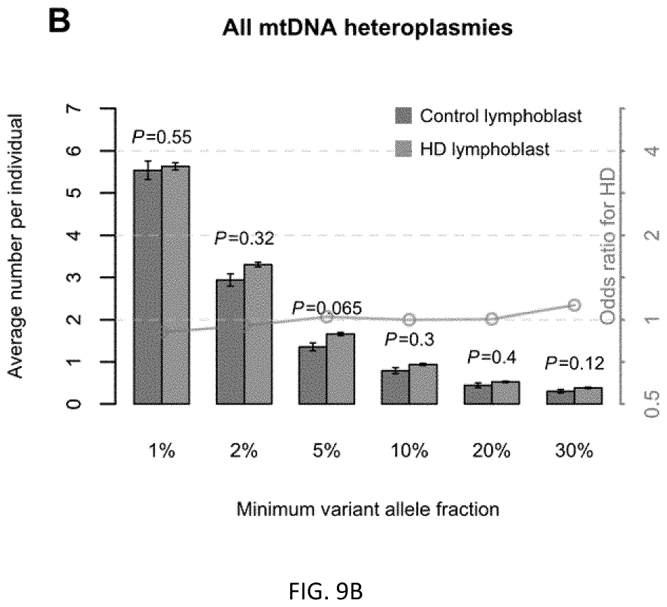

As a result, the inventors found that HD patients possessed more predicted pathogenic heteroplasmies of medium and high fractions (VAF≥2%, P=0.012) in lymphoblasts compared to control individuals (FIG. 9A). The elevation of pathogenic heteroplasmy incidence in HD lymphoblasts became more pronounced when calculated with only high-fraction heteroplasmies, showing a rise in odds ratios for HD from 1.3, when computed at VAF≥2%, to 7.0 when computed at VAF≥30% (P=0.0091, FIG. 9A) in logistic regression analyses. Similar odds ratios of predicted pathogenic heteroplasmies for HD were also attained among the subset of lymphoblasts from young and middle-aged individuals (age<55 yrs), including 887 HD patients and 138 control individuals (average age: 43.8 yrs. in patients vs 44.4 yrs. in controls). Therefore, the observed changes in the fraction distribution of heteroplasmies in HD lymphoblasts may be attributed to early to mid-life mutations in mtDNA.

Interestingly, the incidence of all mtDNA heteroplasmies and heteroplasmies that were not predicted to be pathogenic did not dramatically increase in HD lymphoblasts as compared to control lymphoblasts, regardless of their VAFs (P≥0.065, FIG. 9B). These results indicate that the overall mtDNA mutation load and fraction distribution in lymphoblasts were not affected by HD.

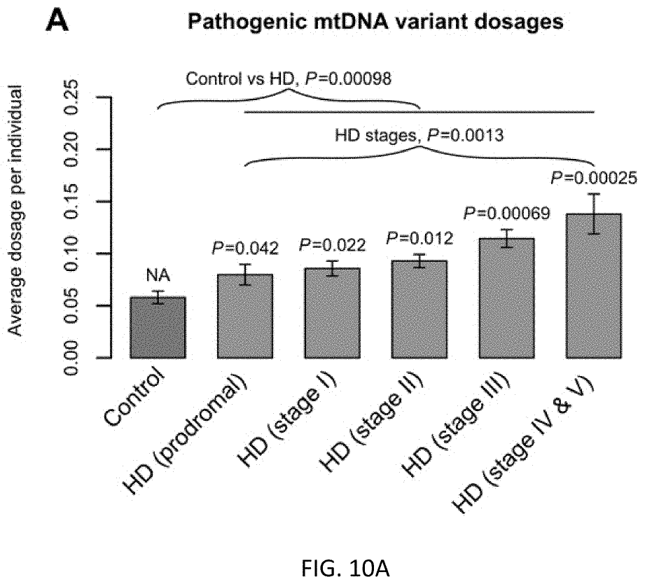

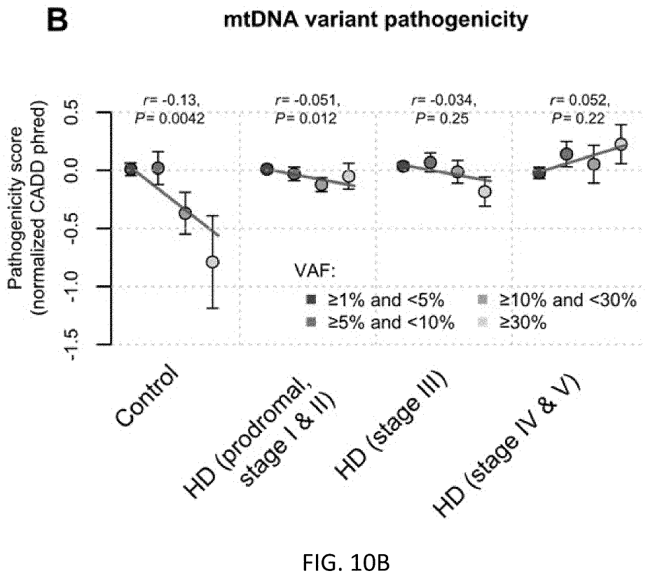

Since mutations can occur independently in different mtDNA molecules at a cellular or tissue level, the inventors computed the variant dosage of mtDNA heteroplasmies in each lymphoblast of the current study as the sum of the VAFs of all heteroplasmies identified in that sample, in order to represent the overall degree of variant load and fraction expansion in mtDNA. Again, HD lymphoblasts exhibited significantly increased dosages of predicted pathogenic heteroplasmies (P=0.00098, FIG. 10A) but similar variant dosages of all heteroplasmies and heteroplasmies without pathogenicity predications (P≥0.47), compared to control lymphoblasts.

### Example 15: Pathogenic mtDNA Variant Dosages in HD Lymphoblasts Increase with Disease Stages

Next, the inventors examined how the elevated variant dosages of predicted pathogenic heteroplasmies observed in HD lymphoblasts would relate to HD clinical stages. Among the 1549 HD patients, 1524 had information on Huntington's Disease Rating Scale (UHDRS '99) total functional capacity (TFC), total motor scores, and diagnostic confidence levels recorded in the REGISTRY clinical database within about 1 year of the sample collection. 156 were in the prodromal stage (UHDRS diagnostic confidence level<4). The remaining 1368 patients were grouped into different disease stages based on their TFC scores. 766, 404 and 198 of them were in early (I: TFC score≥11; II: 7≤TFC score<11), middle (III: 4≤TFC score<7), and late stages (IV/V: TFC score≤3), respectively.

Of note, substantial increases in the variant dosages of predicted pathogenic mtDNA heteroplasmies had already been revealed in lymphoblasts of HD patients in prodromal and early stages (logistic regression adjusted for age, sex, and sequencing coverage, P≤0.042, FIG. 10A), which became more and more prominent among patients in middle and late stages (P≤0.00069). Accordingly, there was a significant association between pathogenic mtDNA variant dosages in lymphoblasts and advancing disease stages among the 1524 HD patients (P=0.0013) as well as among the 1368 manifest HD patients (P=0.0071, FIG. 10A).

The increase of pathogenic mtDNA variant dosages with disease stages could result from a relaxation of purifying selection on mtDNA heteroplasmies in HD lymphoblasts. In controls, the inventors found a negative correlation between the VAFs of nonsynonymous heteroplasmies, which can alter the amino acid sequences of OXPHOS protein complexes, and their CADD pathogenicity scores (r=−0.13, P=0.0042, FIG. 10B). The pathogenicity scores of nonsynonymous heteroplasmies in controls were also significantly lower than those of all possible nonsynonymous variants in mtDNA (Mann-Whitney U test, P=0.001). These results substantiate the inventors' observations from the general population, indicating that purifying selection may prevent the expansion of pathogenic heteroplasmies in lymphoblast mtDNA.

In contrast, the degree of purifying selection on mtDNA heteroplasmies diminished in HD patients in prodromal, early and middle stages, with mild negative correlations between the VAFs of nonsynonymous heteroplasmies and their pathogenicity (r=−0.51˜-0.034, FIG. 10B). In late-stage HD patients, the inventors even detected a slight positive correlation of pathogenicity with the VAFs of nonsynonymous heteroplasmies (r=0.052, FIG. 10B), suggesting a complete loss of purifying selection on mtDNA heteroplasmies in late stages of the disease.

The observed increases in pathogenic variant dosages and the relaxation of purifying selection on mtDNA heteroplasmies in HD lymphoblasts also persisted among young and middle-aged individuals. Taken together, these results indicate that the decline of mtDNA quality could be a molecular signature underlying the progression of HD stages.

### Example 16: Pathogenic mtDNA Variant Dosages in HD Lymphoblasts are Linked to Clinical Phenotypes and Disease Burden

Progression of HD is largely determined by CAG repeats in HIT, and is characterized by deterioration of motor, cognitive and psychiatric functions. The inventors thus sought to investigate how predicted pathogenic mtDNA heteroplasmies in lymphoblasts of HD patients could reflect these functional declines and correspond to HIT′ related genetic burden. In addition to UHDRS TFC and total motor scores, the inventors retrieved UHDRS symbol digit modalities test scores (SDMT, N=1266) from the REGISTRY database to assess the severity of cognitive signs in HD patients.

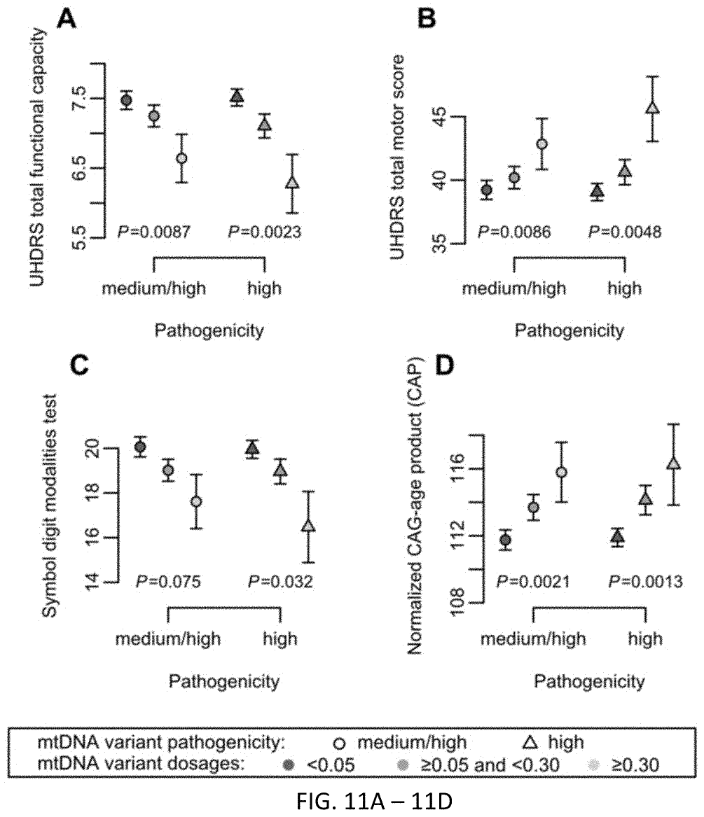

By using linear regression with adjustment for age, sex, sequencing coverage, and CAG repeat length, it was found that pathogenic mtDNA variant dosages in lymphoblasts displayed significant associations with disease severity, as measured on functional capacity (P=0.0087, FIG. 11A) and motor scales (P=0.0086, FIG. 11B), and a suggestive association with SDMT scores (P=0.075, FIG. 11C). These associations were strengthened when the inventors focused on mtDNA heteroplasmies predicted to have high pathogenicity (P=0.0023-0.032, FIGS. 11A-11C), which supports the conclusion that the loss of selective constraints on mtDNA during HD progression leads to elevated pathogenicity of mtDNA heteroplasmies.

Furthermore, the inventors noted significant correlations between pathogenic mtDNA variant dosages and HD disease burden which the inventors computed as a normalized product between CAG repeat length and age (linear regression adjusted for sex and sequencing coverage, P≤0.0021, FIG. 11D). Inspired by such an observation, the inventors subsequently assessed age-dependent changes in mtDNA heteroplasmies by using linear models comprising age, CAG repeat length and their interaction as predictors for mtDNA heteroplasmies in HD lymphoblasts. As a result, the inventors found that the interaction effect of elongated CAG repeat length and advancing age was positive, and significant in predicting both variant dosages (P≤0.011) and incidence (P≤0.016) of predicted pathogenic heteroplasmies in addition to the effect of age (P≤0.0045, Table 2). Of note, expanded CAG repeat length also showed a substantial main impact on the increase of variant dosages (P=0.031) and incidence (P=0.037) of heteroplasmies predicted to have high pathogenicity (Table 2). In contrast, neither CAG repeat length nor its interaction with age was found to affect variant dosages or incidence of heteroplasmies that were not predicted to have medium or high pathogenicity (P≥0.23), while the effects of age remained significant (P≤7.4×10⁻⁵, Table 2).

TABLE 2
Age- and CAG-dependent changes of mtDNA heteroplasmies in lymphoblasts of HD patients.

| | mtDNA | | | CAG | | Age × CAG | |
|---|---|---|---|---|---|---|---|
| | variant | Age | | repeat length | | repeat length | |
| Variables | pathogenicity | Beta (SE) | P | Beta (SE) | P | Beta (SE) | P |
| mtDNA | M/H | 0.012 | 0.00011 | 0.018 | 0.15 | 0.0014 | 0.011 |
| variant | | (0.003) | | (0.012) | | (0.0006) | |
| dosages | H | 0.012 | 5.5 × 10⁻⁵ | 0.026 | 0.031 | 0.0015 | 0.0065 |
| | | (0.003) | | (0.012) | | (0.0005) | |
| | others | 0.013 | 7.4 × 10⁻⁵ | 0.003 | 0.82 | 0.0003 | 0.55 |
| | | (0.003) | | (0.013) | | (0.0006) | |
| mtDNA | M/H | 0.009 | 0.0045 | 0.014 | 0.26 | 0.0014 | 0.013 |
| variant | | (0.003) | | (0.012) | | (0.0005) | |
| incidence | H | 0.010 | 0.00061 | 0.024 | 0.037 | 0.0013 | 0.016 |
| | | (0.003) | | (0.012) | | (0.0005) | |
| | others | 0.015 | 4.8 × 10⁻⁶ | 0.002 | 0.87 | 0.0007 | 0.23 |
| | | (0.003) | | (0.013) | | (0.0006) | |

The variant incidence and dosages of mtDNA heteroplasmies were inverse normal transformed and were further adjusted for sex and sequencing coverage. The associations were assessed by using the model: INV dosage/incidence˜age+CAG_length+age×CAG_length. M/H: medium or high pathogenicity; H: high pathogenicity; others: not predicted with medium or high pathogenicity. P values <0.05 are highlighted in bold type.

Compared to HD lymphoblasts, control lymphoblasts displayed a slower age-dependent increase in the variant dosages and incidence of predicted pathogenic heteroplasmies (P≥0.3, implying that the expansion of heteroplasmies with damaging consequences is largely suppressed in lymphoblasts of healthy individuals expressing normal HTT. Collectively, these results indicate that elongated CAG repeats in HTT may accelerate the age-dependent increase of pathogenic heteroplasmies in lymphoblasts, echoing the biochemical evidence that mhtt impairs mitochondrial quality control and causes bioenergetic deficits.

### Example 17: Expansion of Pre-Existing mtDNA Heteroplasmies in Blood of HD Patients

Although lymphoblasts provide a valuable genetic resource for studying patients' mutations, the inventors are unable to rule out the possibility that Epstein-Barr virus induced B lymphocyte transformation could create new heteroplasmies or change the fractions of existing heteroplasmies in mtDNA. The inventors also did not know whether the observed changes of mtDNA heteroplasmies in HD samples were due to a rapid rise of new heteroplasmies or an expansion of existing heteroplasmies during HD progression.

To partially resolve these issues, the inventors performed STAMP on longitudinal blood samples from 188 HD patients collected from two visits 5-9 years apart (median=6 years) in REGISTRY to directly investigate changes in mtDNA heteroplasmies during HD progression. The inventors further hypothesized that changes in pathogenic mtDNA heteroplasmies during the follow-up would be associated with the degree of disease progression among these patients. The inventors called mtDNA heteroplasmies at VAF≥0.5% in these samples. The inventors found that mtDNA from 7 individuals showed an excess of heteroplasmies (N≥14) at known polymorphic sites of mtDNA, which could be caused by low-level contamination with other DNA samples. As such, the inventors focused on the remaining 181 HD patients for the following analysis, of whom 169 were not in HD late stages at baseline.

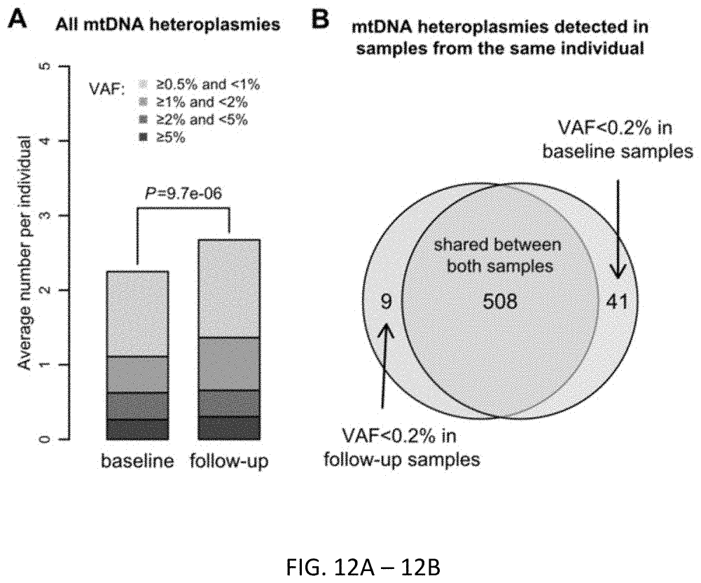

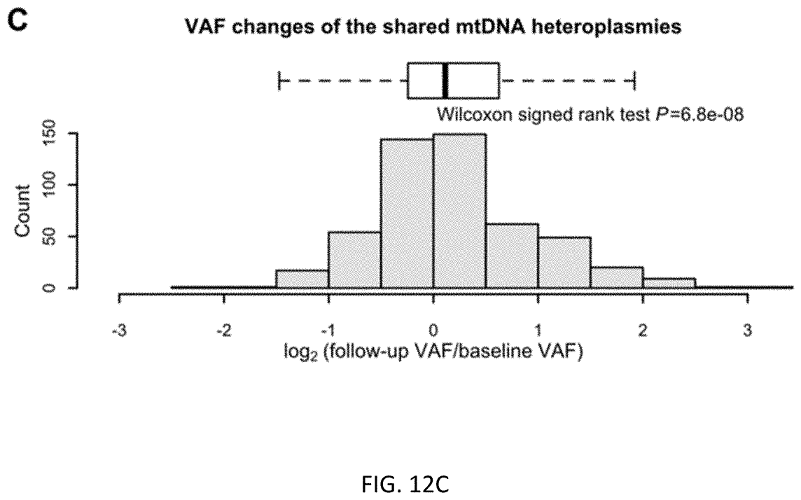

The inventors observed a roughly 19% incidence increase of heteroplasmies from an average of 2.25 at baseline to an average of 2.67 at follow-up (paired t-test, P=9.7×10⁻⁶, FIG. 12A). Among the 558 mtDNA heteroplasmies with VAF≥0.5%, 508 (91%) and 529 (95%) could be detected in both baseline and follow-up samples from the same individual at lower VAFs of ≥0.2% and ≥0.1%, respectively (FIG. 12B). Given the known false discovery rate of STAMP in calling heteroplasmies, the inventors used a VAF of ≥0.2% in both samples to define pre-existing heteroplasmies in the following analyses. 91% (407/449) of the heteroplasmic sites were detected in only one of the 181 HD patients, suggesting that they were not recurrent mutations driven by a selective advantage in the hematopoietic system. There was also a high correlation in VAFs of heteroplasmies detected between the baseline and follow-up samples (r>0.99, P<2.2×10⁻¹⁶). Therefore, most heteroplasmies detected in the follow-up samples must already have existed in the hematopoietic system, specifically, in long-lived stem or progenitor cells, since the time of the baseline visit.

Interestingly, the results reveal that the increase of detectable heteroplasmies in follow-up samples can largely be attributed to the expansion of pre-existing mtDNA heteroplasmies in the hematopoietic system. The inventors found a statistically significant increase of VAFs of these pre-existing heteroplasmies when comparing their VAFs between the baseline and follow-up samples (Wilcoxon signed rank test, P=6.8×10⁻⁸, FIG. 12C). This increase might be linked to the advance of age or disease stage among these HD patients.

### Example 18: Expansion of Pathogenic mtDNA Heteroplasmies in Blood Parallels the Progression of HD Stages and Clinical Phenotypes

To investigate the influence of HD progression on the expansion of pre-existing mtDNA heteroplasmies, the inventors divided the 169 HD patients who were not in late stages at baseline into two groups, including 134 who experienced progression of disease stage at follow-up, and 35 showing a slow progression of the disease with a stable stage at follow-up. The inventors detected 359 pre-existing heteroplasmies in 120 progressed-stage patients and 107 in 28 stable-stage patients. Of them, 56 and 16 heteroplasmies were predicted to be pathogenic in 38 progressed-stage patients and 13 stable-stage patients, respectively.

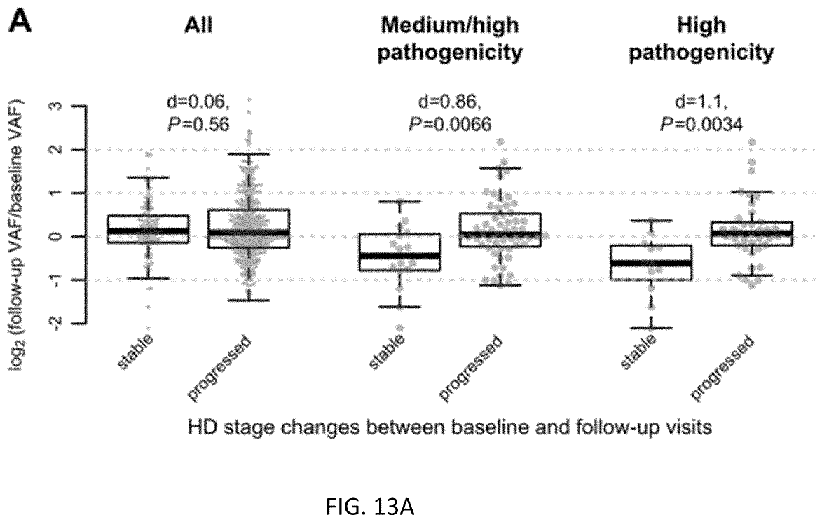

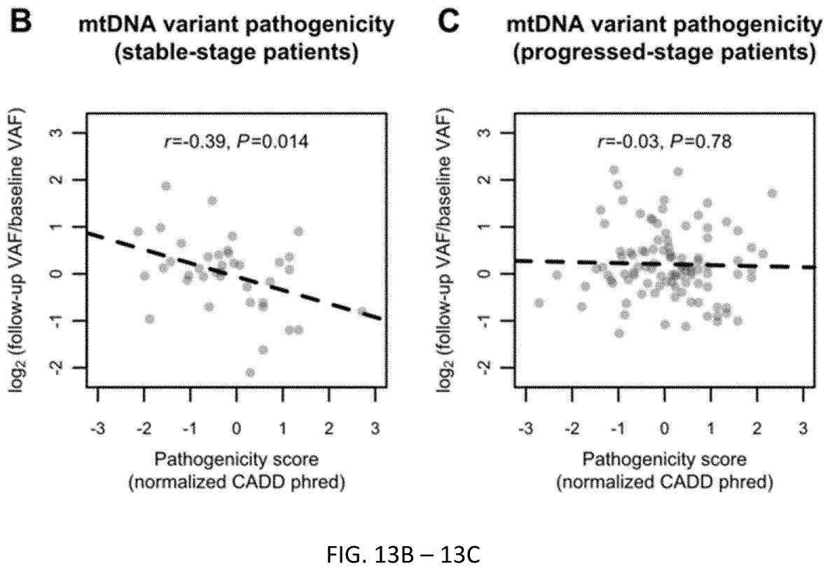

Among all the pre-existing heteroplasmies, the degree of VAF changes at follow-up was comparable in progressed-stage patients and stable-stage patients (Cohen's d=0.06; t-test, P=0.56, FIG. 13A). In contrast, the VAF changes of the 72 predicted pathogenic heteroplasmies displayed a significant difference between these two patient groups (Cohen's d≥0.86; t-test, P≤0.0066, FIG. 13A). As reassuring evidence that the pathogenicity of these heteroplasmies, rather than other uncontrolled confounding factors, contributed to the observed difference, the inventors found that the VAF changes of heteroplasmies without pathogenicity annotations did not correspond to the stage changes among the 51 patients (Cohen's d=−0.05; t-test, P=0.78) carrying pathogenic heteroplasmies.

In stable-stage patients, predicted pathogenic heteroplasmies exhibited a decrease in their VAFs at follow-up (Wilcoxon signed rank test, P≤0.029), suggesting effective purifying selection on mtDNA. The inventors also noted a negative correlation between the degree of VAF changes and the pathogenicity (CADD phred scores) of nonsynonymous heteroplasmies (r=−0.39, P=0.014, FIG. 13B) in stage-stage patients, indicating that heteroplasmies with a higher pathogenic potential were subject to stronger purifying selection. The evidence for purifying selection on mtDNA disappeared among progressed-stage patients, displaying slightly increased VAFs of pathogenic heteroplasmies at follow-up (Wilcoxon signed rank test, P≥0.23) and a weak correlation between the VAF changes and pathogenicity of nonsynonymous heteroplasmies (r=−0.03, FIG. 13C). These results suggest that loss of selective constraints on pre-existing heteroplasmies in mtDNA may contribute to the expansion of their fractions during HD progression.

Next, the inventors assessed how the expansion of predicted pathogenic heteroplasmies in blood would relate to clinical phenotypic data recorded at baseline and follow-up visits of these patients. By using mixed-effects regression modeling of variant expansion in longitudinal blood samples, the inventors did not find evidence that the baseline HD-related clinical phenotypes influenced the degree of expansion of predicted pathogenic heteroplasmies (P≥0.12, Table 3), which demonstrates that the differences in the changes of their VAFs were not secondary to the individual variation in disease severity at baseline.

TABLE 3
Associations of mtDNA variant fraction changes in blood with HD clinical phenotypes.

| | mtDNA | | | | | | |
|---|---|---|---|---|---|---|---|
| | variant | | | | | | |
| | pathogenicity | TFC score | | Total motor score | | SDMT score | |
| Variables | (N)* | Beta (SE) | P | Beta (SE) | P | Beta (SE) | P |
| Model 1 | M/H (72) | 0.005 | 0.90 | 0.010 | 0.20 | 0.009 | 0.41 |
| (baseline | | (0.037) | | (0.008) | | (0.011) | |
| phenotypes) | H (49) | 0.033 | 0.49 | 0.018 | 0.12 | 0.015 | 0.25 |
| | | (0.047) | | (0.011) | | (0.013) | |
| | others | −0.023 | 0.38 | 0.002 | 0.70 | −0.007 | 0.42 |
| | (145) | (0.026) | | (0.006) | | (0.009) | |
| Model 2 | M/H (72) | −0.13 | 0.0011 | 0.015 | 0.021 | −0.050 | 0.00046 |
| (follow-up | | (0.037) | | (0.006) | | (0.013) | |
| phenotypes) | H (49) | −0.15 | 0.0044 | 0.019 | 0.024 | −0.042 | 0.011 |
| | | (0.050) | | (0.008) | | (0.015) | |
| | others | −0.014 | 0.62 | 0.001 | 0.83 | −0.002 | 0.92 |
| | (145) | (0.029) | | (0.005) | | (0.015) | |

The associations were assessed by using the following linear mixed-effects model: log 2(VAF follow-up/VAF baseline)—score+age+sex+CAG_length+followup_duration+(1|patient_id). In the analyses of the follow-up clinical phenotypes, the baseline phenotype and the baseline disease stage were considered as additional fixed-effect covariates in the model. *The number of mtDNA heteroplasmies used for analysis; due to missing phenotypes in either the baseline sample or the follow-up sample, 1 pathogenic heteroplasmy and 2 non-pathogenic heteroplasmies were not included in the analyses of total motor scores, and 2 pathogenic heteroplasmies and 32 non-pathogenic heteroplasmies were not included in the analyses of SDMT scores; M/H: medium or high pathogenicity; H: high pathogenicity; others: not predicted with medium or high pathogenicity in HD patients carrying pathogenic heteroplasmies. P values <0.05 are highlighted in bold type.

In contrast, significant associations were consistently detected between the VAF changes of predicted pathogenic heteroplasmies and the follow-up clinical phenotypes, including TFC scores (P≤0.0044), total motor scores (P≤0.024) and SDMT scores (P≤0.011), with adjustment for the baseline phenotype and the baseline disease stage (Table 3). Moreover, no apparent associations were found between the clinical phenotypes and the VAF changes of other heteroplasmies detected in the 51 patients carrying pathogenic heteroplasmies (Table 3). These results point to an overall decline of mtDNA quality in the hematopoietic system during the clinical progression of HD which is independent of the effect of normal aging on heteroplasmy expansion.

Furthermore, the inventors noted a significant positive effect of CAG repeat length on the VAF changes of 29 pathogenic heteroplasmies (linear mixed-effect model, P=0.0034) identified among early-stage patients with moderate motor symptoms at baseline (TFC score≥7 and total motor score<25), which was in contrast to no CAG-related expansion of other heteroplasmies in these patients. But this effect was weakened and became insignificant among all patients with longitudinal blood samples in the current study, implying that HIT may exert its impact on mtDNA early in HD pathogenesis. These results further illustrate that elongated CAG repeats in HIT may promote the expansion of pathogenic heteroplasmies during HD progression, as opposed to the suppression of their factions via effective mitochondrial quality control during normal aging.

### Example 19

In the current study, the inventors applied ultra-deep mtDNA targeted sequencing to assessing mtDNA heteroplasmies in lymphoblasts as well as longitudinal blood samples of HD patients. With a refined design, STAMP provides a low error rate of 0.02% per base of mtDNA as well as >97% sensitivity in identifying mtDNA heteroplasmies of VAF≥0.5-1.0%.

Age-dependent accumulation of mtDNA heteroplasmies in blood has been reported in cross-sectional studies, showing an increase of about 1% per year of heteroplasmies with VAF≥2% in the general population. The present data in longitudinal blood samples indicate that this age-related increase of heteroplasmy incidence is largely due to the expansion of pre-existing heteroplasmies in the hematopoietic system, rather than an increased mtDNA mutation rate possibly associated with aging.

The instant data provides evidence to support the existence of purifying selection on mtDNA heteroplasmies, which could be an important mechanism to ensure cellular mitochondrial function during aging. In lymphoblasts, the minor effects of low-fraction pathogenic heteroplasmies on HD may illustrate the mitochondrial threshold effect, whereby cells can tolerate low-fraction, recessive heteroplasmies in mtDNA without manifesting the associated phenotypic defects and triggering the quality control system to purge them. The increased pathogenic mtDNA variant dosages in HD and their positive association with disease severity indicate that such a quality control system is impaired in HD. This defective mtDNA quality control was also suggested in longitudinal blood samples of HD patients whose clinical phenotypes progressed over time, showing an expansion of pre-existing pathogenic heteroplasmies, probably early or midlife mutations in mtDNA, in the hematopoietic system. The inventors further performed sensitivity analyses and found that the results were not affected by possibly fixed pathogenic heteroplasmies in lymphoblasts as well as pre-existing very-low-fraction pathogenic heteroplasmies in blood samples.

In the instant disclosure, the inventors noted increased incidence and fractions of mtDNA heteroplasmies in lymphoblasts compared to blood samples. It agrees with the results from previous cell studies, which showed higher numbers of heteroplasmies in mtDNA of skin fibroblasts, colonic epithelial cells, and induced pluripotent stem cells than those of the parental tissues. Recently, the prevalence and propagation of mtDNA heteroplasmies have been demonstrated in hematopoietic cells using various single-cell sequencing technologies. These technological advances will provide an unprecedented opportunity to study the changes of mtDNA at a single cell level and their impact on cellular phenotypes in HD and other age-related diseases.

The data suggest that elongated CAG repeats in HIT may affect the mitochondrial quality control system. In line with this finding, it has been demonstrated in numerous cellular and animal models of HD that mhtt can impair mitophagy, through disruption of the autophagic receptor p62-mediated cargo recognition, limiting the transport and fusion of autophagosome to lysosome, and recruitment of valosin-containing protein to mitochondria. Moreover, mhtt has been implicated in the biological pathways that regulate mitochondrial morphology and tubular networks, sinceit may bind to the mitochondrial fission protein, drp1, and alter the levels of fusion proteins to favor mitochondrial fission over fusion. Changes in mitochondrial fission and fusion dynamics and loss of effectiveness of the mitophagic apparatus in identifying and removing dysfunctional mitochondria may collectively relax selective constraints on mtDNA, precipitating the expansion of pathogenic mtDNA heteroplasmies in cells.

Moreover, the instant results imply that HTT-related genetic burden may not completely account for the impairment of mitochondrial quality control. Other modifiers of HD progression may also play a role in this process. Of note, the genome-wide association study conducted by the GeM-HD Consortium identified associations of age at onset of motor symptoms with genetic variants in the mitochondrial fission pathway and mtDNA regulation, pointing to a possible interaction between nDNA-encoded mitochondrial genes and mtDNA in the pathogenesis and progression of HD.

In addition to HTT's role in mitochondrial quality control, HTT has been investigated in the context of other mitochondrial characteristics, such as mitochondrial biogenesis and oxidative damage. In lymphoblast and blood samples, the inventors measured mtDNA content in relation to the amount of nuclear DNA as a proxy for mitochondrial biogenesis by using both STAMP and a quantitative PCR-based method. The inventors found that mtDNA content did not correlate with the variant dosages of predicted pathogenic heteroplasmies in lymphoblasts, or with the expansion of pre-existing pathogenic heteroplasmies in blood samples of HD patients. Therefore, mhtt's impact on mtDNA quality control may be independent of its impact on mitochondrial biogenesis.

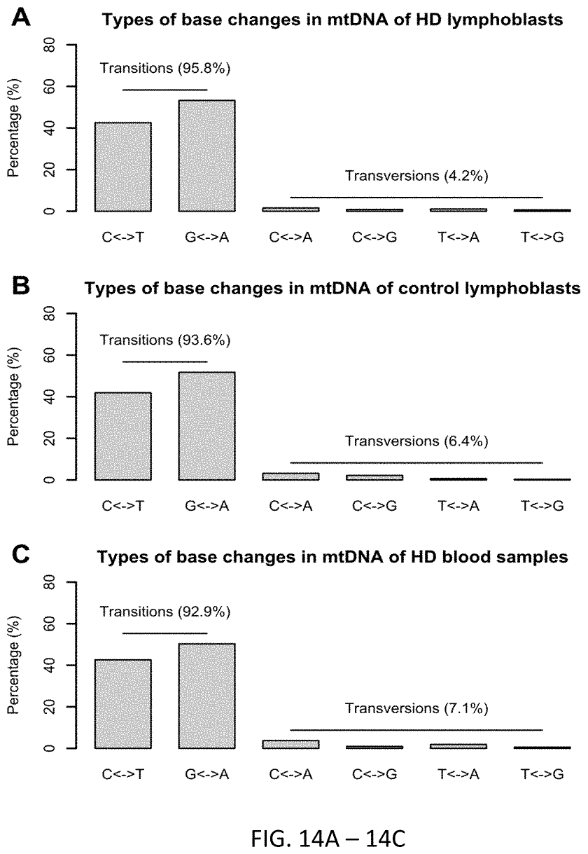

The decline of mtDNA quality in HD lymphoblasts and blood samples may not be consequence of oxidative damage to mitochondria in HD. The inventors found similar patterns of base changes of mtDNA heteroplasmies in lymphoblasts and blood samples of HD patients compared to lymphoblasts of control individuals, with high transition to transversion ratios of >13 (FIGS. 14A-14C). The minimal proportions of transversion base changes in mtDNA of HD samples are suggestive of replication errors or base deamination in mtDNA rather than damage associated with oxidative stress, consist with the somatic mutation pattern of mtDNA identified in recent human studies. Indeed, oxidative stress could result from reactive oxygen species produced by defective electron transport complexes in OXHPOS system, which are partially encoded by mtDNA.

These lines of evidence further stress the importance of mitochondrial quality control during the clinical progression of HD. Intriguingly, effective purifying selection on mtDNA heteroplasmies was detected among patients with a slow disease progression in the analysis of blood samples (FIG. 13A-13C). It suggests that the decline of mtDNA quality is not an irreversible process in HD. Modulating mitochondrial network dynamics and mitophagy pathway genes, such as DRP1, HDAC6, PINK1 and GAPDH, has been shown to effectively correct mhtt-associated toxicity in cell and animal models. The interpretation of a causal role for blood mtDNA heteroplasmies in the pathogenesis of HD is limited in the current study. However, peripheral blood and related cell lines have been repeatedly used as a surrogate for studying HD's impact. Peripheral blood of HD patients also reveals transcriptomic changes resembling those of striatum and prefrontal cortex. Interestingly, energy metabolism is one of the significantly downregulated pathways that are shared between brain and blood, and correlates with HD severity.

In sum, the instant large-scale deep-sequencing study illustrates mtDNA changes in the hematopoietic system during HD progression, echoing a theme of defective mitochondrial quality control in HD supported by previous biochemical evidence. This study provides an accessible biomarker for HD progression and related clinical phenotypes, by harnessing mtDNA in peripheral tissues.

Table 4: Information of the EL Probes and their target regions used in STAMP. The start and end positions of the target regions are shown with those in rCRS and nuclear genome (assembly GRCh38). The ligation arm of B10 was designed with a degenerate base S (G/C) to match the mtDNA sequence with an 8271-8279 or 8281-8289 deletion.

*mtDNA polymorphisms were obtained from the MITOMAP website. EUR: polymorphisms in the macro-haplogroups of Europeans (H, U, J, T, K, R, V, I, W, X, and N); AFR: polymorphisms in the macro-haplogroups of Africans (L); ASN: polymorphisms in the macro-haplogroups of Asians (M, B, D, C, A, F, E, G, Q, Z, Y, P, S, and O); only polymorphisms having a population frequency >1% are listed; the polymorphisms are shown with the first letter indicating the reference allele of rCRS, followed by the position and the variant allele.

TABLE 4
Information of the EL Probes and their target regions used in STAMP.

| | | | | Ligation | Extension | Max | Ligation | Extension | | | |
|---|---|---|---|---|---|---|---|---|---|---|---|
| | | | | arm | arm | barcode | arm SEQ | arm SEQ | mtDNA polymorphisms in | | |
| Probe ID | Chr. | Start | End | strand | strand | length | ID NO | ID NO | EUR | AFR | ASN |
| A1 | chrM | 311 | 753 | + | − | 15 | 4 | 5 | C315CC; | C315CC; | C315CC; |
| | | | | | | | | | A750G | G316A; | A750G; |
| | | | | | | | | | | C325T; | C752T |
| | | | | | | | | | | A750G | |
| A2 | chrM | 701 | 1141 | − | + | 15 | 6 | 7 | G709A | G709A; | G709A |
| | | | | | | | | | | T710C; | |
| | | | | | | | | | | G719A | |
| A3 | chrM | 1095 | 1535 | + | − | 15 | 8 | 9 | | | T1107C |
| A4 | chrM | 1474 | 1911 | − | + | 12 | 10 | 11 | | | |
| A5 | chrM | 1854 | 2283 | + | − | 15 | 12 | 13 | | | |
| A6 | chrM | 2206 | 2646 | − | + | 15 | 14 | 15 | | A2220G | |
| A7 | chrM | 2538 | 2980 | + | − | 12 | 16 | 17 | | | |
| A8 | chrM | 2932 | 3377 | − | + | 15 | 18 | 19 | | | |
| A9 | chrM | 3313 | 3753 | + | − | 15 | 20 | 21 | C3741T | | G3316A |
| A10 | chrM | 3687 | 4128 | − | + | 15 | 22 | 23 | | G3693A | T4117C |
| A11 | chrM | 4022 | 4471 | + | − | 15 | 24 | 25 | | C4025T; | |
| | | | | | | | | | | A4044G; | |
| | | | | | | | | | | T4454A | |
| A12 | chrM | 4348 | 4797 | − | + | 15 | 26 | 27 | A4793G | | |
| B1 | chrM | 4705 | 5150 | + | − | 12 | 28 | 29 | G5147A | A4722G; | A4715G; |
| | | | | | | | | | | G5147A | G5147A |
| B2 | chrM | 5099 | 5498 | − | + | 15 | 30 | 31 | | | T5108C |
| B3 | chrM | 5453 | 5896 | + | − | 15 | 32 | 33 | G5460A; | G5460A; | G5460A; |
| | | | | | | | | | G5471A | G5471A | T5465C |
| B4 | chrM | 5843 | 6286 | − | + | 15 | 34 | 35 | | | A5843G |
| B5 | chrM | 6218 | 6658 | + | − | 15 | 36 | 37 | T6221C | T6221A; | |
| | | | | | | | | | | T6221C | |
| B6 | chrM | 6514 | 6960 | − | + | 15 | 38 | 39 | | T6524C | C6960T |
| B7 | chrM | 6914 | 7357 | + | − | 15 | 40 | 41 | | G6917A; | |
| | | | | | | | | | | G7337A | |
| B8 | chrM | 7232 | 7676 | − | + | 12 | 42 | 43 | | T7660C | |
| B9 | chrM | 7608 | 8036 | + | − | 15 | 44 | 45 | | T7624A; | G8020A; |
| | | | | | | | | | | G8027A | G8027A |
| B10 | chrM | 7882 | 8297 | − | + | 15 | 46 | 47 | | T8279C | T8279C |
| B11 | chrM | 8249 | 8666 | + | − | 15 | 48 | 49 | G8251A; | G8251A; | G8251A |
| | | | | | | | | | G8269A | C8655T | |
| B12 | chrM | 8544 | 8968 | − | + | 15 | 50 | 51 | | G8545A | C8964T |
| C1 | chrM | 8912 | 9346 | + | − | 15 | 52 | 53 | | | |
| C2 | chrM | 9274 | 9695 | − | + | 15 | 54 | 55 | | | |
| C3 | chrM | 9500 | 9940 | + | − | 15 | 56 | 57 | | T9509C; | |
| | | | | | | | | | | G9932A | |
| C4 | chrM | 9353 | 10293 | − | + | 15 | 58 | 59 | | A9855G; | |
| | | | | | | | | | | A10286G | |
| C5 | chrM | 10209 | 10648 | + | − | 15 | 60 | 61 | | T10640C | |
| C6 | chrM | 10600 | 11022 | − | + | 15 | 62 | 63 | | | T10609C |
| C7 | chrM | 10926 | 11368 | + | − | 12 | 64 | 65 | | C10939T | |
| C8 | chrM | 11298 | 11744 | − | + | 15 | 66 | 67 | T11299C | T11299C; | |
| | | | | | | | | | | C11302T | |
| C9 | chrM | 11687 | 12127 | + | − | 15 | 69 | 69 | | T12122C; | G11696A |
| | | | | | | | | | | G12127A | |
| C10 | chrM | 12075 | 12502 | − | + | 15 | 70 | 71 | G12501A | G12501A | T12091C |
| C11 | chrM | 12448 | 12894 | + | − | 12 | 72 | 73 | | | C12822T |
| C12 | chrM | 12823 | 13270 | − | + | 15 | 74 | 75 | | | T13254C; |
| | | | | | | | | | | | A13263G |
| D1 | chrM | 13214 | 13658 | + | − | 15 | 76 | 77 | | C13650T; | |
| | | | | | | | | | | A13651G | |
| D2 | chrM | 13592 | 14008 | − | + | 15 | 78 | 79 | | T14000A; | |
| | | | | | | | | | | A14007G | |
| D3 | chrM | 13955 | 14396 | + | − | 15 | 80 | 81 | A13966G | G13958C | |
| D4 | chrM | 14348 | 14773 | − | + | 15 | 82 | 83 | C14766T | A14755G; | C14766T |
| | | | | | | | | | | C14766T; | |
| | | | | | | | | | | A14769G | |
| D5 | chrM | 14719 | 15163 | + | − | 16 | 84 | 85 | | | |
| D6 | chrM | 15092 | 15535 | − | + | 12 | 86 | 87 | | G15110A; | C15535T |
| | | | | | | | | | | T15514C | |
| D7 | chrM | 15353 | 15778 | + | − | 15 | 88 | 89 | | A15766G | |
| D8 | chrM | 15733 | 16178 | − | + | 12 | 90 | 91 | A16162G; | C15735T; | A15746G; |
| | | | | | | | | | A15163G; | A16166C; | A16162G; |
| | | | | | | | | | T16172C | C16167T; | T16172C |
| | | | | | | | | | | C16168T; | |
| | | | | | | | | | | C16169T; | |
| | | | | | | | | | | T16169T; | |
| | | | | | | | | | | T16172C; | |
| | | | | | | | | | | C16176T | |
| D9 | chrM | 16112 | 16561 | + | − | 15 | 92 | 93 | T16126C; | C16114A; | T16126C; |
| | | | | | | | | | G16129A; | T16124C; | G16129A; |
| | | | | | | | | | G16129C | T16126C; | T16136C |
| | | | | | | | | | | G16129A | |
| D10 | chrM | 16499 | 358 | − | + | 15 | 94 | 95 | T16519C | T16519C; | T16519C |
| | | | | | | | | | | A357G | |
| EMC1 | chr1 | 19563554 | 19563970 | + | − | 15 | 96 | 97 | | | |
| WRN | chr8 | 33162768 | 33163207 | − | + | 15 | 98 | 99 | | | |
| SERPINA1 | chr14 | 94430150 | 94439625 | − | + | 15 | 100 | 101 | | | |
| B2M | chr15 | 44723813 | 44724050 | − | + | 15 | 102 | 103 | | | |
| AXL | chr19 | 40978400 | 40978829 | + | − | 15 | 104 | 105 | | | |

What is claimed is:

1. A probe set comprising:
a first probe subset comprising a plurality of probe pairs and a second probe subset comprising a plurality of probe pairs,
wherein each probe pair within each probe subset comprises a ligation probe and an extension probe,
wherein each probe pair in the first probe subset comprises probes that anneal to the heavy strand of a mitochondrial genomic DNA and each probe pair in the second probe subset comprises probes that anneal to the light strand of a mitochondrial genomic DNA,
wherein each probe pair defines a target region of the mitochondrial genomic DNA that is not identical to any other target region defined by any other probe pair,
wherein the target regions defined by the first probe subset and the target regions defined by the second probe subset in combination cover the entirety of the mitochondrial genomic DNA,
wherein each ligation probe comprises a first primer annealing sequence and a 5′-phosphorylated ligation arm that is substantially complementary to a first end of the target region on the mitochondrial genomic DNA defined by the probe pair,
wherein each extension probe comprises an extension arm that is substantially complementary to a second end of the target region on the mitochondrial genomic DNA defined by the probe pair, and a second primer annealing sequence,
and wherein the ligation arm does not anneal to an identical or overlapping sequence on the mitochondrial genomic DNA with the extension arm.

2. The probe set of claim 1, wherein the probe pairs in the probe subsets are designed such that neighboring target regions in the heavy strand defined by the probe pairs in the first probe subset overlap with neighboring complementary target regions in the light strand defined by the probe pairs in the second probe subset.

3. The probe set of claim 2, wherein a target region in the heavy strand defined by a probe pair from the first probe subset is followed by an overlapping target region in the light strand defined by a probe pair from the second probe subset.

4. The probe set according to any one of claims 1-3, wherein each probe pair anneals to a target region that is between 200-600 nucleotides, 300-500 nucleotides, or 399-449 nucleotides in length.

5. The probe set according to any one of claims 1-4, wherein all the ligation probes comprise a common nucleotide sequence for the first primer annealing sequence,
wherein all the extension probes comprise a common nucleotide sequence for the second primer annealing sequence, and
wherein the nucleotide sequences of the first primer annealing sequence and the second primer annealing sequence are different.

6. The probe set according to any one of claims 1-5, wherein
(i) each ligation probe further comprises a molecular tag sequence, wherein the molecular tag sequence is unique for each ligation probe;
(ii) each extension probe further comprises a molecular tag sequence, wherein the molecular tag sequence is unique for each extension probe; or
(iii) each ligation probe further comprises a first molecular tag sequence and each extension probe further comprises a second molecular tag sequence, wherein the first molecular tag sequence is unique for each ligation probe, wherein the second molecular tag sequence is unique for each extension probe, and wherein the first molecular tag sequence and the second molecular tag sequence are different from each other.

7. The probe set of claim 6, wherein each molecular tag sequence is between 10 and 25 nucleotides in length.

8. A method for sequencing a mitochondrial genomic DNA comprising:
contacting a sample comprising a denatured mitochondrial genomic DNA with a probe set according to any one of claims 1-7 under conditions to permit the probe set to hybridize to the mitochondrial genomic DNA;
performing an enzymatic gap filling reaction to connect the ligation probe and the extension probe in each pair of probes, thereby producing a ligation product;
amplifying the ligation product; and
sequencing the amplified product.

9. The method of claim 8, wherein the amplifying step is achieved using a first primer that anneals to the first primer annealing sequence and a second primer that anneals to the complementary strand of the second primer annealing sequence.

10. The method of claim 8 or claim 9, wherein the sequencing is performed using next-generation sequencing.

11. The method according to any one of claims 8-10, wherein the probe pairs in the probe subsets are designed such that neighboring target regions in the heavy strand defined by the probe pairs in the first probe subset overlap with neighboring complementary target regions in the light strand defined by the probe pairs in the second probe subset.

12. The method of claim 11, wherein a target region in the heavy strand defined by a probe pair from the first probe subset is followed by an overlapping target region in the light strand defined by a probe pair from the second probe subset.

13. The method according to any one of claims 8-12, wherein each probe pair anneals to a target region that is between 200-600 nucleotides, 300-500 nucleotides, or 399-449 nucleotides in length.

14. The method according to any one of claims 8-13, wherein all the ligation probes comprise a common nucleotide sequence for the first primer annealing region,
wherein all the extension probes comprise a common nucleotide sequence for the second primer annealing region, and
wherein the nucleotide sequence of the first primer annealing region and the nucleotide sequence of the second primer annealing region are different.

15. The method according to any one of claims 8-14, wherein
(i) each ligation probe further comprises a molecular tag sequence, wherein the molecular tag sequence is unique for each ligation probe;
(ii) each extension probe further comprises a molecular tag region, wherein the molecular tag sequence is unique for each extension probe; or
(iii) each ligation probe further comprises a first molecular tag sequence and each extension probe further comprises a second molecular tag sequence,
wherein the first molecular tag sequence is unique for each ligation probe,
wherein the second molecular tag sequence is unique for each extension probe, and
wherein the first molecular tag sequence and the second molecular tag sequence are different from each other.

16. The method of claim 15, wherein each molecular tag sequence is between 10 and 25 nucleotides in length.

17. The method of claim 15, further comprising:
removing from sequencing reads sequences of the primer annealing regions, thereby producing trimmed reads;
aligning the trimmed reads based on the molecular tag regions, wherein aligned reads with identical molecular tag regions represent PCR duplicates from one probe pair and aligned reads with different molecular tag regions represent an overlapping region from different probe pairs; and
determining whether a mutation exists in the aligned trimmed reads; and
when a mutation is detected, classifying the mutation as a true variant when the mutation is found in all members of aligned reads with identical molecular tag regions, and classifying the mutation as an error when the mutation is not found in all members of aligned reads with identical molecular tag regions.

18. The method according to any one of claims 8-17, wherein the sample is from a subject having or suspected of having a mitochondrial disease selected from the group consisting of MELAS (Mitochondrial encephalopathy, lactic acidosis, and stroke-like episodes Syndrome), NARP (Neuropathy, ataxia, and retinitis pigmentosa), Leigh's Syndrome, MERRF (myoclonic epilepsy with ragged red fibers) Syndrome, Leber's hereditary optic neuropathy (LHON), Kern-Sayre Syndrome, Mitochondrial neurogastrointestinal encephalopathy syndrome (MNGIE), and Aplers Disease.

19. The method according to any one of claims 8-18, wherein the sample is from a Huntington's Disease patient.

20. A method for designing a probe set for sequencing a mitochondrial genomic DNA comprising:
designing a probe set comprising a first probe subset comprising a plurality of probe pairs and a second probe subset comprising a plurality of probe pairs,
wherein each probe pair within each probe subset comprises a ligation probe and an extension probe,
wherein each probe pair in the first probe subset comprises probes that anneal to the heavy strand of a mitochondrial genomic DNA and each probe pair in the second probe subset comprises probes that anneal to the light strand of a mitochondrial genomic DNA,
wherein each probe pair defines a target region of the mitochondrial genomic DNA that is not identical to any other target region defined by any other probe pair,
wherein the target regions defined by the first probe subset and target regions defined by the second probe subset in combination cover the entirety of the mitochondrial genomic DNA,
wherein each ligation probe comprises a first primer annealing region and a 5′-phosphorylated ligation arm that is substantially complementary to a first end of the target region on the mitochondrial genomic DNA defined by the probe pair,
wherein each extension probe comprises an extension arm that is substantially complementary to a second end of the target region on the mitochondrial genomic DNA defined by the probe pair, a molecular tag region, and a second primer annealing region,
and wherein the ligation arm does not anneal to an identical or overlapping sequence on the mitochondrial genomic DNA with the extension arm.

21. The method of claim 20, wherein the probe pairs in the probe subsets are designed such that the target regions in the heavy strand defined by the probe pairs in the first probe subset overlap with complementary target regions in the light strand defined by the probe pairs in the second probe set.

22. The method of claim 21, wherein a target region in the heavy strand defined by a probe pair from the first probe subset is followed by an overlapping target region in the light strand defined by a probe pair from the second probe subset.

23. The method according to any one of claims 20-22, wherein each probe pair anneals to a target region that is between 200-600 nucleotides, 300-500 nucleotides, or 399-449 nucleotides in length.

24. The method according to any one of claims 20-23, wherein all the ligation probes comprise a common nucleotide sequence for the first primer annealing region,
wherein all the extension probes comprise a common nucleotide sequence for the second primer annealing region, and
wherein the nucleotide sequence of the first primer annealing region and the nucleotide sequence of the second primer annealing region are different.

25. The method of claim 20, wherein
(i) each ligation probe further comprises a molecular tag sequence, wherein the molecular tag sequence is unique for each ligation probe;
(ii) each extension probe further comprises a molecular tag region, wherein the molecular tag sequence is unique for each extension probe; or
(iii) each ligation probe further comprises a first molecular tag sequence and each extension probe further comprises a molecular tag sequence, wherein the first molecular tag sequence is unique for each ligation probe, wherein the second molecular tag sequence is unique for each extension probe, and wherein the first molecular tag sequence and the second molecular tag sequence are different from each other.

26. The method of claim 25, wherein each molecular tag sequence is between 10 and 25 nucleotides in length.

27. A method of determining the mitochondrial mutation load in a subject comprising:
contacting a sample comprising a denatured mitochondrial genomic DNA with a probe set wherein the probe set comprises:
a first probe subset comprising a plurality of probe pairs and a second probe subset comprising a plurality of probe pairs,
wherein each probe pair within each probe subset comprises a ligation probe and an extension probe,
wherein each probe pair in the first probe subset comprises probes that anneal to the heavy strand of a mitochondrial genomic DNA and each probe pair in the second probe subset comprises probes that anneal to the light strand of a mitochondrial genomic DNA,
wherein each probe pair defines a target region of the mitochondrial genomic DNA that is not identical to any other target region defined by any other probe pair,
wherein the target regions defined by the first probe subset and the target regions defined by the second probe subset in combination cover the entirety of the mitochondrial genomic DNA,
wherein each ligation probe comprises a first primer annealing sequence and a 5′-phosphorylated ligation arm that is substantially complementary to a first end of the target region on the mitochondrial genomic DNA defined by the probe pair,
wherein each extension probe comprises an extension arm that is substantially complementary to a second end of the target region on the mitochondrial genomic DNA defined by the probe pair, and a second primer annealing sequence,
and wherein the ligation arm does not anneal to an identical or overlapping sequence on the mitochondrial genomic DNA with the extension arm;
performing an enzymatic gap filling reaction to connect the ligation probe and the extension probe in each pair of probes, thereby producing a ligation product;
amplifying the ligation product;
sequencing the amplified product;
removing from sequencing reads sequences of the primer annealing regions, thereby producing trimmed reads;
aligning the trimmed reads based on the molecular tag regions, wherein aligned reads with identical molecular tag regions represent PCR duplicates from one probe pair and aligned reads with different molecular tag regions represent an overlapping region from different probe pairs;
determining whether a mutation exists in the aligned trimmed reads, wherein when a mutation is detected, classifying the mutation as a true variant when the mutation is found in all members of aligned reads with identical molecular tag regions, and classifying the mutation as an error when the mutation is not found in all members of aligned reads with identical molecular tag regions; and
thereby determining the mitochondrial mutation load in a subject.

28. The method of claim 27, wherein the sequencing is performed using next-generation sequencing.

29. The method of any one of claims 27-28, wherein the probe pairs in the probe subsets are designed such that neighboring target regions in the heavy strand defined by the probe pairs in the first probe subset overlap with neighboring complementary target regions in the light strand defined by the probe pairs in the second probe subset.

30. The method of claim 29, wherein a target region in the heavy strand defined by a probe pair from the first probe subset is followed by an overlapping target region in the light strand defined by a probe pair from the second probe subset.

31. The method of any one of claims 27-30, wherein each probe pair anneals to a target region that is between 200-600 nucleotides, 300-500 nucleotides, or 399-449 nucleotides in length.

32. The method of any one of claims 27-31, wherein all the ligation probes comprise a common nucleotide sequence for the first primer annealing region,
wherein all the extension probes comprise a common nucleotide sequence for the second primer annealing region, and
wherein the nucleotide sequence of the first primer annealing region and the nucleotide sequence of the second primer annealing region are different.

33. The method of any one of claims 27-32, wherein
(i) each ligation probe further comprises a molecular tag sequence, wherein the molecular tag sequence is unique for each ligation probe;
(ii) each extension probe further comprises a molecular tag region, wherein the molecular tag sequence is unique for each extension probe; or
(iii) each ligation probe further comprises a first molecular tag sequence and each extension probe further comprises a second molecular tag sequence,
wherein the first molecular tag sequence is unique for each ligation probe,
wherein the second molecular tag sequence is unique for each ligation probe, and
wherein the first molecular tag sequence and the second molecular tag sequence are different from each other.

34. The method of claim 33, wherein each molecular tag sequence is between 10 and 25 nucleotides in length.

35. The method of any one of claims 27-34, wherein the subject is a mammal having or suspected of having a mitochondrial disease.

36. The method of claim 35, wherein the mammal is a human.

37. The method of claim 36, wherein the mitochondrial disease is selected from the group consisting of MELAS (Mitochondrial encephalopathy, lactic acidosis, and stroke-like episodes Syndrome), NARP (Neuropathy, ataxia, and retinitis pigmentosa), Leigh's Syndrome, MERRF (myoclonic epilepsy with ragged red fibers) Syndrome, Leber's hereditary optic neuropathy (LHON), Kern-Sayre Syndrome, Mitochondrial neurogastrointestinal encephalopathy syndrome (MNGIE), Aplers Disease, Huntington's Disease, Alzheimer Disease and cancer.

38. A method for determining the relative mitochondrial genomic DNA (mtDNA) content in a sample comprising:
denaturing the mtDNA and the nuclear DNA(nDNA) in the sample;
capturing a target region of the denatured mtDNA in the sample using a probe set according to any one of claims 1-7;
capturing a target region of the denatured nDNA using at least one nDNA-targeting probe pair, wherein each nDNA-targeting probe pair comprises an nDNA-targeting ligation probe and an nDNA-targeting extension probe;
determining the amount of mtDNA and the amount of nDNA; and
determining the ratio of the amount of mtDNA versus the amount of nDNA.

39. The method of claim 38,
wherein each nDNA-targeting ligation probe comprises a first primer annealing sequence and a 5′-phosphorylated ligation arm that is substantially complementary to a sequence at a first end of a target region on the nDNA defined by the probe pair;
each nDNA-targeting extension probe comprises a second primer annealing sequence and an extension arm that is substantially complementary to a sequence at a second end of the target region on the nDNA defined by the probe pair.

40. The method of claim 38 or claim 39, further comprising amplifying the captured target region of the denatured mtDNA and the captured target region of the denatured nDNA.

41. The method of any one of claims 38-41, wherein the capturing comprises performing an enzymatic gap filling reaction.

42. The method of any one of claims 38-42, wherein determining the amount of mtDNA and the amount of nDNA is achieved by next generation sequencing or by quantitative Polymerase Chain Reaction (PCR).

43. A method of determining heteroplasmy in a subject comprising:
contacting a sample comprising a denatured mitochondrial genomic DNA with a probe set of any one of claims 1-7;
performing an enzymatic gap filling reaction to connect the ligation probe and the extension probe in each pair of probes, thereby producing a ligation product;
amplifying the ligation product;
sequencing the amplified product;
removing from sequencing reads sequences of the primer annealing regions, thereby producing trimmed reads;
aligning the trimmed reads based on the molecular tag regions, wherein aligned reads with identical molecular tag regions represent PCR duplicates from one probe pair and aligned reads with different molecular tag regions represent an overlapping region from different probe pairs;
determining whether heteroplasmy exists in the aligned trimmed reads, wherein when a mutation is detected, classifying the mutation as a heteroplasmy variant when the mutation is found in an overlapping region from different probe pairs; and
thereby determining the heteroplasmy in a subject.

44. The method of claim 43, wherein the sequencing is performed using next-generation sequencing.

45. The method according to any one of claims 43-44, wherein the probe pairs in the probe subsets are designed such that neighboring target regions in the heavy strand defined by the probe pairs in the first probe subset overlap with neighboring complementary target regions in the light strand defined by the probe pairs in the second probe subset.

46. The method of claim 45, wherein a target region in the heavy strand defined by a probe pair from the first probe subset is followed by an overlapping target region in the light strand defined by a probe pair from the second probe subset.

47. The method according to any one of claims 43-46, further comprising determining the degree of heteroplasmy in the subject.
